(19) United States
(12) Patent Application Publication
(10) Pub. No.: US 2014/0154703 A1
(43) Pub. Date: Jun. 5, 2014

(54) CIRCULATING TUMOR CELL CAPTURE ON A MICROFLUIDIC CHIP INCORPORATING BOTH AFFINITY AND SIZE

(71) Applicants: Alison Skelley, Medford, MA (US); Denis Smirnov, Media, PA (US); Yi Dong, Belmont, MA (US); Keith D. Merdek, Natick, MA (US); Kam Sprott, Needham, MA (US); Walter Carney, North Andover, MA (US); Chunsheng Jiang, Reading, MA (US); Richard Huang, Chestnutt Hill, MA (US); Ioana Lupascu, Waltham, MA (US)

(72) Inventors: Alison Skelley, Medford, MA (US); Denis Smirnov, Media, PA (US); Yi Dong, Belmont, MA (US); Keith D. Merdek, Natick, MA (US); Kam Sprott, Needham, MA (US); Walter Carney, North Andover, MA (US); Chunsheng Jiang, Reading, MA (US); Richard Huang, Chestnutt Hill, MA (US); Ioana Lupascu, Waltham, MA (US)

(21) Appl. No.: 13/978,123

(22) PCT Filed: Jan. 6, 2012
(86) PCT No.: PCT/US12/20555

§ 371 Date: Feb. 20, 2014

Related U.S. Application Data
(60) Provisional application No. 61/430,509, filed on Jan. 6, 2011.
(60) Provisional application No. 61/430,930, filed on Jan. 7, 2011.
(60) Provisional application No. 61/430,891, filed on Jan. 7, 2011.
(60) Provisional application No. 61/430,897, filed on Jan. 7, 2011.

(51) Int. Cl. G01N 33/574

(52) U.S. Cl. CPC G01N 33/574
USPC 435/7.23; 435/287.2

(57) ABSTRACT

The invention encompasses methods and devices for diagnosing, theranosing, or prognosing a condition in a patient by enriching a sample in rare cells or other particles. The devices can be a microfluidic device comprising an array of obstacles and one or more binding moieties. The devices and methods can allow for enrichment of cells based on size and affinity, recovery of cells or other particles in locations on the microfluidic device, release of cells or other particles from the microfluidic device, flow of sample through the microfluidic device, and retention of rare cells or other particles from a sample obtained from a patient having a condition.

### CROSS-REFERENCE TO RELATED APPLICATIONS

This application claims the benefit of U.S. Provisional Application No. 61/430,897, filed Jan. 7, 2011, U.S. Provisional Application No. 61/430,891, filed Jan. 7, 2011, U.S. Provisional Application No. 61/430,930, filed Jan. 7, 2011, and U.S. Provisional Application No. 61/430,509, filed Jan. 6, 2011, which are hereby incorporated by reference.

### TECHNICAL FIELD

The invention relates to the fields of medical diagnostics and microfluidics.

### BACKGROUND

Cancer is a disease marked by the uncontrolled proliferation of abnormal cells. In normal tissue, cells divide and organize within the tissue in response to signals from surrounding cells. Cancer cells do not respond in the same way to these signals, causing them to proliferate and, in many organs, form a tumor. As the growth of a tumor continues, genetic alterations can accumulate, manifesting as a more aggressive growth phenotype of the cancer cells. If left untreated, metastasis, the spread of cancer cells to distant areas of the body by way of the lymph system or bloodstream, can ensue. Metastasis results in the formation of secondary tumors at multiple sites, damaging healthy tissue. Most cancer death is caused by such secondary tumors.

Despite decades of advances in cancer diagnosis and therapy, many cancers continue to go undetected until late in their development. As one example, most early-stage lung cancers are asymptomatic and are not detected in time for curative treatment, resulting in an overall five-year survival rate for patients with lung cancer of less than 15%. However, in those instances in which lung cancer is detected and treated at an early stage, the, prognosis is much more favorable. Therefore, there exists a need to develop new methods for detecting cancer at earlier stages in the development of the disease.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

In one aspect of the invention, a microfluidic device comprises an input, an output, and an array of obstacles disposed there-between and further comprising support pillars. Each of the support pillars can have a diameter of at least 100 microns and a center-to-center spacing of at least 300 microns. Each of the support pillars can have a diameter of at least 45 microns or at least 60 microns and a center-to-center spacing of at least 150 microns or at least 200 microns. Each of the support pillars can have a diameter of at least 60 microns and can be spaced less than 1000 microns away from the input. The support pillars can have a different pattern than the obstacles in the array. Each of the support pillars can have a diameter larger than the largest obstacle in the array or can have a diameter of at least 100 microns. The support pillars can be patterned in a square array. The support pillars can be spaced at least 30 microns from one another or at a distance of at least about 50% larger than any distance between said obstacles in said array. The support pillars can be less than 200 microns from the input.

In another aspect, the array of obstacles can comprise a first gap and a second gap. The second gap can be smaller than the first gap and can be situated in a repeating pattern in the array. The second gap can be distributed uniformly across the array.

In one aspect of the invention, a microfluidic device comprises a sample input, a sample output, and an array of obstacles there-between, wherein the array can have a plurality of regions. The plurality of regions can comprise a first region comprising a first gap and a second gap between a plurality of obstacles in said first region. The first gap and the second gap can be different. The plurality of regions can comprise a second region having a uniform distribution of obstacles with a single gap there-between. The second region downstream of the first region can comprise obstacles, wherein each obstacle can have a diameter smaller than the diameter of each obstacle in the first region. The second region can be downstream of the first region. The plurality of regions can further comprise one or more additional regions downstream of the second region. The one or more additional regions can have a uniform distribution of obstacles with a single gap there-between, wherein said single gap can be progressively smaller from the second region to each downstream array from the additional regions. Each of the one or more additional regions downstream of the second region can comprise obstacles with a diameter, wherein the obstacle diameter can be progressively smaller from the second region to each downstream array from the additional regions. The second gaps can be distributed in a symmetrical pattern, uniform pattern, repeating pattern, or a non-uniform pattern.

In one aspect of the invention, a microfluidic device can comprise an input, an output, and an array of obstacles disposed there-between, the array having a plurality of regions, a first region comprising a first gap and a second gap between a plurality of obstacles in the first region, wherein the first gap and the second gap can be different, and a second region comprising a first gap and a second gap between a plurality of obstacles in the second region, wherein the first gap and the second gap can be different, and wherein the first gap in the first and second regions can be the same, and wherein the second gap in the second region can be smaller than the second gap in the first region. The second region can be downstream of the first region. The second region downstream of the first region can comprise obstacles with a diameter, wherein each obstacle has a diameter smaller than the diameter of each obstacle in the first region. The microfluidic device can comprise one or more additional regions downstream of the second region, wherein each of the one or more additional regions can comprise a first gap and a second gap between a plurality of obstacles, wherein the first gap and the second gap can be different, wherein the first gap in each of the plurality of regions can be the same, and wherein the second gap can be progressively smaller from the second region to each downstream array from the additional regions. The obstacles can be arranged in a non-random pattern, repeating pattern, or uniform pattern.

In one aspect of the invention, a microfluidic device comprises an input, an output, and an array of obstacles disposed there-between, wherein at least a subset of the obstacles can be arranged in clusters, wherein each cluster can comprise at least three obstacles, wherein distances between adjacent obstacles in a cluster can be smaller than distances between the cluster and its adjacent clusters. Substantially all or all of the obstacles can be in clusters.

In a further aspect, the obstacles of any of the microfluidic devices contemplated can be coated by one or more binding moieties. The binding moieties can be affinity tagged ligands. The array of any of the microfluidic devices contemplated can comprise obstacles of various sizes. The array of any of the microfluidic devices contemplated can comprise at least 100, 200, or 300 clusters adjacent to one another. The largest distance between obstacles within a cluster can be at least three fold smaller than the smallest distance between a first cluster and a second cluster adjacent to the first cluster. The clusters of any of the microfluidic devices contemplated can comprise a longer dimension in a first direction along a flow direction than a second direction normal to the flow direction. The clusters of any of the microfluidic devices contemplated can be positioned such that a first cluster is centered upstream of a second cluster and wherein the center of the second cluster can be off-set from the center of the first cluster. The first cluster can centered upstream of a second cluster, wherein the center of the second cluster can be off-set from the center of the first cluster by an angle between about 0° to 90° or less than about 45° from a horizontal line a flow direction. The distance between obstacles in a cluster of any of the microfluidic devices contemplated can comprise less than 40, 30, 20, or 15 microns.

In another aspect, the array of any of the microfluidic devices contemplated can comprise a plurality of regions in series or in parallel, wherein clusters in each region can have a different characteristic. The characteristic can be selected from the group consisting of a different spacing between one or more obstacles within a cluster, a different spacing between clusters, angle of attachment, a different angle between clusters, a different angle between obstacles within the same cluster, or a combination thereof. Any of the devices or arrays described herein can further comprise a transition region between a first region and a second region wherein the transition region can comprise obstacles of different sizes. The array can comprise more than 4 or 5 regions.

The input of any of the microfluidic devices contemplated can be fluidly coupled to one or more additional arrays. The clusters can be arranged in a non-uniform, non-random, or a repeating pattern. The clusters can consist of three obstacles having a first and a second angle of attack each less than 45°, between about 10°-90°, between about 20°-40°, or between about 30°-40°.

In another aspect, a microfluidic device can comprise a sample input, a sample output, and an array of obstacles there-between having a first gap between a subset of said obstacles and a second gap between a second subset of said obstacles, wherein the first gap can be larger than said second gap and wherein the second gap can be distributed across the array in a non-uniform, non-random pattern. The second gaps can be distributed in a symmetrical or repeating pattern. The second gaps can be distributed such that the centers of the second gaps form virtual lines that traverse the flow direction.

In one aspect, a microfluidic flow-through device can comprise an input, an output, and an array of obstacles, wherein the array can be configured to capture at least 80% of capture entity, for example, EpCAM, expressing cells spiked into a blood sample with a volume between about 1.5 mL to about 20 mL that does not contain capture entity, for example, EpCAM, expressing cells upon flowing of the sample through the device at a rate between about 0.25 mL/hr to about 12.5 mL/hr. In one aspect, a microfluidic flow-through device can comprise an input, an output, and an array of obstacles, wherein the array can be configured to capture at least 80% of CTCs spiked into a non-CTC containing blood sample with a volume between about 1.5 mL to about 20 mL upon flowing of the sample through the device at a rate of between about 0.25 mL/hr to about 12.5 mL/hr. In one embodiment, more than 50% of captured cells can be captured in the upstream half of the array of any of the microfluidic devices contemplated. In one embodiment, more than 10% of captured cells can be captured based on size and not affinity using any of the microfluidic devices contemplated.

In one aspect of the invention, a method for enriching CTC's can comprise flowing a sample comprising CTC's through any of the microfluidic devices described herein.

In a further aspect, a method for monitoring for cancer recurrence can comprise enumerating or characterizing CTC's enriched from a plurality of samples derived from a patient at different points in time and enumerating or characterizing CTC's from the patient, and using the data to determine likelihood of cancer recurrence in the patience with at least 80% confidence level.

In one aspect of the invention, a method for monitoring treatment efficacy in a patient receiving cancer treatment can comprise the steps of enumerating or characterizing CTC's enriched from a sample from a patient derived before treatment and at least one sample derived after treatment, and using this data to determine whether a treatment can be efficacious with at least 80% confidence level.

In one aspect of the invention, a method for screening for cancer in a patient can comprise the steps of enumerating or characterizing CTC's enriched from a sample from said patient, and using this data to determine whether the patient has cancer or should seek further tests to confirm the cancer, wherein the screen has a sensitivity of at least 80%.

In a further aspect of the invention, the methods described can further comprise the steps of performing molecular analysis on CTC's captured or classifying CTC's captured, and using this information to determine the likelihood of cancer recurrence in the patient, determine whether a treatment is efficacious with at least 80% confidence level, or whether the patient has cancer or should seek further tests to confirm the cancer, or any combination thereof.

The molecular analysis can comprise sequencing, SNP detection, gene expression analysis, cDNA analysis, mRNA analysis, protein expression analysis, modified protein analysis, post-translationally modified protein analysis, mutated protein analysis, protein modification analysis, miRNA profiling, monitoring enzymatic activity, (for example from cell lysates), chromogenic in situ hybridization (CISH) analysis, or fluorescence in situ hybridization (FISH) analysis. In some embodiments, classifying can comprise identifying a sub-population of CTC's, wherein the sub-population can be characterized by the results of molecular analyses performed or the ability to be captured by a binding moiety specific for a marker in Table 1.

The methods described herein can further comprise comparing cells captured within each of one or more of the regions from the microfluidic flow-through devices described, for example, comparing the number of cells captured or the results of molecular analyses performed.

The methods described herein can further comprise a sample that can be at least 10 mL and can be processed in less than 20 hrs. In some aspects, the sample can between at least 7.5 mL to about 25 mL and can be processed in less than 20 hrs.

A surface of any of the microfluidic devices comprising an array of obstacles can be coated with one or more binding moieties. The binding moieties can comprise affinity tagged ligands. The binding moieties can comprise antibodies. The antibodies can be functionalized with a carbohydrate, for example, dextran or dextran derivatives. In one aspect, any of the microfluidic devices described herein can comprise an array of obstacles coated with antibodies wherein a surface of said device is functionalized with dextran or dextran derivatives. In one aspect, any of the microfluidic devices described herein can comprise an array of obstacles coated with antibodies, wherein a surface of said device has a contact angle of less than 15° over at least 10 hours. The affinity tagged ligands or binding moieties can enable capture of epithelial cells, non-epithelial cells, non-epithelial tumor cells, cells undergoing epithelial to mesenchymal transition, cancer stem cells, mesenchymal cells, or cellular fragments, proteins, nucleic acids particles or microparticles thereof, or any combination thereof.

Any of the described microfluidic devices can comprise a surface functionalized with two or more different polymers, wherein the first polymer can be a carbohydrate and the second polymer can be a polyethylene-glycol (PEG). A single PEG linker length can be used or two or three or more different PEG linker lengths can be used. The carbohydrate can be dextran. The carbohydrate can have a molecular weight of 10K-70K. The dextran can be at a concentration from 0.01% to 5% or from 0.05% to 2% (w/w) of the surface. The PEG can have a molecular weight of 1,000-100,000K. The PEG can have a molecular weight of 1,000-20,000K. The PEG and the carbohydrate can be at a molar ratio of 1:10 to 10:1 respectively. The surface can further comprise a binding moiety. The binding moiety can comprise avidin, an avidin derivative, NeutrAvidin, StreptAvidin, CaptAvidin, other biotin binding proteins, biotin, biotin derivatives, or other avidin binding proteins. The binding moiety can be covalently or noncovalently bonded to the carbohydrate. The binding moiety can be bonded to the carbohydrate via a linker. Linkers can comprise biotin or biotin derivatives. Linkers can comprise functional groups, for example, imide or alcohol. A linker can comprise biotin-PEG-NHS. A linker can comprise nucleic acids, amino acids, biotin-PEG-Maleimide, biotin-PEG-COOH, or biotin-PEG-SH. In one aspect, a microfluidic device can comprise an array of obstacles coated with avidin or an avidin derivative. A microfluidic device can further comprise a binding moiety of an antibody. A microfluidic device can further comprise a DNA linker.

Any of the microfluidic devices can comprise a plastic surface coupled to one or more antibodies, wherein the antibodies can be on average more than a PEG3 length from the plastic surface. The surface of any of the devices described herein can be a plastic or a cyclic olefin co-polymer (COC) or a cyclic olefin polymer (COP). A method for manufacturing the device of any of the devices described herein, can comprise manufacturing a device using a cyclic olefin co-polymer (COC) material or a cyclic olefin polymer (COP) material, wherein the COC or COP material can be molded using a blank.

A microfluidic device can be capable of capturing at least 60% of CTC's spiked into a normal blood sample, wherein the device is functionalized with a volume of an antibody solution, for example a 20 μg/mL antibody solution. The volume of the antibody solution can be between about 100 μL to about 1 mL, for example, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, or 1 mL. The concentration of the antibody solution can be between about 1 μg/mL to about 100 μg/mL, for example, 1 μg/mL, 2 μg/mL, 3 μg/mL, 4 μg/mL, 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, or 100 μg/mL.

A method for capture and release of cells or cell fragments of interest can comprise flowing a sample comprising cells or cell fragments of interest on a surface coated with carbohydrate and binding moiety that selectively bind a cell surface marker specifically present on the cells or cell fragments of interest, and using either an enzyme that selectively cleaves the carbohydrate or a biotin derivative that competitively releases biotin conjugates, or both, to thereby release the cells or cell fragments of interest from the surface. In some embodiments, an enzyme that selectively cleaves the carbohydrate can comprise dextranase, a glycosyltransferase, a glycoside hydrolase, a transglycosidase, a phosphorylase, or a lyase. In some embodiments, biotin, or a biotin derivative that competitively releases biotin or desthiobiotin conjugates can comprise biotin, desthiobiotin, or other biotin conjugates. A method for capture and release of cells or cell fragments of interest can comprise flowing a sample comprising cells or cell fragments of interest on a surface coated with a DNA linker and binding moiety that selectively binds a cell surface marker specifically present on the cells or cell fragments of interest, and using either an enzyme that selectively cleaves, for example a restriction enzyme, or nonspecifically cleaves, for example DNAse, a nucleic acid sequence within the nucleic acid sequence of the DNA linker to thereby release the cells or cell fragments of interest from the surface. A method for capture and release of cells or cell fragments of interest can comprise flowing a sample comprising cells or cell fragments of interest on a surface coated with an antibody, peptide or protein linker and binding moiety that can selectively bind a cell surface marker specifically present on the cells or cell fragments of interest, and using either a protein or peptide that competitively releases the cell, or cell fragment from an antibody or other binging moiety, or an enzyme, for example a protease such as trypsin, chymotrypsin, or elastase, that cleaves the peptide or protein linker to thereby release the cells or cell fragments of interest from the surface.

A hydrophilic linker can extend in aqueous environments and can provide maximal flexibility/solubility and activity to immobilized antibodies. Both PEG and dextran based cross-linkers can be used. In addition to high hydrophilicity as with PEG, dextran has the unique property in that it can be dissolved by dextranase under mild conditions that cause little to no damage to cells, proteins, DNAs, and RNAs. This property can be used to release capture rare species, such as CTCs, and other cancer biomarkers from the chip for advanced study.

Another option is a hydrophilic, photo-cleavable cross-linker or just a photo-cleavable cross-linker. Both can be used for photo induced release of species from blood.

### BRIEF DESCRIPTION OF THE DRAWINGS

FIG. 1—Depicted is a T7.2 microfluidic device with an array of obstacles in an exemplary arrangement.

FIG. 2—Depicted is a T7.3 microfluidic device with an array of obstacles in an exemplary arrangement.

FIG. 3—Depicted is a C5.2 microfluidic device with a plurality of regions with an array of obstacles in an exemplary arrangement. Transition zones between the end zone and plenum and between arrays are depicted.

FIG. 4—Depicted is a C5.3 microfluidic device with a plurality of regions with an array of obstacles in an exemplary arrangement.

FIG. 5—Depicted is a CS1.1 microfluidic device with an array of obstacles in clusters of three obstacles in an exemplary arrangement.

FIG. 6—Depicted is a C5.4 microfluidic device with an array of obstacles in clusters of three obstacles in an exemplary arrangement.

FIG. 7—Depicted is a C5.4 microfluidic device with an array of obstacles in an exemplary arrangement with a plenum comprising support pillars. Various pillar diameters and distances between support pillars are depicted.

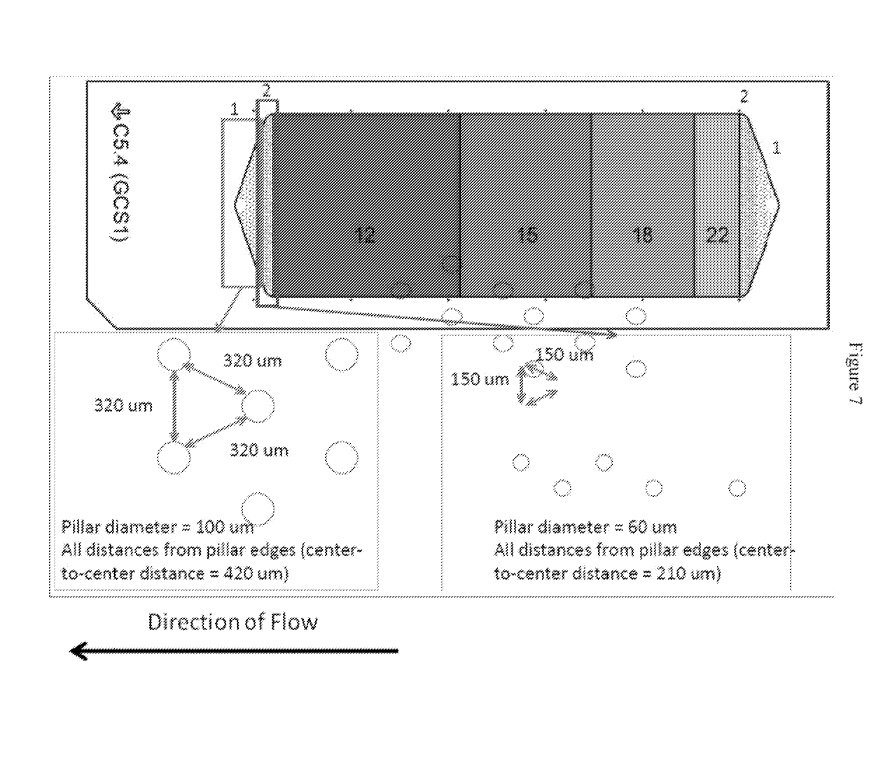

FIG. 8—Depicted is a zoomed-in view of a blood sample flowing through an array of obstacles in a microfluidic device arranged in clusters of two (top) or three (bottom) obstacles with pinch points there between.

FIG. 9—Depicted is a zoomed-in view of a blood sample flowing through an array of obstacles in a microfluidic device arranged in clusters of three (top) or four (bottom) obstacles with pinch points there between. The length from one cluster of obstacles in one column to a cluster of obstacles in an adjacent column is represented by A. The length from one cluster of obstacles in one column to an adjacent cluster of obstacles in the same column is represented by B. The width from one cluster of obstacles in one column to a cluster of obstacles in an adjacent column is represented by C. The diameter of an obstacle within a cluster of obstacles is represented by D.

FIG. 10—Depicted is a zoomed-in view of a blood sample flowing through arrays of obstacles in two regions of a microfluidic device arranged in clusters of three obstacles with larger pinch points between obstacles in the first region than the pinch points between obstacles in the second, downstream region

FIG. 11—Depicted is a microfluidic device in a parallel chamber design each chamber with a plurality of regions with four different pinch point sizes with an array of obstacles in an exemplary arrangement.

FIG. 12—Depicted is computer simulation of various flow paths of a blood sample through a microfluidic device with an arrangement of obstacles in clusters of three (top) or four (bottom) obstacles using posts of various diameters.

FIG. 13—FIG. 5—Depicted is a zoomed-in view of various cell migration paths in a blood sample flowing through an array of obstacles in a microfluidic device

FIG. 14—Depicted is a plot showing the force on cells captured within a microfluidic device as a function of the angular position of the cell or particle on the obstacle relative to the angle of the flow. The force on the cell can be greatest when the cell is on the side of the obstacle and smallest when the cell is directly in front of or behind the obstacle relative to the angle of the flow.

FIG. 15—Depicted is a plot showing the maximum shear stress on cells within a microfluidic device as a function of the gap size and tables showing the maximum shear stress for cells of various hydrodynamic sizes in various microfluidic devices.

FIG. 16—Depicted is a graph of the percentage of total capture attributable to affinity dominated capture, affinity and size mixed capture, and size dominated capture as a function of EpCAM (top graphs) and IgG (bottom) chip type using various sample volumes and flow rates.

FIG. 17—Depicted are two capture plots showing the spatial localization of cells captured by C5.4-anti-EpCAM and C5.4-anti-IgG microfluidic devices.

FIG. 18—Depicted is a graph of affinity capture as a percentage of total capture in each region of a microfluidic device with various exemplary obstacle arrangements.

FIG. 19—Depicted is a graph of the percentage of total capture using various flow rates as a function of chip type.

FIG. 20—Depicted are graphs of the percentage of capture attributable to affinity dominated capture, affinity and size mixed capture, and size dominated capture as a function of chip type using various flow rates.

FIG. 21—Depicted is a graph of the percentage of cell capture in a blood samples of various volumes as a function of anti-EpCAM and anti-IgG chip types using various flow rates.

FIG. 22—Depicted is a graph of the average percentage of total capture from a plurality of blood samples using various flow rates and sample volumes on C5.3 or C5.4 chips.

FIG. 23—Depicted are capture plots showing the spatial localization of cells captured by C5.4-anti-EpCAM coated microfluidic devices using various incubation times, sample volumes, and flow rates.

FIG. 24—Depicted are capture plots showing the spatial localization and average capture percentage (recovery percentage) of cells (H1650, PC3, and MDA-MB-231) with high, moderate, and low EpCAM expression that were spiked into a blood sample captured by C5.3-anti-EpCAM microfluidic devices.

FIG. 25—Depicted are capture plots showing the spatial localization and average capture percentage (recovery percentage) of cells (H1650, PC3, and MDA-MB-231) with high, moderate, and low EpCAM expression, spiked into a blood sample captured by C5.4-anti-EpCAM microfluidic devices.

FIG. 26—Depicted is the previously utilized array layout at the edge of the channel which can result in some obstacles close to the edge of the channel, which can result in a soft tool that can tear. Also depicted, is the new array layout design comprising arrays wherein all gaps less than 12 microns from the edge are removed and arranged at the edge of the channel as shown.

FIG. 27—Depicted are Kaplan-Meier plots of overall survival over time as a function of the sub-classification of CTCs detected in the patient.

FIG. 28—Depicted are a general scheme for capture of CTCs and other particles using microfluidic devices of the current disclosure (top) and characterization strategies for downstream analysis of captured cells and particles in the microfluidic devices (bottom).

FIG. 29—Depicted is a plot of the percent total capture of Hs578t cells spiked into a blood sample using 2 markers of the epithelial to mesenchymal transition (EMT) coated on a surface of a microfluidic device. This demonstrates that other binding moieties can be uses to capture cells with low or no EpCAM expression.

FIGS. 30A and 30B—Depicted are microfluidic devices with a two (A, top) or four (B, bottom) parallel chamber design each chamber with a plurality of regions with multiple characteristics, each with an array of obstacles in various arrangements for capture of cells, particles, or any combination thereof, from the same sample. Each chamber is shown as containing a different capture moiety and being stained with different detection moiety.

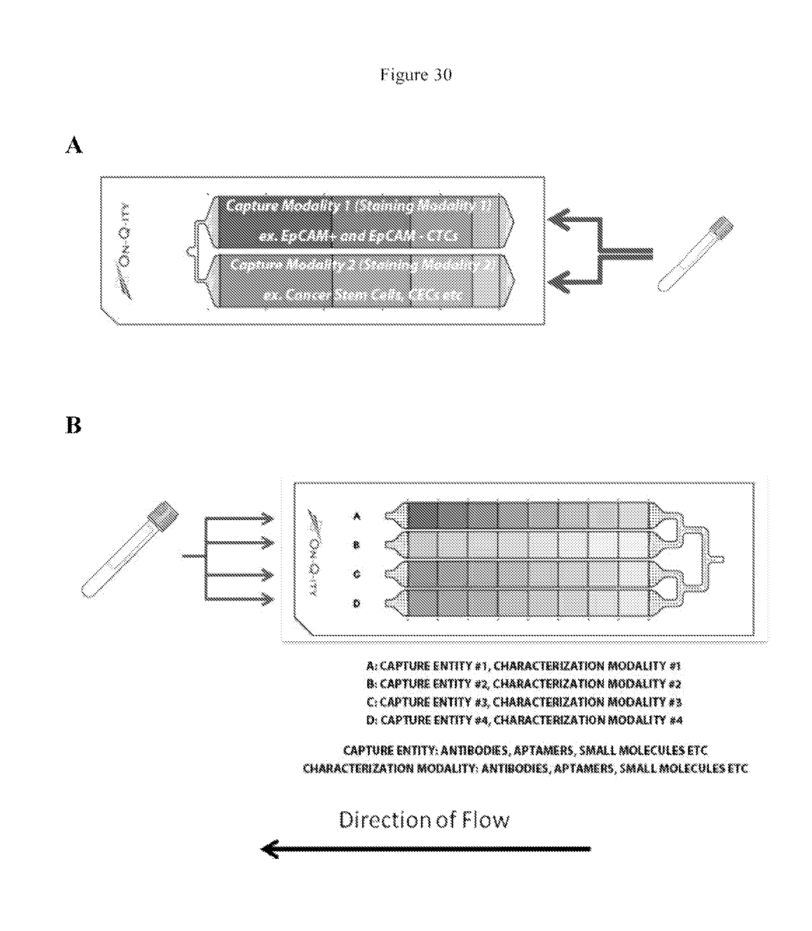

FIGS. 31A and 31B—Depicted are examples of work flow for capture of CTCs and other particles using microfluidic devices of the current disclosure and characterization strategies for downstream analysis of captured cells and particles in the microfluidic devices (A, top) and for biomarker discovery (B, bottom).

FIGS. 32A and 32B—Depicted is an example of the steps for the growth of cells within a microfluidic device following capture of the cells (A, top) and an example of the steps for the growth of cells in culture after capture within a microfluidic device following release of the captured cells (B, bottom).

FIG. 33—Depicted are examples various surface chemistries, binding moieties, and linkers that can be coated onto one or more surfaces of any of the microfluidic devices contemplated.

FIG. 34—Depicted are three examples of surface chemistry methods used in microfluidic devices of the disclosure to improve affinity capture.

FIG. 35—Depicted is a plot comparing the percent cell capture and performance using new surface chemistry methods used for affinity capture using microfluidic devices coated with different concentrations of EpCAM antibodies.

FIG. 36—are capture plots comparing the spatial localization and average capture percentage (recovery percentage) of cells in a blood sample captured by anti-IgG and anti-EpCAM functionalized microfluidic devices using either a direct covalent link or a biotin-PEG-NHS cross linker.

FIG. 37—Depicted is a heatmap of relative mRNA expression of four genes in various cells lines captured by microfluidic devices.

FIG. 38—Depicted are examples of downstream analysis methods that can be used to further characterize captured cells or particles.

FIG. 39—Depicted are computer simulations of various flow paths of a blood sample through four different regions of a C5.4 microfluidic device with an arrangement of obstacles in clusters of three obstacles.

FIG. 40—Depicted is a heat map of zoomed-in view of a blood sample flowing through an array of obstacles in a microfluidic device showing flow speed and the shear stress distribution.

FIG. 41—Depicted is table with various parameters of an exemplary C5.4 microfluidic device.

FIG. 42—Depicted is a schematic of MPS chemistry

FIGS. 43A and 43B—Depicted is a plot evaluating total cell capture percentage and chip performance using two different chip designs with H1650 and H29 cell lines and the capture percentage on EpCAM Antibody and IgG coated chips (A, top) and capture percent difference (B, bottom) between EpCAM Antibody and IgG coated chips.

FIGS. 44A and 44B—Depicted is a plot comparing the effect of using dextran of different molecular weights and PEG as a linker on reducing WBC counts (A, top) and a capture plot showing the spatial localization of cells captured by C5 devices with dextran or dextran and PEG functionalization (B, bottom).

FIG. 45—Depicted is a plot of the effect of added BSA on the amount of antibodies immobilized on a chip surface as quantified by alkaline phosphatase/PNPP assay.

FIGS. 46A and 46B—Depicted are plots of the cell capture rate of IgG control chips and EpCAM chips at different antibody concentrations (A, top) and the difference in capture rates of the two chips when NeutrAvidin is covalently linked to the surface (B, bottom).

FIGS. 47A and 47B—Depicted are plots of the cell capture rate of IgG control chips and EpCAM chips at different antibody concentrations (A, top) and the difference in capture rates of the two chips when NeutrAvidin is linked to the surface via a hydrophilic cross-linker (B, bottom).

FIG. 48—Depicted are capture plots showing the spatial localization of cells captured by C5 IgG control chips and EpCAM chips when NeutrAvidin is covalently linked to the surface and when NeutrAvidin is linked to the surface via a hydrophilic cross-linker.

FIG. 49—Depicted are various alternative obstacle arrangements in arrays of microfluidic devices (top) and tables with various parameters of a microfluidic device with four chambers for multiparameter processing of a sample (bottom).

FIG. 50—Depicted are capture plots showing the spatial localization of cells and total cell percentage captured by IgG control chips and EpCAM chips with the indicated obstacle array arrangements.

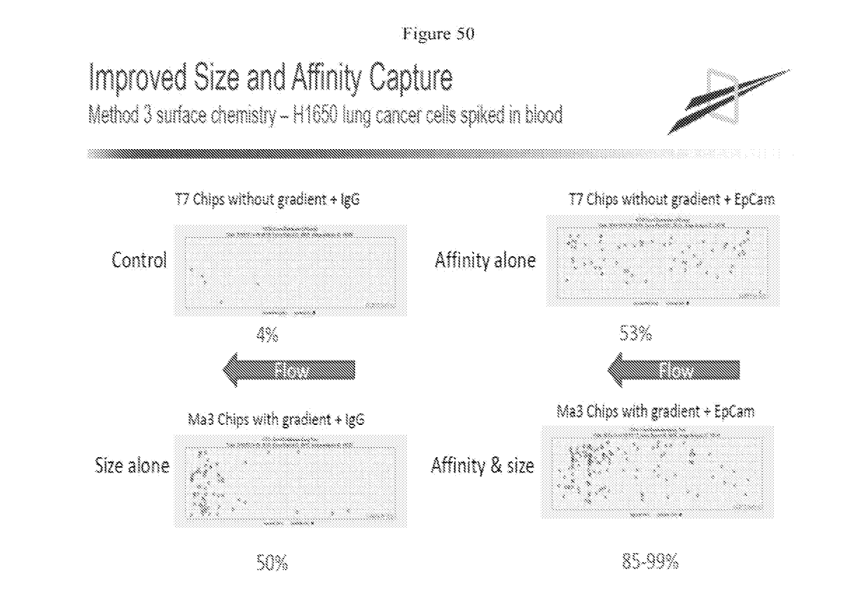

FIG. 51—Depicted are the total capture percentage using the C5.1 and C5.2 designs compared to the original C5 design at 25 μL/min.

FIG. 52—Depicted are capture plots showing the spatial localization of cells and total cell percentage captured by IgG control chips and EpCAM chips spiked in either PBS or blood samples.

FIG. 53—Depicted are the total capture percentage using the C5.1 and C5.2 designs using various flow rates, surface chemistries, and blood sample types.

FIG. 54—Depicted are capture plots showing the spatial localization of cells using the C5.1 and C5.2 designs using various flow rates and surface chemistries.

FIG. 55—Depicted is a graph of number of cells recovered vs. the number of cells spiked into a sample process on the C5.2 chip design demonstrating linear capture of cells spiked into the sample ranging from 0-750 spiked cells.

FIG. 56—Depicted are graphs of the total capture percentage from samples spiked with a known number of CTCs were processed on the C5.2, C5.3, and C5.4 designed chips functionalized with either IgG or EpCAM antibodies at a flow rate of either 4 μL/min, or 8 μL/min.

FIG. 57—Depicted are capture plots showing the spatial localization of cells from samples spiked with a known number of CTCs and processed on the C5.2, C5.3, and C5.4 chip designs functionalized with either IgG or EpCAM antibodies at a flow rate of either 4 μL/min, or 8 μL/min.

FIG. 58—Depicted are graphs of the total cell capture percentage in various zones processed using C5.3 (top) and C5.4 (bottom) chip designs, using 7 hr and 4 hr antibody incubation times and various flow rates.

FIG. 59—Depicted is a graph of the total cell capture percentage from 3.75 mL blood samples processed on the C5.2, C5.3, and C5.4 chip designs functionalized with EpCAM using at flow rates of 4 μL/min, 25 μL/min, and 75 μL/min under the same antibody incubation times using three different blood samples.

FIG. 60—Depicted are capture plots showing the spatial localization of cells from 3.75 mL blood samples spiked with a known number of CTCs and processed on the C5.2, C5.3, and C5.4 chip designs functionalized with EpCAM antibodies at a flow rate of either 4 μL/min.

FIG. 61—Depicted are capture plots showing the spatial localization of cells from 3.75 mL blood samples spiked with a known number of CTCs and processed on the C5.2, C5.3, and C5.4 chip designs functionalized with EpCAM antibodies at a flow rate of either 25 μL/min.

FIG. 62—Depicted are capture plots showing the spatial localization of cells from 3.75 mL blood samples spiked with a known number of CTCs and processed on the C5.2, C5.3, and C5.4 chip designs functionalized with EpCAM antibodies at a flow rate of either 75

FIG. 63—Depicted is a graph of the total number of captured leukocytes from the blood (non-specific capture) from three different 3.75 mL blood samples processed on the C5.2, C5.3, and C5.4 chip designs functionalized with EpCAM using at flow rates of 4 μL/min, 25 μL/min, and 75 μL/min under the same antibody incubation times.

FIG. 64—Depicted is a graph of the total cell capture percentage of 3 different cell lines using 4 different blood samples with a volume of 3.75 mL at a flow rate of either 4 μl/min or 8 μL/min using the same number of spiked cells under the same processing conditions processed on the C5.2 and C5.4 chip designs functionalized with EpCAM.

FIG. 65—Depicted are capture plots showing the spatial localization of 3 different cell lines into blood samples with a volume of 3.75 mL at a flow rate of 4 μl/min or 7.5 mL at a flow rate of 8 μl/min using the same number of spiked cells under the same processing conditions processed on the C5.2 chip designs functionalized with EpCAM.

FIG. 66—Depicted are capture plots showing the spatial localization of 3 different cell lines into blood samples with a volume of 3.75 mL at a flow rate of 4 μl/min or 7.5 mL at a flow rate of 8 μl/min using the same number of spiked cells under the same processing conditions processed on the C5.4 chip designs functionalized with EpCAM.

FIG. 67—Depicted is a table summarizing some of the key results from experiments comparing various parameters using the C5.2, C5.3, and C5.4 chip designs.

### DEFINITIONS

By “antibodies” is meant any immunoglobulin molecules and immunologically active portions of immunoglobulin molecules, i.e., molecules that contain antigen-binding sites that specifically bind an antigen. A molecule that specifically binds to a polypeptide of the disclosure is a molecule that binds to that polypeptide or a fragment thereof, but does not substantially bind other molecules in a sample, for example, a biological sample, which naturally contains the polypeptide. Examples of immunologically active portions of immunoglobulin molecules include F(ab) and F(ab′)2 fragments which can be generated by treating the antibody with an enzyme such as pepsin and other techniques known in the art. The disclosure provides polyclonal and monoclonal antibodies that bind to a polypeptide of the disclosure. The term “monoclonal antibody” or “monoclonal antibody composition”, as used herein, refers to a population of antibody molecules that contain only one species of an antigen binding site capable of immunoreacting with a particular epitope of a polypeptide of the disclosure.

By “about” in the context of length, size, area, or other measurements is meant equal to within 10%, 5%, 4%, 3%, 2%, or even 1%.

By “biological sample” is meant any sample of biological origin or containing, or potentially containing, biological particles. Preferred biological samples are cellular samples.

By “blood component” is meant any component of whole blood, including host red blood cells, white blood cells, platelets, or epithelial cells, in particular, CTCs. Blood components also include the components of plasma, for example, proteins, lipids, nucleic acids, and carbohydrates, and any other cells that can be present in blood, for example, because of current or past pregnancy, organ transplant, infection, injury, or disease.

By “cell fragment or particle” is meant any species of biological origin that is insoluble in aqueous media. Examples include particulate cell components, viruses, and complexes including proteins, lipids, membranes, nucleic acids, and carbohydrates.

By “cellular sample” is meant a sample containing cells or components thereof. Such samples include naturally occurring fluids (for example, blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal or genitourinary tract, amniotic fluid, and water samples) and fluids into which cells have been introduced (for example, culture media and liquefied tissue samples). The term also includes a lysate.

By “channel” is meant a gap through which fluid can flow. A channel can be a capillary, a conduit, or a strip of hydrophilic pattern on an otherwise hydrophobic surface wherein aqueous fluids can be confined.

By “circulating tumor cell” (CTC) is meant any rare cell comprising features of non-normal morphology, histology, and gene expression patterns that disseminate from a primary tumor organ sited location. CTCS can use the circulating blood as a conduit for migration to distal locations or secondary metastatic sites and can result in metastatic disease. CTCs may comprise epithelial cells, mesenchymal cells, cells undergoing epithelial to mesenchymal transition (EMT), cells undergoing mesenchymal to epithelial transition (MET), cancer stem cells, or other rare cell lineages. Other rare cell lineages can comprise circulating endothelial cells as well as circulating stem cells.

By “component” of cell is meant any component of a cell that can be at least partially isolated from a cell using methods known in the art, for example, lysis. Cellular components can be organelles (for example, nuclei, perinuclear compartments, nuclear membranes, mitochondria, chloroplasts, or cell membranes), polymers or molecular complexes (for example, lipids, polysaccharides, proteins (membrane, trans-membrane, or cytosolic), nucleic acids (native, therapeutic, or pathogenic), viral particles, or ribosomes), microparticles (for example, particles of various cell origin), or other molecules (for example, hormones, ions, cofactors, or drugs).

By “component” of a cellular sample is meant a subset of cells, or components thereof, contained within the sample.

By “density” in reference to an array of obstacles is meant the number of obstacles per unit of area, or alternatively the percentage of volume occupied by such obstacles. Array density can be increased either by placing obstacles closer together or by increasing the size of obstacles relative to the gaps between obstacles or a combination thereof. Array density can be decreased either by placing obstacles farther apart or by decreasing the size of obstacles relative to the gaps between obstacles.

By “enriched sample” is meant a sample containing components that can be processed to increase the relative population of components of interest relative to other components typically present in a sample. For example, samples can be enriched by increasing the relative population of cells of interest by at least 10%, 25%, 50%, 75%, 100% or by a factor of at least 1,000, 10,000, 100,000, 1,000,000, 10,000,000, or even 100,000,000.

By “gap” is meant an opening through which fluids or particles can flow. For example, a gap can be a capillary, a space between two obstacles wherein fluids can flow, or a hydrophilic pattern on an otherwise hydrophobic surface wherein aqueous fluids can be confined.

By “hydrodynamic size” is meant the effective size of a particle when interacting with a flow, obstacles, or other particles. It is used as a general term for particle volume, shape, and deformability in the flow.

By “intracellular activation” is meant activation of second messenger pathways leading to transcription factor activation, or activation of kinases or other metabolic pathways. Intracellular activation through modulation of external cell membrane antigens can also lead to changes in receptor trafficking.

By “labeling reagent” is meant a reagent that is capable of binding to an analyte, being internalized or otherwise absorbed, and being detected, for example, through shape, morphology, color, fluorescence, luminescence, phosphorescence, absorbance, magnetic properties, or radioactive emission.

By “microfluidic” is meant having at least one dimension of less than 1 mm.

By “microstructure” in reference to a surface is meant the microscopic structure of a surface that includes one or more individual features measuring less than 1 mm in at least one dimension. Exemplary microfeatures can be micro-obstacles, micro-posts, micro-grooves, micro-fins, and micro-corrugations.

By “obstacle” is meant an impediment to flow in a channel, for example, a protrusion from one surface or a post. For example, an obstacle can refer to a post outstanding on a base substrate or a hydrophobic barrier for aqueous fluids. The obstacle can be impermeable or partially permeable. For example, an obstacle can be a post made of porous material, wherein the pores allow penetration of an aqueous component but can be too small for the particles being separated to enter.

### DETAILED DESCRIPTION OF THE INVENTION

The invention features devices and methods for detecting, enriching, and analyzing circulating tumor cells (CTCs) and other particles. The invention further features methods of diagnosing a condition in a subject, for example, cancer, by analyzing a cellular sample from the subject. Devices of the invention can include arrays of obstacles that allow displacement of CTCs, other rare cells, cellular derivatives, cellular components, biological entities, or other fluid components. Some objectives of the new designs comprise improving priming of device by eliminating corners of plenum where bubbles can be frequently trapped, adding support to the tape to prevent collapse into the plenum during assembly and processing, adding embossing support to the smallest pillars, utilizing more of the capture area by combining the most effective capture zones in the current designs, exploring the relationship between capture efficiency for a range of cell types (both specific and non-specific) and the gap size, angle of pinch point relative to flow, and density of pinch points, constructing parallel capture chamber geometry to begin testing priming and operation of a multi-chamber design, maintaining high capture efficiency of C5 designs while reducing shear on cells passing through array, reducing drag forces on captured cells to encourage greater affinity capture, providing redundancy of capture in relevant size ranges, and relying upon computer simulations to optimize array geometry based on flow visualization/simulation results.

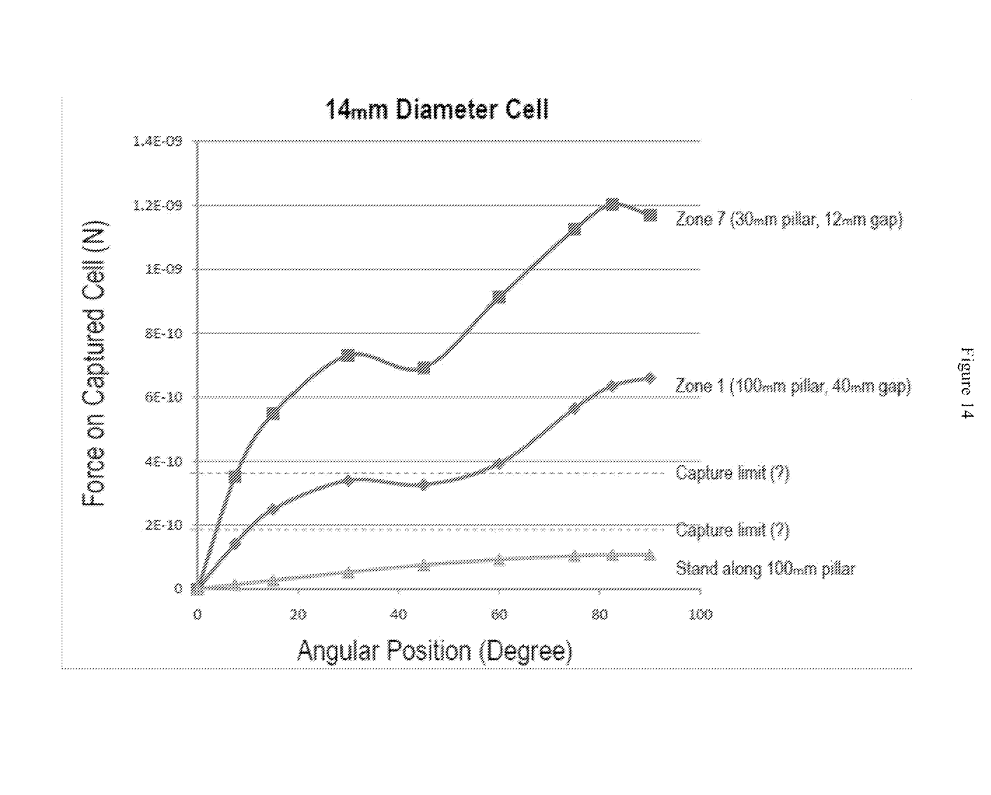

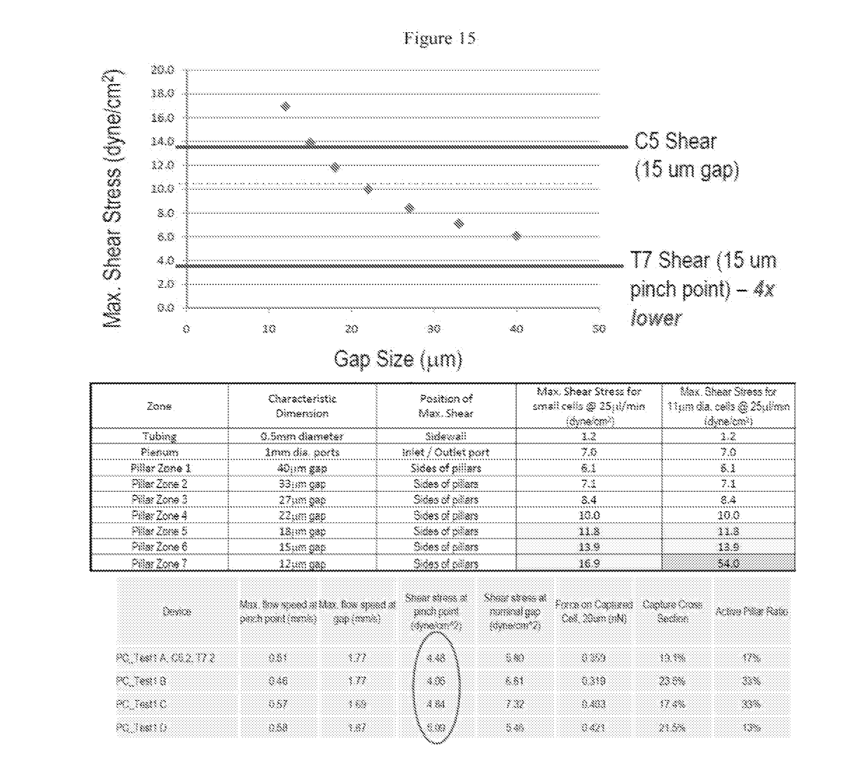

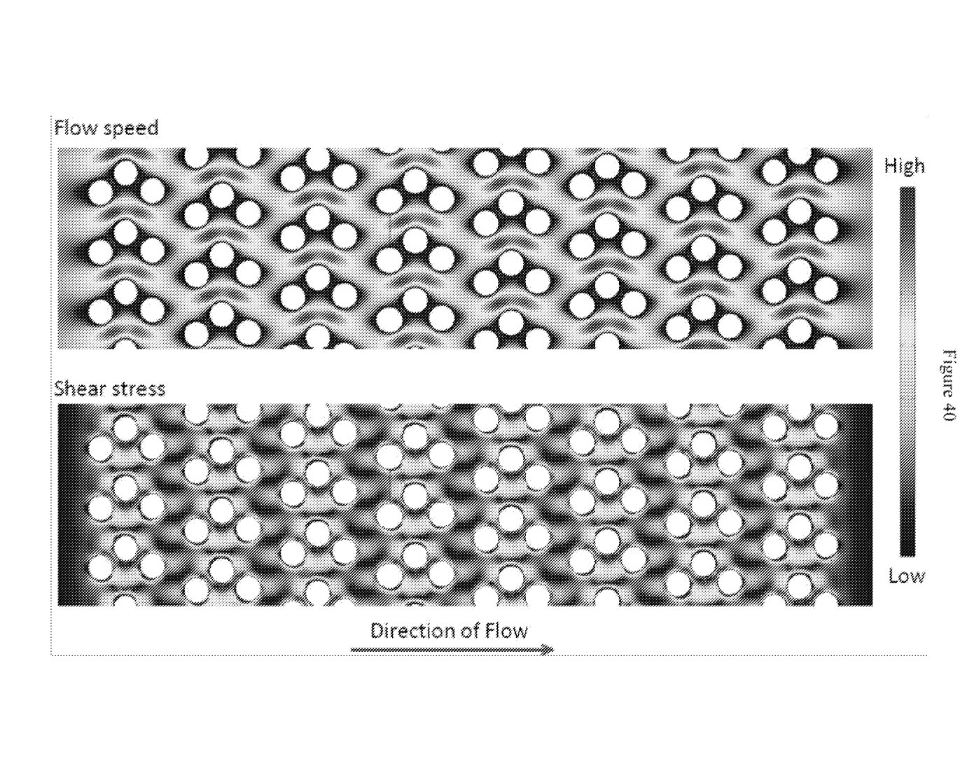

Although the previous C5 and C5.1 designs can have high capture efficiency, the location of capture can occur in the last regions of the array. Furthermore, these designs can be a challenge to manufacture through embossing, can be prone to damage, the feature sizes of the chip may not be amenable to injection molding, and can demonstrate high shear forces on cells and high forces on captured cells (FIGS. 14 and 15). Although suitable for comparison studies based on enumeration, for affinity capture and characterization, and for stable long-term manufacturing, new geometries of obstacles and gaps were designed. Some of the advantages to the new gap geometries comprise a reduction of shear and drag forces on cells (FIG. 40), a larger affinity component to the capture mechanism (easier to make clear distinction between EpCAM- and EpCAM+ cells), a base gap region that can reduce forces on cells thus enabling processing at higher flow rate, and larger base gaps and larger obstacles that results in more stable manufacturing and can be more amenable to injection molding.

Microfluidic methods can be effective means to interrogate the constituents of biological fluids for diagnostic purposes, just as they can be useful for precise measurements and assays for other analytical processes, such as drug screening, nucleic acid amplification, and enzymatic reactions. A particular microfluidics challenge for analysis of CTCs is the necessity for evaluating relatively large sample volumes to access key information about rare cells in circulation. Thus, the small dimensional features of chip design, and complex fluid dynamics can interfere with efficient, high scale capture of specific, rare cells unless its format and microfluidics can be styled to meet specific requirements. Cells emerging from a cancer can be distinguished by any of their molecular features; yet it can be a challenging problem to absolutely identify CTCs. A central dilemma is that the CTC attributes are diverse, and therefore a selection of the cell-based features has been informative.

Furthermore, although circulating cells, microparticles, cellular fragments, proteins and nucleic acids have an enormous diagnostic potential, it can be a challenge to efficiently capture these materials from biological fluids. Biological fluids, such as blood, often contain vast numbers of normal cells and materials that can be irrelevant for diagnostic purposes. Moreover, presence of such materials can render the possibility of such diagnostics difficult due to low signal to noise ratios. Thus, there is a need for efficient enrichment methods that allow for very efficient capture of useful disease-related materials, yet minimize capture of other irrelevant biological materials. The current disclosure features uniquely formatted and structured devices for processing a cellular sample.

Enumeration and characterization of one or more rare cells, such as CTCs, using the devices and methods herein can be useful in assessing cancer diagnosis, theranosis, and prognosis, including, for example, early cancer detection, early detection of treatment failure, and detection of cancer relapse. Enumeration and characterization of one or more rare cells using the devices and methods herein can also be useful in selecting and monitoring therapy in a patient.

In addition to enrichment of circulating cells, microparticles, cellular fragments, proteins and nucleic acids, characterization of captured material can be useful to obtain diagnostic information. Moreover, quantitative comparison between circulating cells, microparticles, cellular fragments, proteins, nucleic acids, or any combination thereof with various characteristics may be required in order to obtain reliable diagnostic information. This can be difficult to accomplish using limited amounts of biological samples that can be routinely obtained from patients. The methods and devices of the current disclosure can be used to address these difficulties.

Although the detection, enrichment, and analysis of rare cells such as CTCs or epithelial cells is a preferred embodiment of this application, the devices and methods of the invention can be useful for processing a wide range of other cells, fragments, analytes, and particles. The cells and particles can be cancer cells, circulating tumor cells (CTCs), epithelial cells, circulating endothelial cells (CECs), circulating stem cells (CSCs), stem cells, undifferentiated stem cells, cancer stem cells, bone marrow cells, progenitor cells, foam cells, fetal cells, mesenchymal cells, circulating epithelial cells, circulating endometrial cells, trophoblasts, immune system cells (host or graft), connective tissue cells, bacteria, fungi, pathogens (for example, bacterial or protozoa), microparticles, cellular fragments, proteins, nucleic acids (i.e., DNA or RNA), membranes, cellular organelles, liposomes, nucleosomes, exosomes, other cellular components (for example, mitochondria and nuclei), and viruses.

Circulating cells of various origins can be detected in the blood stream of patients with various diseases. For example, CTCs and CSCs can be identified in peripheral blood of cancer patients. Increased number of CECs and endothelial progenitor cells (EPCs) can be found in blood of patients with a disease, for example cancer, cardiovascular disease, systemic lupus erythematosus (SLE), diabetes and various other diseases associated with endothelial dysfunction.

A primary tumor contains a heterogeneous cell population that can be composed of tumor cells and normal tissue supporting stroma and endothelium, and inflammatory cells. All can contribute to the rapid expansion in size, vascularization capacity, genetic instability, nutrient deprivation, normoxia and hypoxia, reprogramming, necrosis, shedding, or any combination thereof. Thus, the dynamic and heterogeneous features of tumors can form a daunting array of biomolecules, particles, cells, and cell aggregates that pass into the blood. Many of the constituents released from tumors may not themselves able to form metastatic colonizing cells. Nonetheless, these agents can supply relevant signs of tumor progression, and can also be a source of biomarkers and other indicators of disease status and response. For example, microvesicles of tumor origin can be readily purified from the cancer patient bloodstream without any cellular contamination. These microvesicles can be continuously shed by the tumor (cells) into the circulation, whereas comparable microvesicle generation of non-tumor origin can be rare.

The devices and methods of the current disclosure are designed to enable surveying of subcellular constituents, biomolecules, particles, cells, and cell aggregates for example, free or complexed protein, liposomes, RNA, DNA, microparticles, or subcellular particles such as nucleosomes and exosomes. While other technologies can selectively interrogate only portions of the materials released from tumors and fail to allow the combined isolation and analysis of tumor cells and the subsequent analysis of constituents of these cells, the microfluidic devices and methods of their use as described herein can provide flexible platforms with advantages of combining CTC capture with a variety of molecular analyses.

Microparticles (MPs) in human blood can originate from platelets and can also be released from leukocytes, erythrocytes, endothelial cells and other cells. As a non-limiting example, endothelial microparticles can be small vesicles that can be released from endothelial cells and can be found circulating in the blood. The microparticle comprises a plasma membrane surrounding a small amount of cytosol. The membrane of the endothelial microparticle contains receptors and other cell surface molecules which enable the identification of the endothelial origin of the microparticle, and allow it to be distinguished from microparticles from other cells, such as platelets. They can be important mediators of cellular processes, such as cancer progression, inflammation, coagulation, and vascular homeostasis. In addition, MPs can also carry various nucleic acid species as cargo and can be detected in small amounts in the blood of normal individuals. Elevated platelet-derived MP (PDMP), endothelial cell-derived MP (EDMP), and monocyte-derived MP (MDMP) concentrations are documented in almost all thrombotic diseases occurring in both venous and arterial beds. In cancer patients, MPs allow ‘non-genetic’ intercellular transfer that provides a pathway for the cellular acquisition and dissemination of traits between cancer cells such as multidrug resistance (MDR).

Increased numbers of microparticles, for example, circulating endothelial microparticles, have been identified in individuals with certain diseases (i.e., hypertension, cardiovascular disorders, pre-eclampsia, and various forms of vasculitis). The endothelial microparticles in some disease states have been shown to have arrays of cell surface molecules indicate a state of endothelial dysfunction. Therefore, endothelial microparticles can be used as an indicator or index of the biological state of the endothelium in disease, and may play roles in the pathogenesis of certain diseases.

Any of the rare cells, particles, or any combination thereof as described herein can be obtained from a sample from a patient. A rare cell can be one that can be up to 0.5%, 1%, 5%, or 10% of all cells in the sample. A sample can be any cellular, preferably, fluidic sample, from the patient.

The bodily fluid can be blood (such as peripheral blood), serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, fecal matter, hair, tears, cyst fluid, pleural fluid, peritoneal fluid, pericardial fluid, lymph, chyme, chyle, bile, interstitial fluid, menses, pus, sebum, vaginal flow or secretion, mucosal secretion, stool water, pancreatic juice, lavage fluid from sinus cavities, bronchopulmonary aspirate, blastocyl cavity fluid, bone marrow suspension, cerebrospinal fluid, brain fluid, ascites, milk, secretions of the respiratory, intestinal, or genitourinary tract, amniotic fluid, a water sample, or umbilical cord blood. The biological sample can also be blastocyl cavity or umbilical cord blood. The biological sample can also be a tissue sample or biopsy. A typical sample is a blood sample. A fluidic sample from a patient or one that has been solubilized can be at least about 1, 2, 3, 4, 5, 6, 7, 7.5, 8, 9, 10, 20, 50, 75, 100, 200, 500, 1000 or 1500 mL or greater than 5, 7.5, 10, 50, 75, 100, 500, or 750 mL. Exemplary devices and methods of the invention are described in detail below.

### Chip Designs

The microfluidic devices of the present disclosure are uniquely designed to facilitate cell or particle capture by microfluidics and surface interactions in a complex process. The main factors for capture can include affinity association (for example, through tumor antigen recognition), cell or particle size, and cell or particle specific adhesion properties. In addition, the known properties of cells or particles that can be important to be excluded can be significant.

The design and implemented features of the microfluidic devices described herein can address both the known properties of CTCs, particles, and non-tumor blood cells. These features can include the different sizes of the circulating cancer cells, the differing levels of expression of tumor surface antigens, and the variability in adhesion properties. The development of high surface to volume ratio can be an important technical principle in the microfluidic capture. The microfluidic devices and the capture technology of the current disclosure can consist of a dual capture mechanism, affinity and size. In some aspects, the microfluidic devices and the capture technology of the current disclosure can comprise a single capture mechanism, wherein the capture mechanism can be affinity or size. Standard protein chemistry can immobilize antibodies onto a plastic surface enabling classic affinity capture. A gradient pattern of posts (C5 design) with decreasing gap distances can trap tumor cells while allowing smaller red and white blood cells to pass through. Enrichment of CTCs through both mechanisms has the potential of increasing capture efficiency, thereby providing a broader array of cancer cells for later analysis and characterization.

The microfluidic devices described previously and herein comprise a field of posts in a hollow chamber through which the microfluidic flow passes the cell and microparticle containing sample for capture. The previous designs of the devices incorporated a staggered distribution of posts in order to maximize contact between cells and surfaces. This capture surface was established by fabrication of circular columns, or microposts (100 μm diameter, 100 μm height) that are arranged in a linear pattern across the surface of the chip. These previous devices consisted of deep-etched silicon surfaces composed of an array of 78,000 posts in the device's capture zone. Plastic has since been utilized, improving both the reliability as well as providing for more versatility for surface chemistry.

The devices of the current disclosure contain post dimensions that can be modulable depending on the prototype formed. In describing the post distributions, some of the most important features can include, but are not limited to, the number of posts, the diameters of posts, the gap sizes between posts of equivalent or different sizes, the arrangement of the posts, and the zones of posts of equivalent or different sizes in the entire microfluidic post field between the inlet and outlet of the device. These attributes have been engineered into different device designs and are described herein.

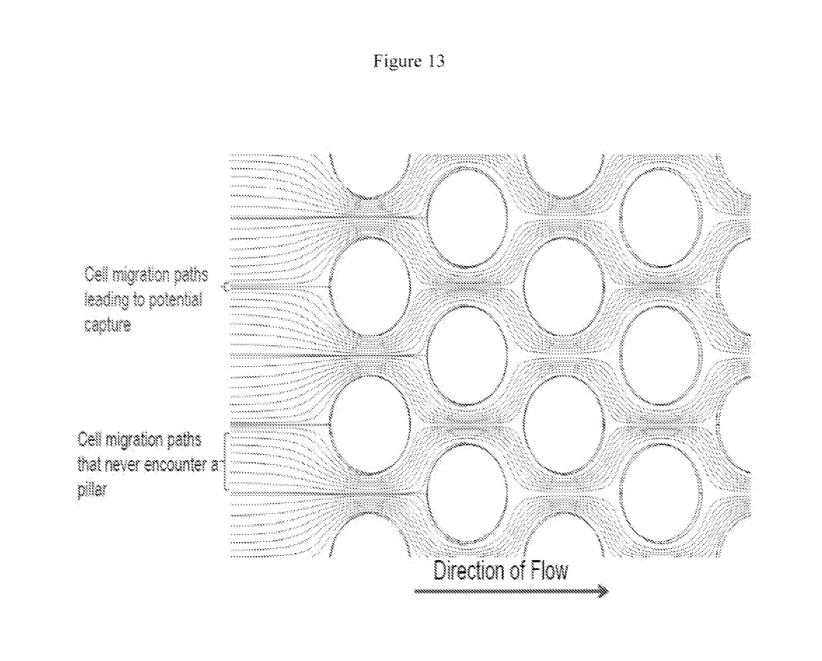

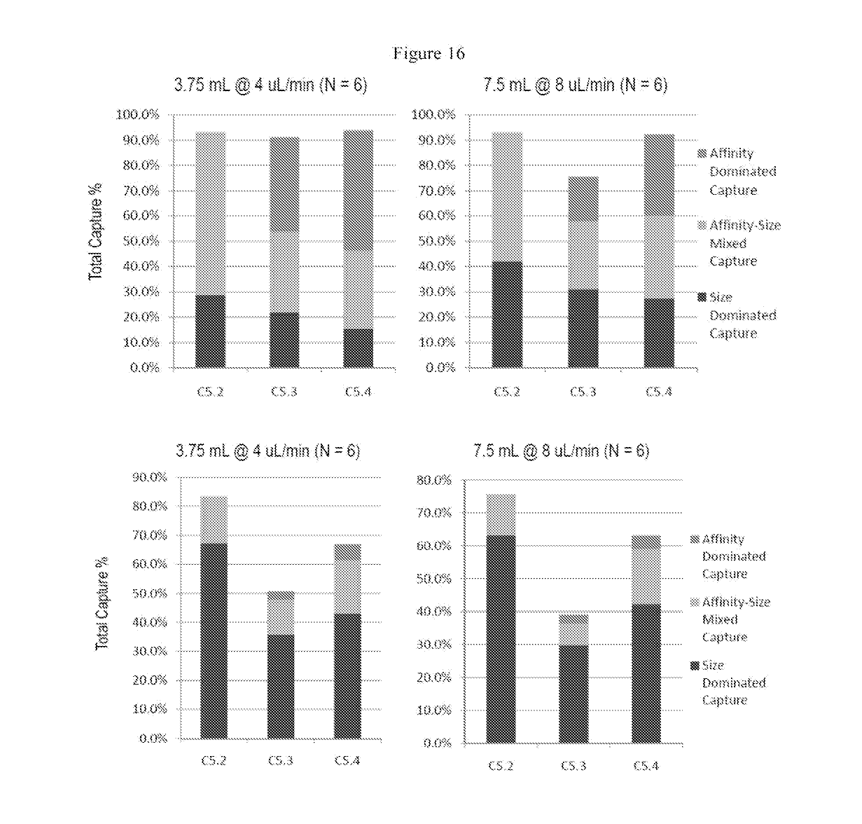

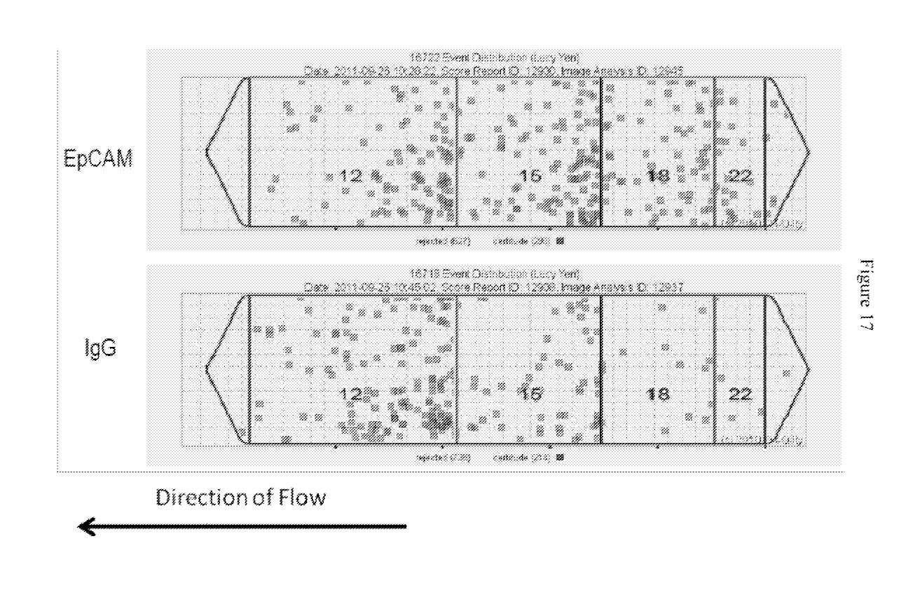

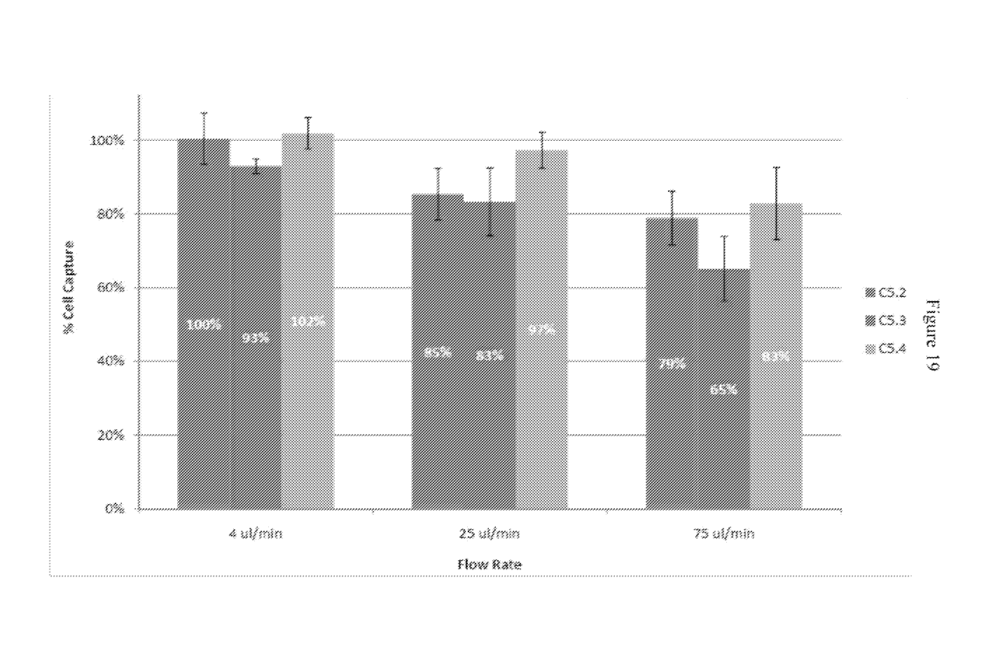

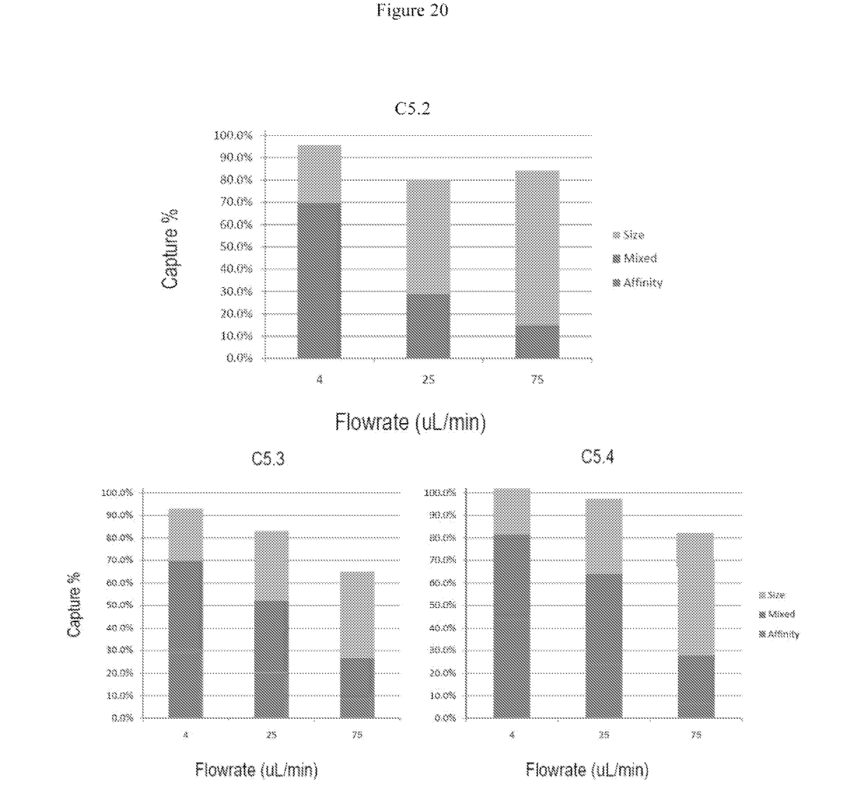

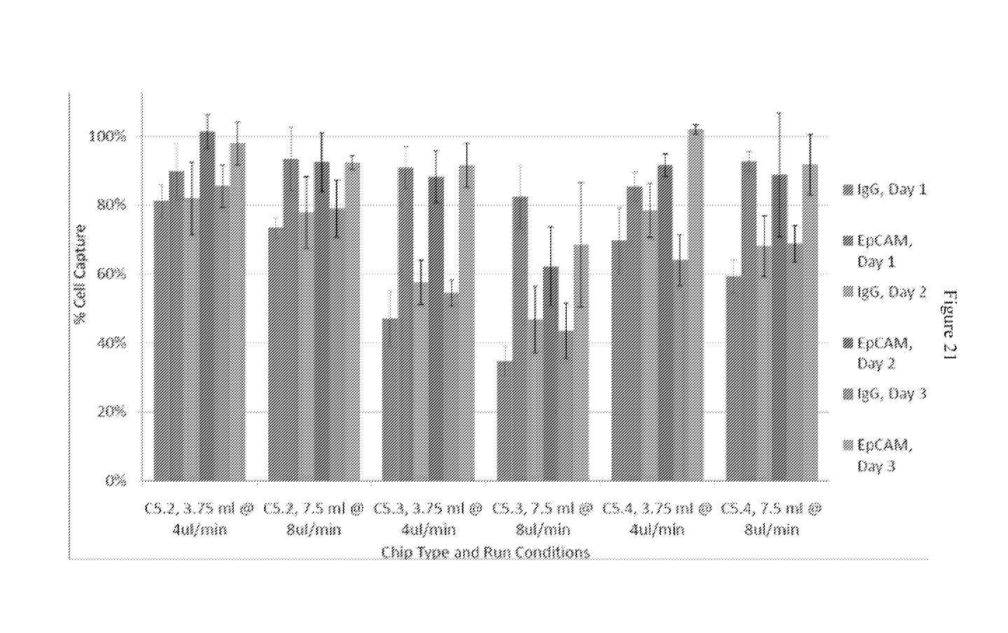

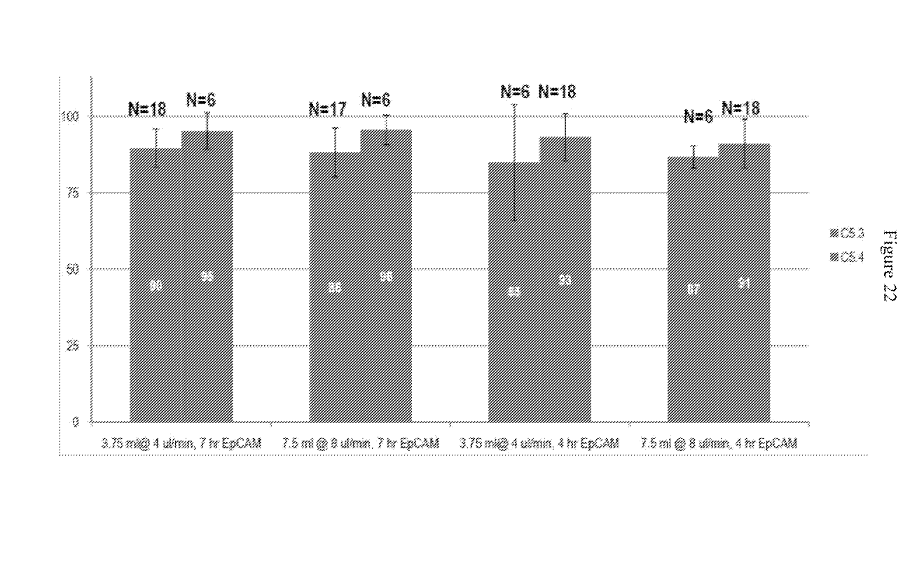

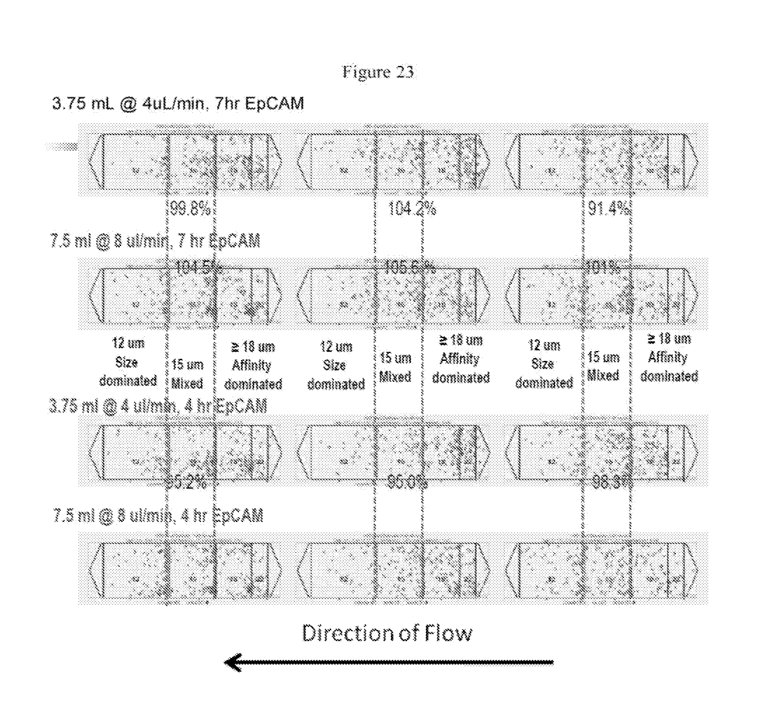

The microfluidic devices described herein offers several system strengths as the technologies can be directed towards applications in different cancers or other diseases or conditions. When flowing across a plastic chip and through a maze, arrangement, or array of posts, shear forces can be minimized (FIG. 40), to diminish any further damage to the cells, or to prevent dislodging the cells from their interaction with surface binding moieties, such as antibodies. Normal cellular components in the blood that outnumber the CTCs by billions to one, can also exert massive physical forces on the captured CTCs as they flow across the chip surface. The arrays of obstacles can be arranged to maintain high capture efficiency of previous designs and further reduce shear on cells passing through an array (FIG. 15), reduce drag forces on captured cells (FIG. 14), and increase the capture cross-section to promote greater affinity capture (FIG. 13). The arrays of obstacles can be arranged to promote uniform fluid flow across the entire channel and exposure of the cells to the obstacles for both affinity and size capture depending on the region where capture occurs. Moreover, such designs can allow for higher flow rates to be maintained without significant loss in total capture efficiency (FIG. 19, FIG. 20 and FIG. 21). Additionally, the surface of the microfluidic device can be functionalized with binding moieties for less time than previously utilized methods (FIG. 22 and FIG. 23). Furthermore, the designs can provide redundancy of cell capture in a relevant size range, which can provide information as to the means by which the cells were captured, for example, by size or affinity (FIG. 16 and FIG. 17 and FIG. 18 and FIG. 20).

Devices of the invention can be employed to produce a sample enriched in cells or particles of a desired hydrodynamic size. Applications of such enrichment include concentrating CTCs or other cells of interest, and size fractionization, for example, size filtering (selecting cells in a particular size range). Devices can also be used to enrich components of cells or particles, for example, nuclei or other constituent fragmented cellular components described herein. Desirably, the methods of the invention can retain at least about 50%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the desired particles compared to the initial mixture, while potentially enriching the desired particles by a factor of at least about 100, 1,000, 10,000, 100,000, 1,000,000, 10,000,000, or even 100,000,000 relative to one or more non-desired particles. Desirably, if a device produces any output sample in addition to the enriched sample, this additional output sample can contain less than about 50%, 25%, 20%, 19%, 18%, 17%, 16%, 15%, 14%, 13%, 12%, 11%, 10%, 9%, 8%, 7%, 6%, 5%, 4%, 3%, 2%, or 1%, or even none of the desired particles compared to the initial mixture. The enrichment can also result in a dilution of the enriched particles compared to the original sample, although the concentration of the enriched particles relative to other particles in the sample may have increased. Preferably, the dilution can be at most about 90%, for example, at most about 75%, 50%, 33%, 25%, 10%, or 1%.

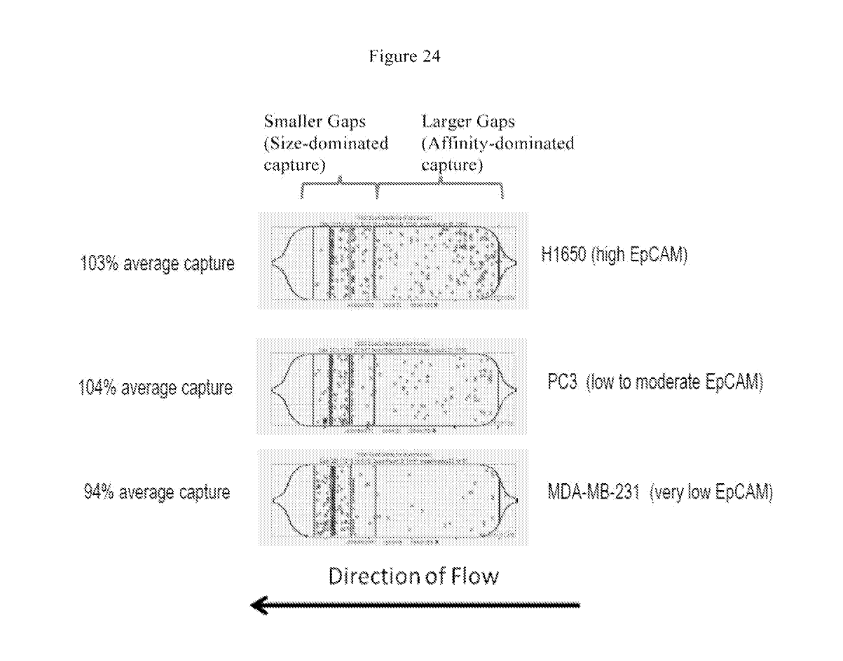

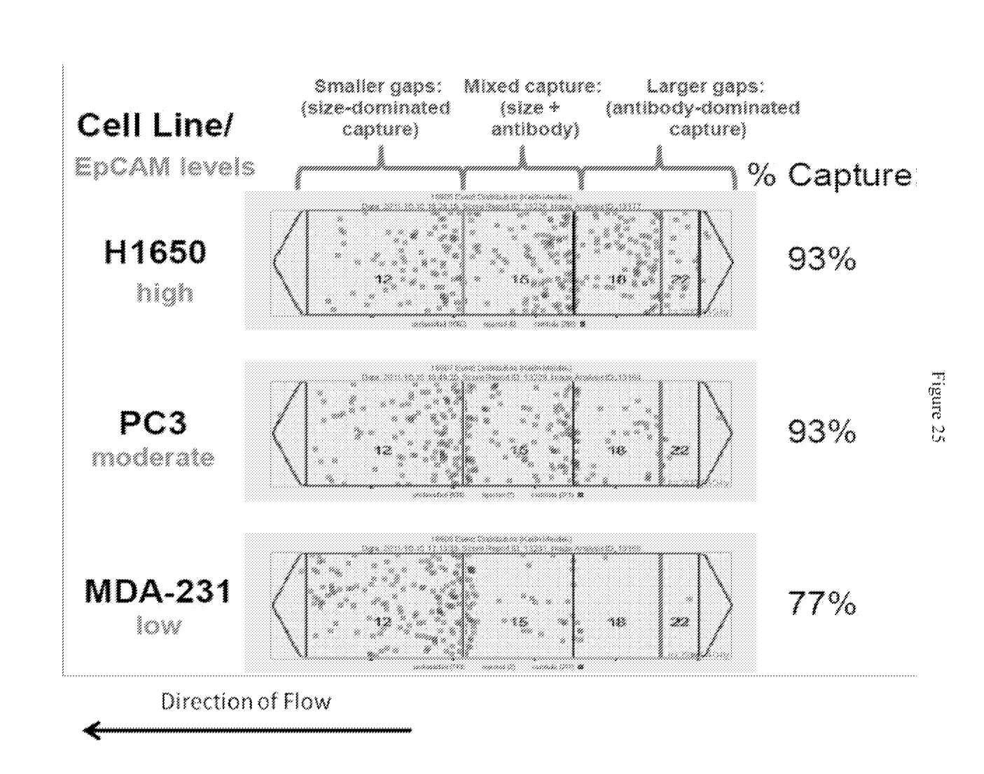

The microfluidic devices described herein can combine both affinity capture, such as through immobilized binding moieties, for example, anti-EpCAM antibodies, and size capture, for example, through a gradient system of posts with various gap sizes. This dual capture mechanism can be valuable because of the heterogeneity of tumor cells. Furthermore, the level of expression of many targeting moieties specific to the cells and particles, for example, tumor cells expression of EpCAM on their surface, captured by the methods using the devices described herein, can vary drastically. For example, some CTCs can express high levels of EpCAM while other CTCs can express low or undetectable levels of EpCAM. The designs of the current disclosure can allow for efficient total and affinity mediated capture of these cells and particles even for cells and particles with low expression of the targeting moieties (FIG. 24 and FIG. 25). These designs can not only promote affinity capture, but also allow for characterization of captured cells and particles and for more stable, long-term manufacturing.

It is known that not all CTCs express EpCAM (EpCAM-) and thus cannot be captured by affinity using EpCAM binding moieties coated on surfaces of the microfluidic devices. Both EpCAM− cells and EpCAM+ cells can be captured in the devices of the current disclosure because the devices can capture cells based on size and affinity. Therefore, affinity-based capture alone can select for the high expresser subgroup of CTCs. Furthermore, tumor cells also vary in size, and small cells can pass through filters that trap cells greater than a certain diameter. Using a dual mechanism can allow for capture of cells that either mechanism alone can miss. Capturing more cells can allow for greater and more complete characterization of these cells.

Any of the microfluidic devices described herein can comprise an input, an output, and an array of obstacles, wherein the array can be configured to capture at least about 80% of cells or particles expressing a particular targeting moiety that have been spiked into a volume of blood sample, for example 7.5 mL, that does not contain cells or particles that express the targeting moiety upon flowing the spiked sample through any of the devices of the current disclosure at a flow rate. For example, the array can be configured to capture at least about 80%, 80%, 81%, 82%, 83%, 84%, 85%, 86%, 87%, 88%, 89%, 90%, 91%, 92%, 93%, 94%, 95%, 96%, 97%, 98%, 99%, or 100% of cells or particles expressing a particular targeting moiety that have been spiked into a volume of blood sample, for example 7.5 mL, that does not contain cells or particles that express the targeting moiety, such as EpCAM, upon flowing the spiked sample through any of the devices of the current disclosure a flow rate of 0.25 mL/hr or higher. The targeting moiety can be any of the targeting moieties in Table 1 or any moiety that can specifically bind to the cells or particles desired to be captured, for example EpCAM. The flow rate of the sample through any of the microfluidic devices described herein can be at least about 0.01 ml/hr or at least about 0.25 ml/hr, for example, 0.01 mL/hr, 0.02 mL/hr, 0.03 mL/hr, 0.04 mL/hr, 0.05 mL/hr, 0.06 mL/hr, 0.07 mL/hr, 0.08 mL/hr, 0.09 mL/hr, 0.1 mL/hr, 0.15 mL/hr, 0.2 mL/hr, 0.3 mL/hr, 0.4 mL/hr, 0.5 mL/hr, 0.6 mL/hr, 0.7 mL/hr, 0.8 mL/hr, 0.9 mL/hr, 1 mL/hr, 1.1 mL/hr, 1.2 mL/hr, 1.3 mL/hr, 1.4 mL/hr, 1.5 mL/hr, 1.6 mL/hr, 1.7 mL/hr, 1.8 mL/hr, 1.9 mL/hr, 2 mL/hr, 2.1 mL/hr, 2.2 mL/hr, 2.3 mL/hr, 2.4 mL/hr, 2.5 mL/hr, 2.6 mL/hr, 2.7 mL/hr, 2.8 mL/hr, 2.9 mL/hr, 3 mL/hr, 3.1 mL/hr, 3.2 mL/hr, 3.3 mL/hr, 3.4 mL/hr, 3.5 mL/hr, 3.6 mL/hr, 3.7 mL/hr, 3.8 mL/hr, 3.9 mL/hr, 4 mL/hr, 4.1 mL/hr, 4.2 mL/hr, 4.3 mL/hr, 4.4 mL/hr, 4.5 mL/hr, 4.6 mL/hr, 4.7 mL/hr, 4.8 mL/hr, 4.9 mL/hr, 5 mL/hr, 6 mL/hr, 6.5 mL/hr, 7 mL/hr, 7.5 mL/hr, 8 mL/hr, 8.5 mL/hr, 9 mL/hr, 9.5 mL/hr, 10 mL/hr, 19.5 mL/hr, 11 mL/hr, 11.5 mL/hr, 12 mL/hr, 12.5 mL/hr, 13 mL/hr, 13.5 mL/hr, 14 mL/hr, 14.5 mL/hr, or 15 mL/hr. The microfluidic devices can be designed such that more than about 50%, for example, 55%, 60%, 65%, 70%, 75%, 80%, 85%, 90%, 95%, 96%, 97%, 98%, 99%, or 100% of the captured cells can be captured in an upstream portion, segment, or region of the array. The microfluidic devices can be designed such that more than about 10%, for example, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the captured cells can be captured based on size and not affinity. The microfluidic devices can be designed such that more than about 10%, for example, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the captured cells can be captured based on affinity and not size. Capture based on size can be determined by determining the location, portion, segment, or region within the array where the capture occurs. As a non-limiting example, cells captured within the downstream half of the array can be said to be captured by size and not affinity.

Any of the microfluidic devices described herein can comprise an input, an output, and an array of obstacles, wherein the array can be configured to capture at least about 80% of cells or particles, such as CTCs, that have been spiked into a volume of sample, for example, a 1 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, 10, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, or 40 mL volume of sample upon flowing the spiked sample through any of the devices of the current disclosure at a flow rate. For example, the array can be configured to capture at least about 80%, for example, 81%, 82%, 83%, 84%, 85%, 86%, 87%, 88%, 89%, 90%, 91%, 92%, 93%, 94%, 95%, 96%, 97%, 98%, 99%, or 100% of cells or particles that have been spiked into a volume of sample, for example, a 1 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, 10, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, or 40 mL volume of sample upon flowing the spiked sample through any of the devices of the current disclosure a flow rate of 0.25 mL/hr or higher. The targeting moiety can be any of the targeting moieties in Table 1, or, for example, EpCAM. The flow rate can be at least about 0.01 ml/hr or at least about 0.25 ml/hr, for example, 0.01 mL/hr, 0.02 mL/hr, 0.03 mL/hr, 0.04 mL/hr, 0.05 mL/hr, 0.06 mL/hr, 0.07 mL/hr, 0.08 mL/hr, 0.09 mL/hr, 0.1 mL/hr, 0.15 mL/hr, 0.2 mL/hr, 0.3 mL/hr, 0.4 mL/hr, 0.5 mL/hr, 0.6 mL/hr, 0.7 mL/hr, 0.8 mL/hr, 0.9 mL/hr, 1 mL/hr, 1.1 mL/hr, 1.2 mL/hr, 1.3 mL/hr, 1.4 mL/hr, 1.5 mL/hr, 1.6 mL/hr, 1.7 mL/hr, 1.8 mL/hr, 1.9 mL/hr, 2 mL/hr, 2.1 mL/hr, 2.2 mL/hr, 2.3 mL/hr, 2.4 mL/hr, 2.5 mL/hr, 2.6 mL/hr, 2.7 mL/hr, 2.8 mL/hr, 2.9 mL/hr, 3 mL/hr, 3.1 mL/hr, 3.2 mL/hr, 3.3 mL/hr, 3.4 mL/hr, 3.5 mL/hr, 3.6 mL/hr, 3.7 mL/hr, 3.8 mL/hr, 3.9 mL/hr, 4 mL/hr, 4.1 mL/hr, 4.2 mL/hr, 4.3 mL/hr, 4.4 mL/hr, 4:5 mL/hr, 4.6 mL/hr, 4.7 mL/hr, 4.8 mL/hr, 4.9 mL/hr, 5 mL/hr, 6 mL/hr, 6.5 mL/hr, 7 mL/hr, 7.5 mL/hr, 8 mL/hr, 8.5 mL/hr, 9 mL/hr, 9.5 mL/hr, 10 mL/hr, 10.5 mL/hr, 11 mL/hr, 11.5 mL/hr, 12 mL/hr, 12.5 mL/hr, 13 mL/hr, 13.5 mL/hr, 14 mL/hr, 14.5 mL/hr, or 15 mL/hr. The microfluidic device can be designed to such that more than about 50%, for example, 55%, 60%, 65%, 70%, 75%, 80%, 85%, 90%, 95%, or 99% of the captured cells can be captured in the upstream half of the array. The microfluidic devices can be designed such that more than about 10%, for example 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the captured cells can be captured based on size and not affinity. Capture based on size can be determined by determining the location, portion, segment, or region within the array where the capture occurs. As a non-limiting example, cells captured within the downstream half of the array can be said to be captured by size and not affinity.

Any of the microfluidic devices described herein can comprise an input, an output, and an array of obstacles capable of capturing at least about 60% of CTCs spiked into a normal blood sample, wherein the device was coated with a volume of a solution at 20 μg/mL concentration of one or more binding moieties, for example an antibody. The volume of the antibody solution can be between about 100 μL to about 1 mL or 2 mL, for example, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1 mL, 1.5 mL, or 2 mL. The concentration of the antibody solution can be between about 1 μg/mL to about 100 μg/mL, for example, 1 μg/mL, 2 μg/mL, 3 μg/mL, 4 μg/mL, 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, or 100 μg/mL. The microfluidic devices can be capable of capturing at least about 60% of CTCs spiked into a normal blood sample, for example, at least about 65%, 70%, 75%, 80%, 85%, 90%, 95%, or 99% of CTCs spiked into a normal blood sample.

TABLE 1
Markers for characterization of CTCs with mesenchymal characteristics

| | | | | | | | Median | | |
|---|---|---|---|---|---|---|---|---|---|
| | | | | | | | Expression | | Corr to |
| Affy ID | Gene Symbol | BR:HS 578T | BR:MDA-N | BR:MDA-MB-435 | CO:COLO 205 | CO:HT29 | in PCBM | Corr to VIM | EpCAM |
| 211719_x_at | FN1 | 13.39 | 8.01 | 7.41 | 4.87 | 5.34 | 4.73 | 0.58 | −0.46 |
| 201616_s_at | CALD1 | 11.08 | 7.8 | 7.43 | 4.47 | 4.2 | 4.2 | 0.61 | −0.54 |
| 209210_s_at | FERMT2 | 10.49 | 7.38 | 7.25 | 4.25 | 4.24 | 4.32 | 0.59 | −0.47 |
| 213139_at | SNAI2 | 10.09 | 9.68 | 9.67 | 4.05 | 4.28 | 3.77 | 0.56 | −0.57 |
| 201617_x_at | CALD1 | 10 | 6.61 | 6.37 | 3.22 | 3.14 | 3.8 | 0.5 | −0.48 |
| 201445_at | CNN3 | 9.84 | 8.37 | 8.17 | 4.42 | 4.22 | 4.05 | 0.42 | −0.23 |
| 201147_s_at | TIMP3 | 9.82 | 7.25 | 7.25 | 4.09 | 4.15 | 4.53 | 0.31 | −0.37 |
| 201150_s_at | TIMP3 | 9.63 | 6.49 | 6.8 | 4.4 | 4.4 | 4.74 | 0.32 | −0.35 |
| 209656_s_at | TMEM47 | 9.36 | 8.31 | 8.17 | 3.67 | 3.54 | 4.12 | 0.35 | −0.24 |
| 202620_s_at | PLOD2 | 9.34 | 6.95 | 6.7 | 6.23 | 4.35 | 3.94 | 0.35 | −0.4 |
| 202619_s_at | PLOD2 | 9.32 | 6.74 | 6.83 | 6.34 | 5.21 | 4.76 | 0.37 | −0.39 |
| 203324_s_at | CAV2 | 9.16 | 7.28 | 6.85 | 6.02 | 5.35 | 4.47 | 0.45 | −0.14 |
| 210139_s_at | PMP22 | 9.12 | 10.01 | 9.66 | 5.02 | 5.1 | 4.83 | 0.57 | −0.6 |
| 213194_at | ROBO1 | 8.82 | 7.98 | 7.25 | 4.23 | 4.22 | 4.19 | 0.43 | −0.43 |
| 219410_at | TMEM45A | 8.82 | 6.79 | 7.43 | 4.06 | 3.98 | 4.69 | 0.54 | −0.56 |
| 221881_s_at | CLIC4 | 8.77 | 6.36 | 6.43 | 5.83 | 5.79 | 4.64 | 0.53 | −0.45 |
| 201110_s_at | THBS1 | 8.72 | 6.48 | 6.7 | 3.16 | 3.45 | 4.88 | 0.41 | −0.28 |
| 207030_s_at | CSRP2 | 8.66 | 7.81 | 8.13 | 4.75 | 4.62 | 4.25 | 0.41 | −0.18 |
| 212551_at | CAP2 | 8.5 | 6.39 | 6.54 | 5.66 | 5.54 | 4.85 | 0.32 | −0.3 |
| 203789_s_at | SEMA3C | 8.35 | 6.31 | 6.18 | 7.03 | 5.66 | 4.45 | 0.21 | −0.19 |
| 211651_s_at | LAMB1 | 8.29 | 7.18 | 7.24 | 6.19 | 5.66 | 4.27 | 0.34 | −0.16 |
| 204688_at | SGCE | 8.06 | 7.72 | 7.46 | 4.28 | 4.42 | 4.77 | 0.48 | −0.21 |
| 206307_s_at | FOXD1 | 8.05 | 7.29 | 7.05 | 5.75 | 4.69 | 4.78 | 0.29 | −0.23 |
| 204654_s_at | TFAP2A | 7.99 | 7.36 | 6.83 | 5.23 | 4.89 | 4.37 | 0.1 | −0.19 |
| 201505_at | LAMB1 | 7.88 | 7.27 | 6.61 | 5.15 | 4.49 | 3.45 | 0.38 | −0.24 |
| 202133_at | WWTR1 | 7.86 | 9.08 | 9.1 | 4.72 | 3.93 | 3.72 | 0.57 | −0.4 |
| 211300_s_at | TP53 | 7.86 | 7.46 | 7.13 | 6.24 | 5.98 | 4.62 | −0.13 | 0.08 |
| 212094_at | PEG10 | 7.8 | 7.75 | 8.12 | 4.34 | 4.24 | 5 | 0.32 | −0.07 |
| 214612_x_at | MAGEA6 | 7.76 | 10.74 | 10.66 | 4.03 | 5.4 | 4.02 | 0.28 | −0.35 |
| 219631_at | LRP12 | 7.61 | 6.25 | 6.06 | 3.87 | 4.45 | 4.51 | 0.63 | −0.58 |
| 204235_s_at | GULP1 | 7.59 | 8.24 | 8.13 | 6.7 | 5.95 | 3.96 | 0.29 | −0.12 |
| 204030_s_at | SCHIP1 | 7.57 | 7.51 | 7.43 | 4.43 | 4.51 | 4.52 | 0.57 | −0.4 |
| 213010_at | PRKCDBP | 7.47 | 8.1 | 7.45 | 6.35 | 5.26 | 4.9 | 0.51 | −0.33 |
| 218656_s_at | LHFP | 7.38 | 6.38 | 6.39 | 4.61 | 4.4 | 4.31 | 0.55 | −0.52 |
| 205895_at | PTPN11 | 7.37 | 6.78 | 6.54 | 6.55 | 5.97 | 4.72 | −0.17 | 0.02 |
| 221428_s_at | TBL1XR1 | 7.31 | 7.37 | 6.8 | 7.12 | 5.94 | 4.33 | −0.08 | 0.05 |
| 203637_s_at | MID1 | 7.27 | 8.32 | 8.3 | 6.48 | 4.39 | 4.51 | 0.27 | −0.25 |
| 212230_at | PPAP2B | 7.27 | 6.76 | 6.63 | 6.01 | 5.8 | 4.67 | 0.28 | −0.17 |
| 219502_at | NEIL3 | 7.23 | 6.74 | 6.53 | 5.96 | 5.79 | 4.8 | 0.11 | −0.32 |
| 216693_x_at | HDGFRP3 | 7.22 | 6.92 | 6.83 | 4.76 | 4.81 | 4.72 | 0.56 | −0.51 |
| 217787_s_at | GALNT2 | 7.14 | 6.99 | 6.2 | 4.93 | 4.26 | 4.43 | 0.33 | −0.43 |
| 213306_at | MPDZ | 7.14 | 7.51 | 7.13 | 5.82 | 5.82 | 4.29 | 0.49 | −0.5 |
| 214321_at | NOV | 7.13 | 8.77 | 8.25 | 3.74 | 4.1 | 4.21 | 0.42 | −0.39 |
| 203626_s_at | SKP2 | 7.06 | 6.82 | 6.97 | 6.68 | 5.44 | 4.99 | −0.09 | 0.04 |
| 203705_s_at | FZD7 | 7.06 | 6.71 | 6.48 | 5.18 | 5.68 | 4.18 | 0.25 | −0.21 |
| 201141_at | GPNMB | 6.99 | 10.75 | 10.49 | 4.39 | 4.62 | 4.54 | 0.3 | −0.39 |
| 214724_at | DIXDC1 | 6.95 | 6.37 | 6.32 | 5.56 | 5.31 | 4.32 | 0.3 | −0.27 |
| 215913_s_at | GULP1 | 6.95 | 7.34 | 7.6 | 6.22 | 5.11 | 4.84 | 0.23 | −0.04 |
| 218330_s_at | NAV2 | 6.91 | 8.25 | 7.81 | 8.2 | 5.23 | 4.29 | 0.43 | −0.3 |
| 208653_s_at | CD164 | 6.91 | 6.61 | 6.58 | 7.9 | 5.78 | 4.55 | −0.24 | 0.13 |
| 212775_at | OBSL1 | 6.89 | 7.74 | 7.42 | 5.11 | 3.84 | 4.07 | 0.35 | −0.23 |
| 211814_s_at | CCNE2 | 6.87 | 6.6 | 6.58 | 6.35 | 5.57 | 4.45 | −0.08 | −0.16 |
| 204237_at | GULP1 | 6.84 | 8.48 | 7.72 | 5.97 | 5.32 | 3.58 | 0.26 | −0.1 |
| 213943_at | TWIST1 | 6.83 | 6.8 | 6.2 | 4.4 | 4.23 | 3.94 | 0.46 | −0.56 |
| 204955_at | SRPX | 6.79 | 10.62 | 10.47 | 4.2 | 4.31 | 4.06 | 0.52 | −0.55 |
| 204944_at | PTPRG | 6.74 | 6.49 | 6.04 | 4.1 | 3.84 | 4.12 | 0.44 | −0.44 |
| 212619_at | TMEM194A | 6.64 | 6.09 | 6.03 | 5.94 | 5.66 | 4.94 | −0.25 | 0.16 |
| 214720_x_at | 40796 | 6.64 | 7.07 | 6.4 | 5.96 | 5.96 | 3.98 | 0.27 | −0.13 |
| 210904_s_at | IL13RA1 | 6.61 | 7.57 | 7.38 | 6.52 | 5.81 | 4.97 | 0.21 | −0.22 |
| 215509_s_at | BUB1 | 6.54 | 6.84 | 6.31 | 6.14 | 5.96 | 4.37 | 0.07 | −0.06 |
| 205034_at | CCNE2 | 6.46 | 6.85 | 6.68 | 5.78 | 5.84 | 3.64 | −0.05 | −0.2 |
| 203874_s_at | SMARCA1 | 6.42 | 6.38 | 6.28 | 4.18 | 5.54 | 3.81 | 0.38 | −0.35 |
| 213756_s_at | HSF1 | 6.4 | 6.01 | 6.3 | 6.24 | 5.94 | 4.66 | −0.19 | 0.2 |
| 206907_at | TNFSF9 | 6.35 | 6.69 | 6.35 | 6.34 | 5.83 | 4.89 | 0.11 | 0.13 |
| 202755_s_at | GPC1 | 6.35 | 6.21 | 6.2 | 5.63 | 5.62 | 4.76 | 0.04 | 0.02 |
| 210467_x_at | MAGEA12 | 6.25 | 9.74 | 9.78 | 4.03 | 4.82 | 4.55 | 0.25 | −0.32 |
| 214880_x_at | CALD1 | 6.23 | 6.01 | 6.16 | 4.87 | 5.8 | 4.44 | 0.5 | −0.4 |
| 210138_at | RGS20 | 6.22 | 6.64 | 6.11 | 4.71 | 4.49 | 4.35 | 0.38 | −0.28 |
| 214297_at | CSPG4 | 6.21 | 6.71 | 6.68 | 4.4 | 4.26 | 4.68 | 0.34 | −0.39 |
| 215017_s_at | FNBP1L | 6.21 | 6.23 | 6.21 | 5.69 | 5.8 | 4.19 | −0.16 | 0.18 |
| 221643_s_at | RERE | 6.07 | 6.04 | 6.15 | 5.06 | 5.21 | 4.79 | −0.04 | 0 |
| 219650_at | ERCC6L | 6.03 | 6.67 | 6.42 | 5.64 | 5.96 | 4.32 | 0.31 | −0.33 |
| 208938_at | PRCC | 6 | 6.67 | 6.14 | 6.04 | 5.75 | 4.87 | −0.24 | 0.17 |

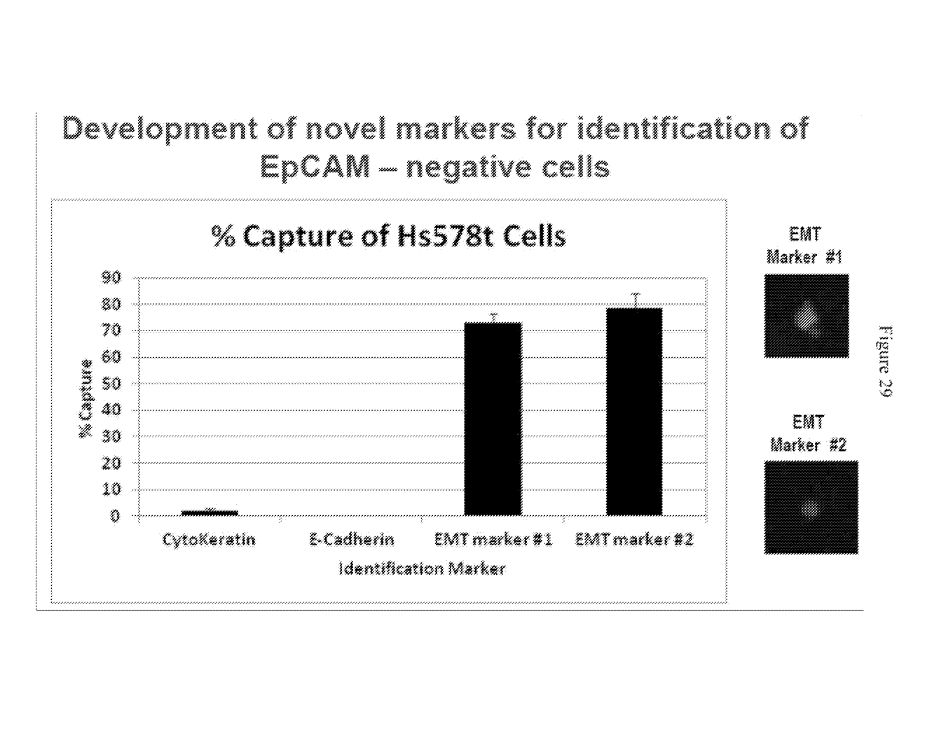

The devices and methods of the current disclosure can capture multiple populations of cells, for example EpCAM- and EpCAM+ cells, using other binding moieties to other specific markers of the various populations of cells (FIG. 29). As a non-limiting example, binding moieties specific for mesenchymal cells can be captured using binding moieties specific to mesenchymal specific cell receptors. Additionally, in some aspects, EpCAM- and EpCAM+ cells can be captured by size only and not by affinity.

### Pumping

Fluids can be driven through any of the microfluidic devices described herein either actively or passively. Fluids can be pumped using an electric field, a centrifugal field, pressure-driven fluid flow, an electro-osmotic flow, capillary action, or any combination thereof. The average direction of the fluid flow can be parallel to the walls of the channel that contains the array.

The device can employ negative pressure pumping, for example, using syringe pumps, peristaltic pumps, aspirators, or vacuum pumps. The negative pressure can allow for processing of the complete volume of a clinical blood sample, without leaving unprocessed sample in the channels. Positive pressure, for example, from a syringe pump, peristaltic pump, displacement pump, column of fluid, or other fluid pump, can also be used to pump samples through a device. The loss of sample due to dead volume issues related to positive pressure pumping can be overcome by chasing the residual sample with buffer. Pumps can typically be interfaced to the device via hermetic seals, for example, using silicone gaskets.

The flow rates of fluids in parallel channels in a device can be controlled in unison or separately. Variable and differential control of the flow rates in one or two or more channels can be achieved, for example, by employing a multi-channel individually controllable syringe manifold. The input channel distribution can be modified to decouple all of the parallel networks. The output can collect the output from all channels via a single manifold connected to a suction (no requirements for an airtight seal) outputting to a collection vial or to one or more other microfluidic devices. Alternately, the output from one or two or more networks can be collected separately for downstream processing. Separate inputs and outputs allow for parallel processing of multiple samples from one or two or more individuals.

Multiple strategies can be operational for sample processing with microfluidic devices where shear force can be minimized and flow velocity can optimally be maximized (FIG. 40). In one process a pneumatic pressure regulated pump can be attached to the blood source, and the blood can be pushed through the microfluidic device. An alternative methodology can also be compatible with capture and minimizing the shear forces. A second method comprises a microfluidic device where the blood can be pulled through the chip by a regulated flow pump, such as through the use of a pump described herein. Another innovation to the process can be the introduction of a mixing portion of the tubing immediately prior to the inlet port of the device. This specialized configuration of the inlet tubing can promote suspension and mixing of cells prior to entry, similar to the phenomena of microvortexing of blood.

### Fabrication

A variety of techniques can be employed to fabricate a device of the invention, and the technique employed will be selected based in part on the material of choice. Exemplary materials for fabricating the devices of the invention include glass, silicon, steel, nickel, poly(methylmethacrylate) (PMMA), polycarbonate, polystyrene, polyethylene, polyolefins, silicones (for example, poly(dimethylsiloxane)), cyclic olefin co-polymers (COC), cyclic olefin polymers (COP), silicone-on-insulator (SOI) wafers, and combinations thereof. Other materials are known in the art. Methods for fabricating channels in some of these materials are known in the art. These methods can include, photolithography (for example, stereolithography or x-ray photolithography), molding, microinjection molding, laser ablation, embossing, hat and cold embossing, silicon micromachining, wet or dry chemical etching, milling, diamond cutting, Lithographie Galvanoformung and Abformung (LIGA), and electroplating. For example, for glass, traditional silicon fabrication techniques of photolithography followed by wet (KOH) or dry etching (reactive ion etching with fluorine or other reactive gas) can be employed. Techniques such as laser micromachining can be adopted for plastic materials with high photon absorption efficiency. This technique can be suitable for lower throughput fabrication because of the serial nature of the process. For mass-produced plastic devices, thermoplastic injection molding, and compression molding can be suitable. Conventional thermoplastic injection molding used for mass-fabrication of compact discs (which preserves fidelity of features in sub-microns) can also be employed to fabricate the devices of the invention. For example, the device features can be replicated, manufactured, or molded using a blank or a glass master by any of the techniques of the current disclosure, for example, conventional photolithography. The glass master can be electroformed to yield a tough, thermal shock resistant, thermally conductive, hard mold. A mold, i.e. a molded blank, can serve as the master template for injection molding or compression molding the features into a plastic device. Depending on the material used to fabricate the devices and the requirements on optical quality and throughput of the finished product, compression molding or injection molding can be chosen as the method of manufacture.

Compression molding (also called hot embossing or relief imprinting) can have the advantages of being compatible with high-molecular weight polymers, which can be excellent for small structures, but can be difficult to use in replicating high aspect ratio structures and can have longer cycle times. Injection molding can work well for high-aspect ratio structures but can be most suitable for low molecular weight polymers.

A device can be fabricated in one or more pieces that can then be assembled. Separate layers of the device can contain channels for a single fluid. Layers of a device can be bonded together by clamps, adhesives, heat, anodic bonding, exposure to UV light, UV/ozone treatment, resistive heating, induction welding, solvent bonding, laser welding techniques or reactions between surface groups (for example, wafer bonding). Alternatively, a device with channels in more than one plane can be fabricated as a single piece, for example, using stereolithography or other three-dimensional fabrication techniques.

The device can be optically transparent, or have transparent windows, for visualization of cells before, during, or after flow through the device. The top and bottom surfaces of the device can be parallel to each other. The obstacles can be either part of the bottom or the top surface and can define the height of the flow channel. It can also be possible for a fraction of the obstacles to be positioned on the bottom surface, and the remainder on the top surface. The obstacles can contact both the top and bottom of the chamber, or there can be a gap between an obstacle and one surface. The obstacles can be coated with a binding moiety, for example, an antibody, a charged polymer, a molecule that binds to a cell surface receptor, an oligonucleotide or polypeptide, a viral or bacterial protein, a nucleic acid, or a carbohydrate, that can bind a population of cells in a mixture, for example, those expressing a specific surface molecule. Other binding moieties that can be used that are specific for a particular type of cell or particle are known in the art. The obstacles can be fabricated from a material to which a specific type of cell binds. Non-limiting examples of such materials can include organic polymers (charged or uncharged) and carbohydrates. Once a binding moiety, linker, or any combination thereof is coupled to the obstacles, a coating, as described herein, can also be applied to any exposed surface of the obstacles to prevent non-specific adhesion of cells to the obstacles.

The enrichment devices described herein can also include a lid that can be optionally detachable, optically transparent, clear, or optically opaque. Moreover, the base layer or sides of the device or the array of obstacles can also be optically transparent. This can allow for optical detection means positioned adjacent to or above the array of obstacles to analyze cells retained within the array. Use of a clear lid can allow visualization of detectable moieties bound to cells or particles in the device. Lids of any of the microfluidic devices can be sealed to a device or can be removable. For example, when cells are to be cultured following capture in a device, the lid can be removed prior to culturing cells in the device or following removal of target cells from the device using methods described elsewhere and herein. The lid can be made from plastic, tape, glass or any other conventional material. The device can also comprise a seal. A seal can be composed of at least one of an adhesive, a latch, or a heat-formed connection. A seal can be utilized for subsequent capturing of the cells or analysis or enumeration/visualization of the cells in the device. Thus, preferably a device has a detachable, transparent lid, a seal, and an optically transparent base layer and array of obstacles.

### Support Pillars

It is an object of the present disclosure that any of the described microfluidic devices can comprise an input, an output, and an array of obstacles disposed there-between and further comprise one or more support pillars in an array. The support pillar array can be dense enough to provide structural support and prevent large air gaps, but may not be so dense as to be equivalent to the region of the device where cell capture occurs. The support pillars can extend from the region from the inlet to the start of the array (plenum) without leaving any large gaps (i.e. gaps less that 500-1000 μm) where air can be trapped, or the structure of the device, for example the lid, can collapse during manufacturing or pressure accumulation from sample processing. The pillars can have a lower density, and thus a lower fluidic resistance, than one or more of the array capture zones. This design can prevent high shear forces in this region since the pillars occupy a portion of the chamber (for example, the plenum), that may not be the maximum width of the device, so the cells can move faster through the area containing the support pillars than they do in the capture zone array.

The support pillars can be larger than the largest obstacle in the array within the capture zone. Each of the support pillars can have a diameter of at least about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 microns and a center-to-center spacing of at least about 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 microns.

Each of the support pillars can have a diameter of at least about 30, 35, 40, 45, 50, 55, 60, 70, 80, 90, 100, 110, 120, 130, 140, 150, 160, 170, 180, 190, 200, 210, 220, 230, 240, 250, 260, 270, 280, 290, or 300 microns and a center-to-center spacing of at least about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 microns.

Each of the support pillars can have a diameter of at least about 60, 70, 80, 90, 100, 110, 120, 130, 140, 150, 160, 170, 180, 190, 200, 210, 220, 230, 240, 250, 260, 270, 280, 290, or 300 microns and can be spaced less than about 1000, 950, 900, 850, 800, 750, 700, 650, 600, 550, or 500 microns away from the input. The support pillars can be less than about 450, 400, 350, 300, 250, 200, 150, 100 or 50 microns from the input.

The arrangement of the support pillars can vary depending on the application of the devices' use or the arrangement of the obstacles within one or more of the capture zone(s). The support pillars can have a different pattern than the obstacles arrayed in the array, or capture zone of the devices. The support pillars can have a similar pattern as the obstacles arrayed in the capture zone of the devices. For example, the support pillars can be patterned in an ordered array, a random array, a square array, a triangular array, a staggered array, or a rectangular array.

The support pillars can be spaced at least about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 microns from one or more other support pillars or at a distance of at least about 50%, 55%, 60%, 65%, 70%, 75%, 80%, 85%, 90%, 95%, or 100% larger than any distance between the obstacles in the array within the capture zone.

### Plenum Shape

The regions of any of the microfluidic devices of the current disclosure, which can be either between the input and the beginning of the capture region or between the end of the capture region and the output, can be defined as the plenum, and can vary in shape. For example, the shape of these regions can be rounded, square, rectangular, triangular, or any other shape. The plenum shape can be designed to help spread out the flow from the inlet port to the full width of the array such that the cells can be distributed uniformly across the width of the device. The plenum shape can be designed to aid in priming the device so that the device can fill while minimizing air pockets formation and retention. The plenum area can be occupied by the support pillars so that the lid can be supported during assembly and sample processing. These support pillars can be lower density so that the cells may not be exposed to higher shear forces as they move from the inlet (higher linear flow rates) to the capture region (lower linear flow rates).

### Obstacle Arrangements

The current disclosure provides microfluidic devices for recovering rare cells or other target biomolecules from bodily fluids or other cellular samples which incorporate at least one specifically constructed microchannel device. Such devices can be constructed using a substrate that can be formed with a channel-like flow path which incorporates a plurality of transverse fixed obstacles, or posts, in a collection region. These obstacles can be integral with the substrate and extend between the upper and lower surfaces of the channel. The obstacles can be arranged in various array patterns to disrupt straightline (laminar) flow there-through or wherein regular streamlined flow through the array can be disrupted, thereby increasing collision frequency with the posts through the collection region. The obstacles can vary in size, for example cross-sectional diameter. Binding moieties, or sequestering agents, which can be selected to capture the desired target biomolecules and thereby collect them within the collection region of the microchannel, can be attached to the surfaces of the transverse posts, throughout the plenum, or a combination thereof. Multiple microchannels can be fabricated on a single substrate, and through the use of connecting passageways and valves, integrated operations for cell separation, analysis, diagnosis, or any combination thereof can be carried out using a single apparatus. Multiple microchannel arrangements of this type can also be used for two-step or multistep purification processes, for example the separation of more than a single subpopulation of target cells from the same liquid sample, by a series of flow through upstream and downstream collection regions containing obstacles, wherein the regions can be coated with different sequestering agents.

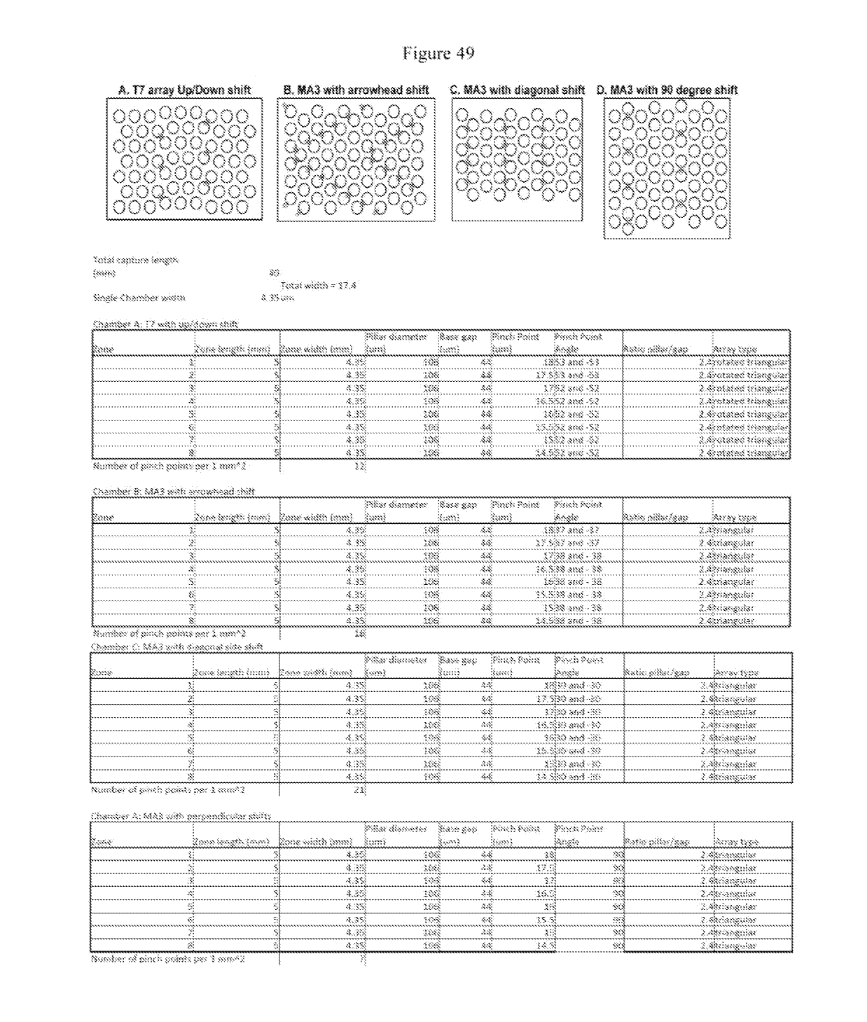

The microfluidic devices of the current disclosure feature a two-dimensional array of obstacles that can form a network of gaps, wherein the array of obstacles can be downstream of the region containing the support pillars closest to the input and comprise a plurality of rows of obstacles. Some non-limiting examples of arrays of obstacles can be seen in FIG. 49.

### T7.2

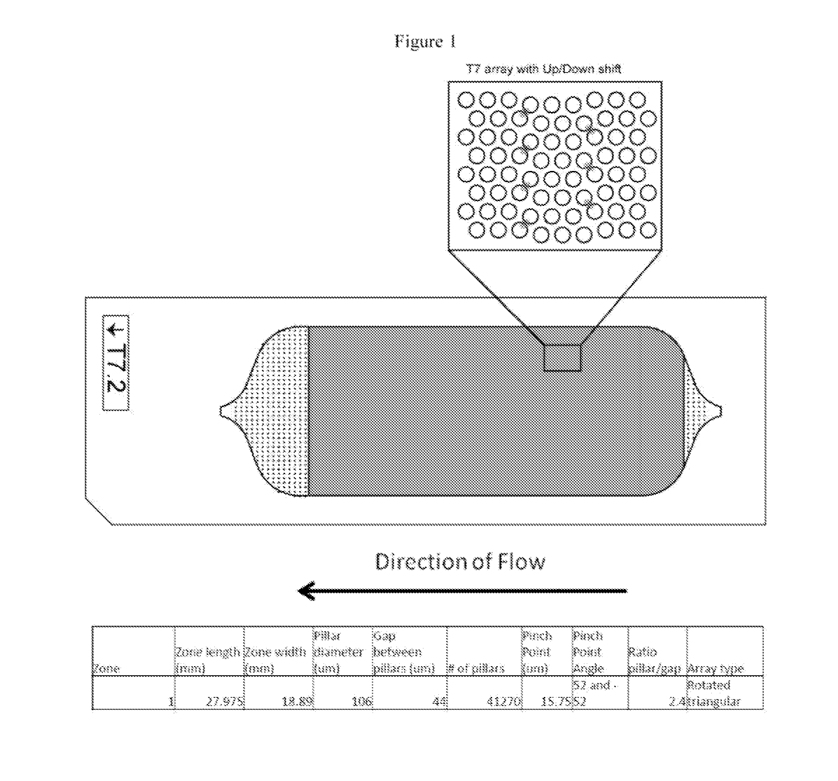

The T7.2 array design can comprise an updated plenum geometry and support pillars and pinch points or gaps created by an up and down shift in the obstacles for a distribution of gap locations (FIG. 1).

The microfluidic device can comprise a sample input, a sample output, support posts, and an array of obstacles there-between, wherein the array of obstacles comprises a first gap and a second gap, wherein the second gap can be smaller than first gap and can be situated in a repeating pattern in the array, such that the second gap occurs within every second, third, fourth, fifth, sixth, seventh, or eighth column of pillars within the array, wherein a column of obstacles can comprise all of the obstacles across the width of the microfluidic device, or perpendicular to the flow, at any one given length of the microfluidic device. The first gap distance between the obstacles can be at least about 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap distance between the obstacles can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 microns. The second gap distance can be employed during manufacturing by creating an up or down shift of one or more of the columns of obstacles such that the pillars can be placed closer to the previous column than in the standard array, therefore generating a smaller gap or pinch point. These gaps can be found at every pillar in that column and can extend across the full channel width when the entire column can be shifted, as shown in FIG. 1.

The obstacles can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

### C5.2

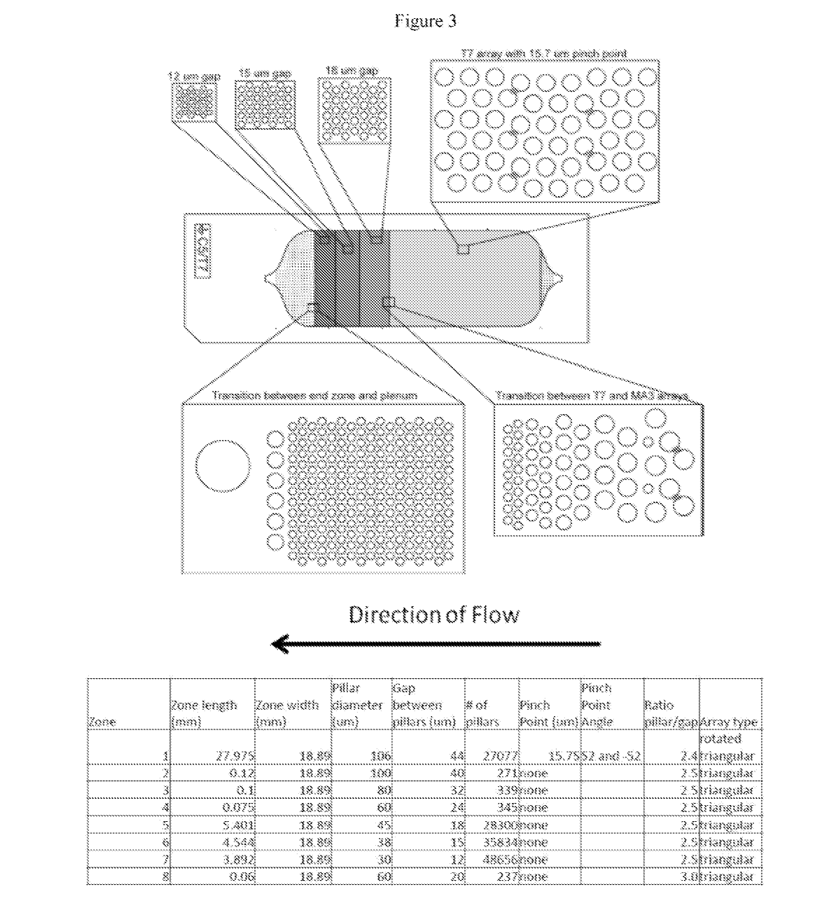

The C5.2 array design can comprise an updated plenum geometry and support pillars, a gradual transition between array regions, and can comprise pinch points or gaps created by an up and down shift, or an up shift, or a down shift in the obstacles and additional support pillars behind the obstacles of a last capture region (FIG. 3).

The microfluidic device can comprise a sample input, a sample output, support posts, and an array of obstacles there-between, wherein the array can have a plurality of regions (FIG. 3). The plurality of regions can comprise a first region comprising a first gap and a second gap between a plurality of obstacles in the first region, wherein the second gap can be smaller than first gap and can be situated in a repeating pattern in the array, such that the second gap occurs within every second, third, fourth, fifth, sixth, seventh, or eighth column of pillars within the array, wherein a column of obstacles can comprise all of the obstacles across the width of the microfluidic device, or perpendicular to the flow, at any one given length of the microfluidic device. The first gap distance between the obstacles in the first region can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap distance between the obstacles in the first region can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns.

The obstacles in the first region can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

The plurality of regions can comprise a second region having a uniform distribution of obstacles with a single gap there-between. The second region can be downstream of the first region. The plurality of regions can further comprise one or more additional regions downstream of the second region. The one or more additional regions can have a uniform distribution of obstacles with a single gap there-between, wherein the gap distance can be progressively smaller from the second region to each downstream array from the additional regions. The first, second, or subsequent gaps can be distributed in a symmetrical pattern, uniform pattern, repeating pattern, or a non-uniform pattern.

The second region can be characterized by a second gap distance between the obstacles that can be smaller than the first or second gap distance of the first region and can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The third region can be characterized by a third gap distance between the obstacles that can be smaller than the second gap distance and can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The fourth region can be characterized by a fourth gap distance between the obstacles that can be smaller than the third gap distance and can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The fifth region can be characterized by a fifth gap distance between the obstacles that can be smaller than the fourth gap distance and can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The sixth region can be characterized by a sixth gap distance between the obstacles that can be smaller than the fifth gap distance and can be at least about 8, 9, 10, 11, 12, 13, 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The seventh region can be characterized by a seventh gap distance between the obstacles that can be smaller than the sixth gap distance and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns.

The one or more additional regions can have a uniform distribution of obstacles with a single gap there-between, wherein the obstacle diameter can be uniform or different within each region and can be progressively smaller from the first or second region to each downstream array from the additional regions. For example, each of the obstacles within a region can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns, wherein the obstacle diameter can be progressively smaller from the first or second region to each downstream array from the additional regions

### T7.3

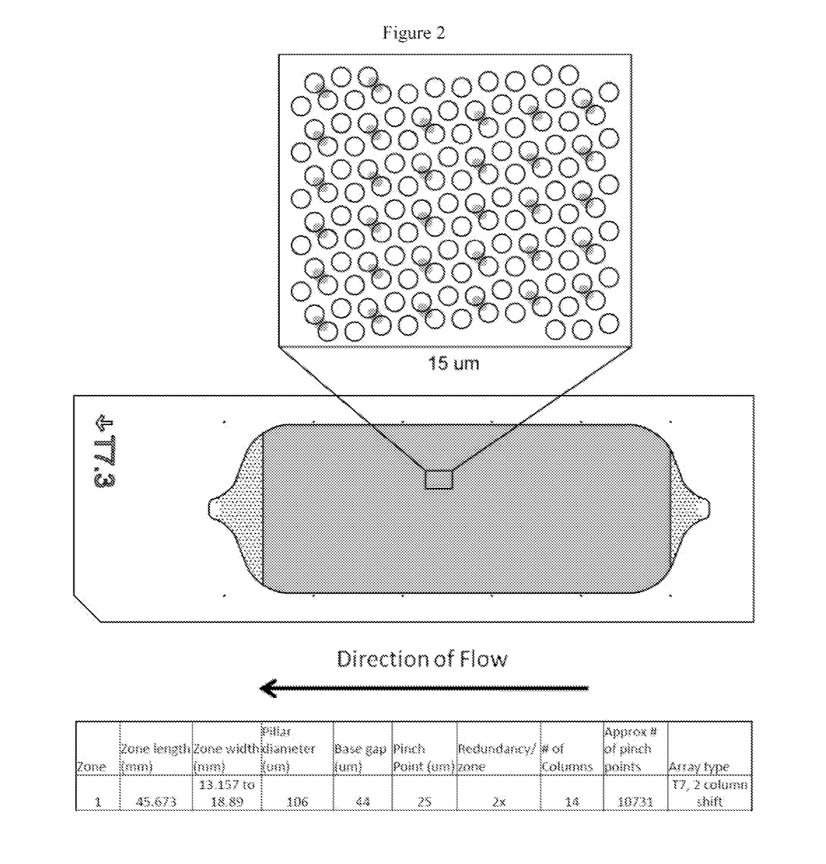

The T7.3 design can comprise an updated plenum geometry and support pillars, uniform pinch points or gaps across the array, and an increased gap density created by a reduced number of inactive obstacle columns (FIG. 2).

A microfluidic device can comprise a sample input, a sample output, and an array of obstacles there-between having a first gap between a subset of the obstacles and a second gap between a second subset of the obstacles, wherein the first gap can be larger than said second gap and wherein the second gap can be distributed across the array in a uniform, non-random pattern (FIG. 2). The second gaps can be distributed in a repeating or symmetrical pattern. The second gaps can be distributed such that the centers of the second gaps form virtual lines that traverse the flow direction. The second gap can occur within every other column of obstacles within the plenum of the array, wherein a column of obstacles can comprise all of the obstacles across the width of the microfluidic device, or perpendicular to the flow, at any one given length of the microfluidic device. The first gap distance between the obstacles can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap distance between the obstacles can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap distance can be employed during manufacturing by creating an up or down shift of one or more of the columns of obstacles such that the pillars can be placed closer to the previous column than in the standard array, therefore generating a smaller gap or pinch point. These gaps can be found at every pillar in that column and can extend across the full channel width when the entire column is shifted.

The obstacles can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

### C5.3

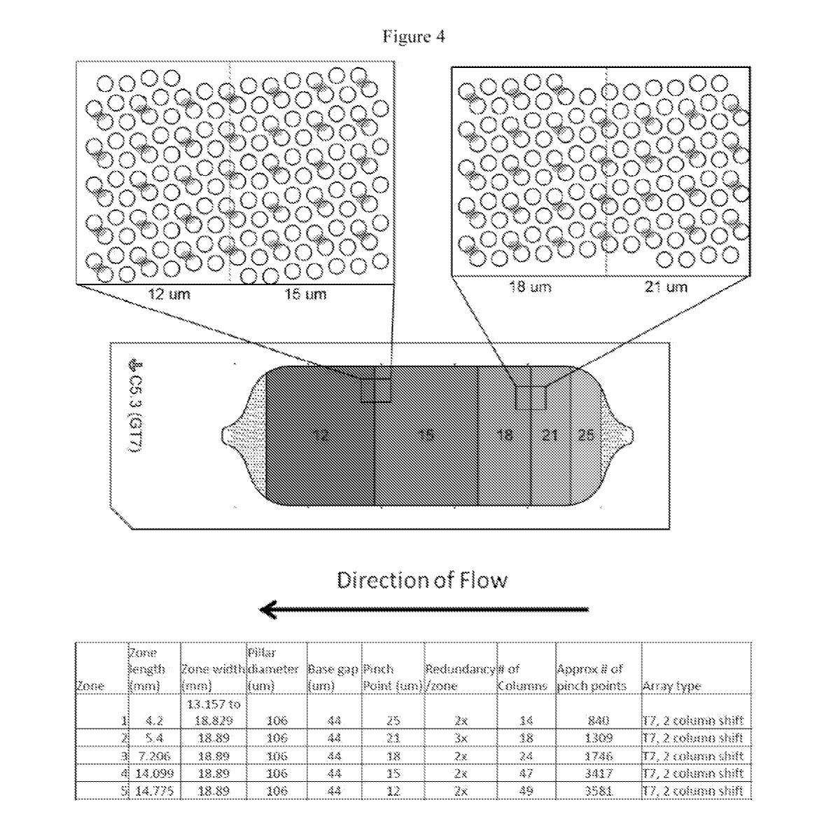

The C5.3 design can comprise an updated plenum geometry and support pillars, and a plurality of regions with two or three or four times redundancy of gaps in each region, wherein there can be lower shear forces in the gaps and lower drag forces on captured cells (FIG. 4). In one aspect, the C5.3 array can be described as a gradient T7 array.

The microfluidic device can comprise a sample input, a sample output, support posts, and an array of obstacles there-between, wherein the array can have a plurality of regions (FIG. 4). The plurality of regions can comprise a first region comprising a first gap and a second gap between a plurality of obstacles in the first region.

The first gap and the second gap can be different as described above. The plurality of regions can comprise a second region having a uniform distribution of obstacles with a first gap that can be the same as the first gap of the upstream region and a third gap, wherein the third gap can be smaller than the second gap of the upstream region there between. The second region can be downstream of the first region. The plurality of regions can further comprise one or more additional regions downstream of the first and second regions. The one or more additional regions can have a uniform distribution of obstacles with a first gap there-between, that can be the same as the first (larger) gap of the immediate upstream region, and an additional gap, wherein the additional gap distance can be progressively smaller from the second (smaller) gap of the immediate upstream region, to each downstream array from the additional regions as shown in FIG. 4. The first, second, or subsequent gaps can be distributed in a symmetrical pattern, uniform pattern, repeating pattern, or a non-uniform pattern.

The one or more additional regions can have a uniform distribution of obstacles with a single gap there-between, wherein the obstacle diameter can be uniform within each region and can be progressively smaller from the first or second region to each downstream array from the additional regions. For example, each of the obstacles within a region can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

The plurality of regions can comprise two or more regions, each with a uniform distribution of obstacles with a first and second gap there-between, wherein the second of the two gap distances can be progressively smaller from the second of the two gap distances in first region to each downstream array from the additional regions. The first gap distance between the obstacles in all of the regions can be uniform across all of the regions and can be at least about 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap distance between the obstacles in the first region can be at least about 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, or 44 microns. The second gap distance can be employed during manufacturing by creating an up or down shift of one or more of the columns of obstacles such that the pillars can be placed closer to the previous column than in the standard array, therefore generating a smaller gap or pinch point. These gaps can be found at every pillar in that column and can extend across the full channel width when the entire column is shifted.

The second gap of the second region can be characterized by a third gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap of the third region can be characterized by a fourth gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap of the fourth region can be characterized by a fifth gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap of the fifth region can be characterized by a sixth gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap of the sixth region can be characterized by a seventh gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns. The second gap of the seventh region can be characterized by a eighth gap distance between every other column of obstacles that can be smaller than the second (smaller) gap distance of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 microns.

The obstacles comprising the second, smaller gap can have an angle of attack. The angle of attack can be the angle of the gap relative the flow direction. The angle of attack can change as the gap size increases or decreases, for example, the angle of attack can become larger or smaller as the gap size increases or decreases. The angle of attack can be 90° or less than 90°, 89°, 88°, 87°, 86°, 85°, 84°, 83°, 82°, 81°, 80°, 79°, 78°, 77°, 76°, 75°, 74°, 73°, 72°, 71°, 70°, 69°, 68°, 67°, 66°, 65°, 64°, 63°, 62°, 61°, 60°, 59°, 58°, 57°, 56°, 55°, 54°, 53°, 52°, 51°, 50°, 49°, 48°, 47°, 46°, 45°, 44°, 43°, 42°, 41°, 40°, 39°, 38°, 37°, 36°, 35°, 34°, 33°, 32°, 31°, 30°, 29°, 28°, 27°, 26°, 25°, 24°, 23°, 22°, 21°, 20°, 19°, 18°, 17°, 16°, 15°, 14°, or 1° In some aspects, the angle of attack can be between about 20°-40°, 21°-40°, 22°-40°, 23°-40°, 24°-40°, 25°-40°, 26°-40°, 27°-40°, 28°-40°, 29°-40°, 20°-39°, 20°-38°, 20°-37°, 20°-36°, 20°-35°, 20°-34°, 20°-33°, 20°-32°, 20°-31°, 20°-30°, 20°-29°, 20°-28°, 20°-27°, 20°-26°, 20°-25°, 20°-24°, 20°-23°, 20°-22°, or 20°-21°. The angle of attack can be between about 30°-40°, 30°-39°, 30°-38°, 30°-37°, 30°-36°, 30°-35°, 30°-34°, 30°-33°, 30°-32°, 30°-31°, 31°-40°, 32°-40°, 33°-40°, 34°-40°, 35°-40°, 36°-40°, 37°-40°, 38°-40°, or 39°-40°.

### CS1.1

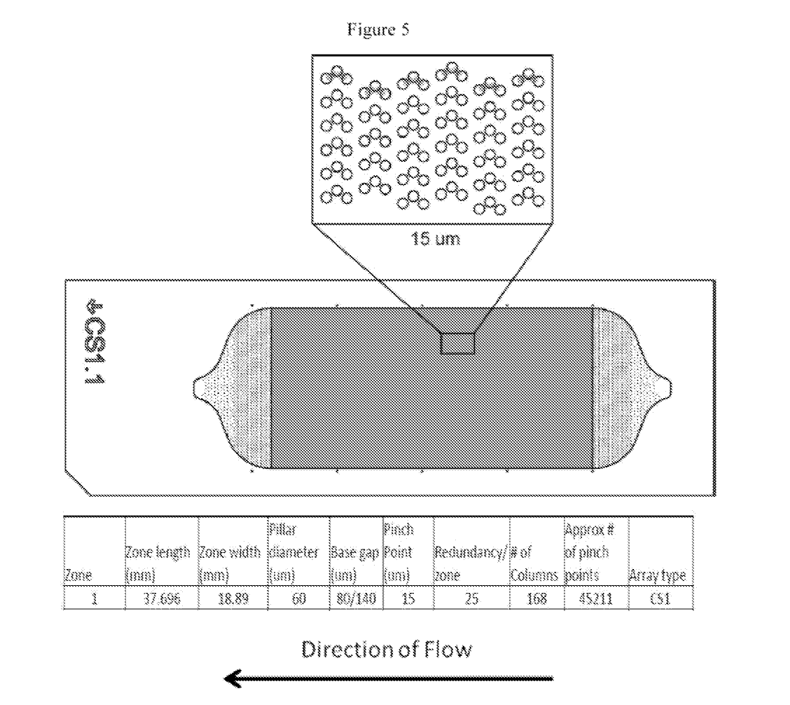

The CS1.1 design can comprise support pillars and an updated plenum geometry, wherein the support pillars can be of moderate density in the plenum to aid the priming process, and wherein the start and end of the array can be 100% of the channel width (FIG. 5).

The microfluidic device can comprise a sample input, a sample output, support posts, and an array of obstacles there-between, wherein at least a subset of the obstacles can be arranged in clusters. Substantially all or all of the obstacles can be in clusters as shown in FIG. 5. The clusters can be arranged in a non-uniform, a non-random, or a repeating pattern.

Each cluster can comprise at least three obstacles, wherein the distances between adjacent obstacles in a cluster can be smaller than distances between the cluster and its adjacent clusters. Each cluster can comprise at least three, or at least four, or at least five obstacles, wherein the distances between adjacent obstacles in a cluster can be smaller than distances between the cluster and its adjacent clusters. The distance between adjacent obstacles in a cluster can be uniform within the array. For example, the uniform distance between adjacent obstacles in a cluster can be less than about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 microns.

The largest distance between obstacles within a cluster can be at least three, four, five, six, seven, or eight fold smaller than the smallest distance between a first cluster and a second cluster adjacent to the first cluster.

The distance between a cluster and its adjacent cluster can be between about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, or 250 microns, wherein the distances between adjacent obstacles in a cluster can be smaller than distances between the cluster and its adjacent clusters.

The obstacles within the array can comprise obstacles of various sizes, for example various diameters or cross sections. Each of the obstacles within the array can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

The array can comprise at least about 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 clusters adjacent to one another.

The clusters can have a longer dimension in a first direction along a flow direction than a second direction normal to the flow direction. The clusters can have a longer dimension in a first direction normal to the flow direction than a second direction along a flow direction. The clusters can be positioned such that a first cluster can be centered upstream of a second cluster. The center of the second cluster can be off-set from center of the first cluster by an angle of 90° or less than about 90°, 89°, 88°, 87°, 86°, 85°, 84°, 83°, 82°, 81°, 80°, 79°, 78°, 77°, 76°, 75°, 74°, 73°, 72°, 71°, 70°, 69°, 68°, 67°, 66°, 65°, 64°, 63°, 62°, 61°, 60°, 59°, 58°, 57°, 56°, 55°, 540, 53°, 52°, 51°, 50°, 49°, 48°, 47°, 46°, 45°, 44°, 43°, 42°, 41°, 40°, 39°, 38°, 37°, 36°, 35°, 34°, 33°, 32°, 31°, 30°, 29°, 28°, 27°, 26°, 25°, 24°, 23°, 22°, 21°, 20°, 19°, 18°, 17°, 16°, 15°, 14°, 13°, 12°, 11°, 10°, or 1° from a horizontal line a flow direction.

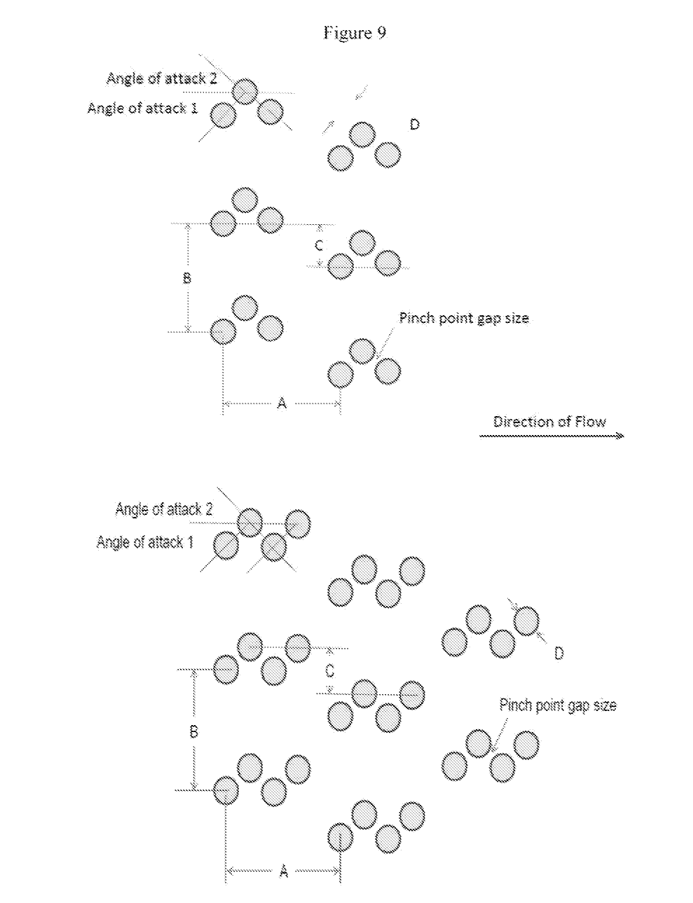

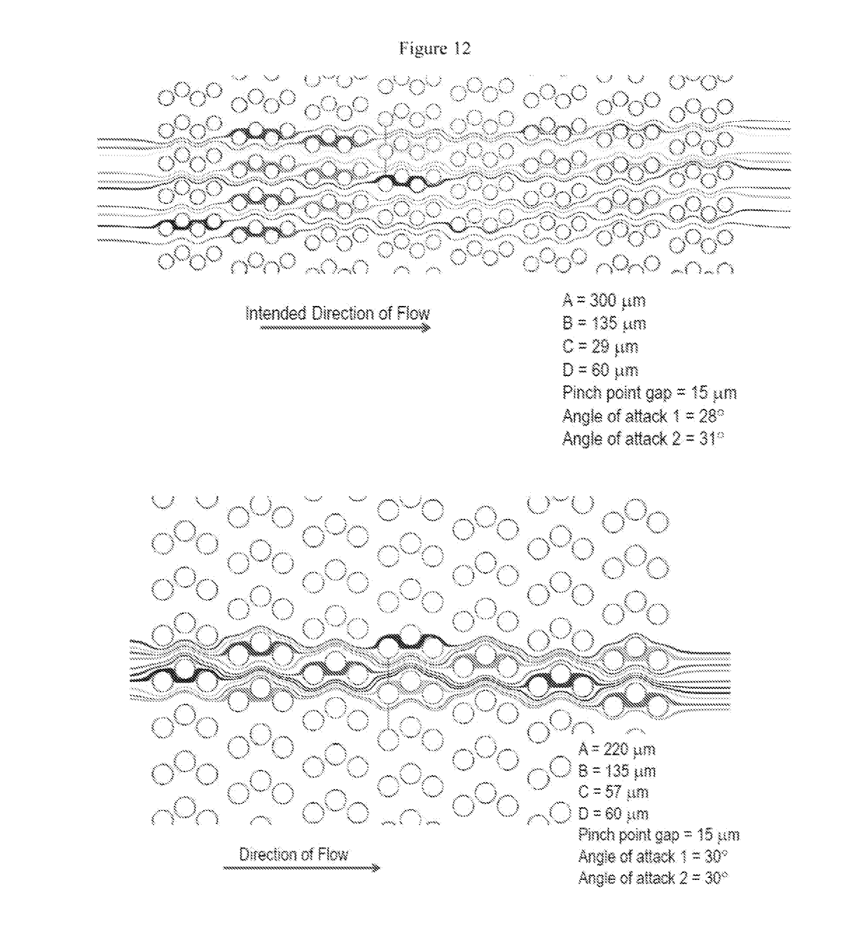

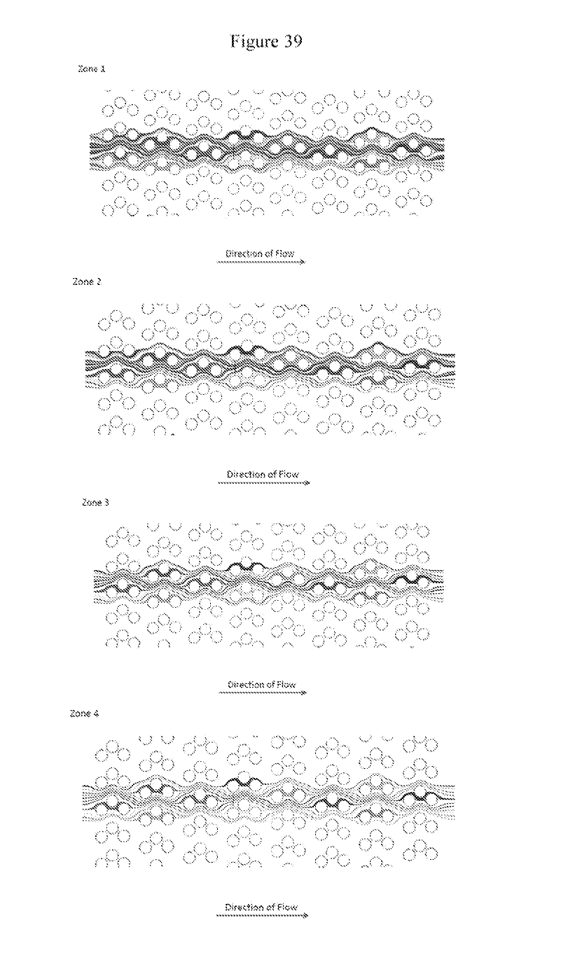

The clusters consisting of at least three, or at least four, or at least five obstacles can have first and second angles of attack. The first and second angles of attack can each be 90° or less than about 90°, 89°, 88°, 87°, 86°, 85°, 84°, 83°, 82°, 81°, 80°, 79°, 78°, 77°, 76°, 75°, 74°, 73°, 72°, 71°, 70°, 69°, 68°, 67°, 66°, 65°, 64°, 63°, 62°, 61°, 60°, 59°, 58°, 57°, 56°, 55°, 54°, 53°, 52°, 51°, 50°, 49°, 48°, 47°, 46°, 45°, 44°, 43°, 42°, 41°, 40°, 39°, 38°, 37°, 36°, 35°, 34°, 33°, 32°, 31°, 30°, 29°, 28°, 27°, 26°, 25°, 24°, 23°, 22°, 21°, 20°, 19°, 18°, 17°, 16°, 15°, 14°, 13°, 12°, 11°, 10°, or 1° In some aspects, the first and second angles of attack can each be between about 20°-40°, 21°-40°, 22°-40°, 23°-40°, 24°-40°, 25°-40°, 26°-40°, 27°-40°, 28°-40°, 29°-40°, 20°-39°, 20°-38°, 20°-37°, 20°-36°, 20°-35°, 20°-34°, 20°-33°, 20°-32°, 20°-31°, 20°-30°, 20°-29°, 20°-28°, 20°-27°, 20°-26°, 20°-25°, 20°-24°, 20°-23°, 20°-22°, or 20°-21°. The first and second angles of attack can each be between about 30°-40°, 30°-39°, 30°-38°, 30°-32°-40°, 33°-40°, 34°-40°, 35°-40°, 36°-40°, 37°-40°, 38°-40°, or 39°-40°. Examples of these descriptions and sample flow paths can be seen in FIG. 9, FIG. 12, FIG. 13, and FIG. 39.

### C5.4

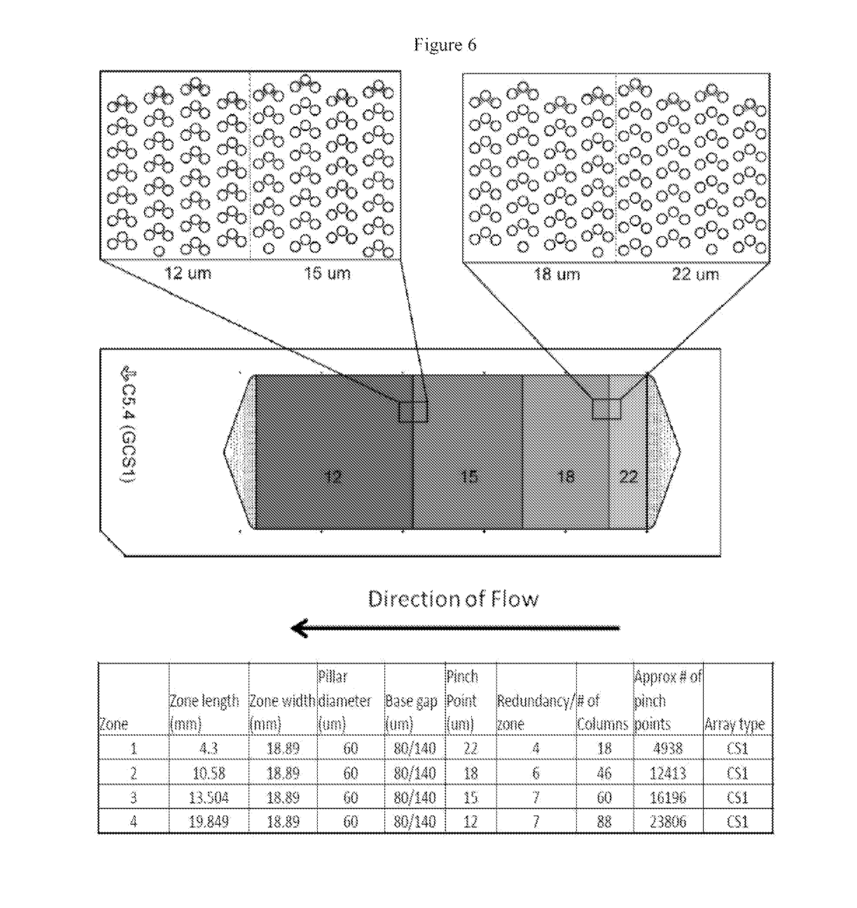

The C5.4 design can comprise an updated plenum geometry and support pillars, and a plurality of regions with four or five or six or seven or eight times redundancy of gaps in each region, wherein there can be lower shear forces in the gaps and lower drag forces on captured cells, and wherein the start and end of the array can be 100% of the channel width to increase capture length (FIG. 6).

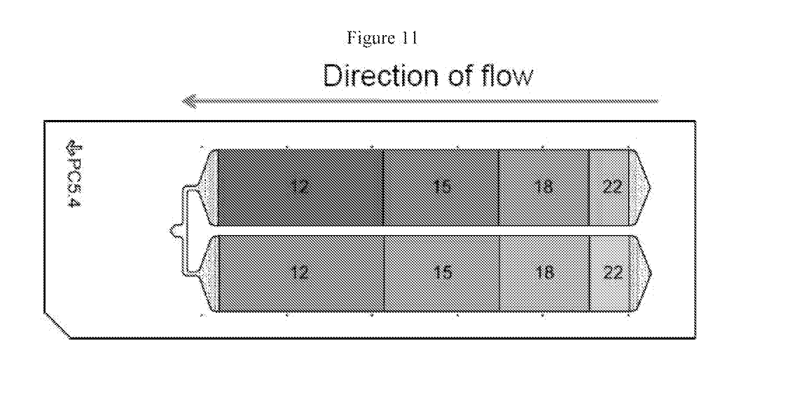

The microfluidic device can comprise a sample input, a sample output, support posts, and an array of obstacles there-between, wherein the array can comprise a plurality of regions, wherein at least a subset of the obstacles can be arranged in clusters (FIG. 6). Substantially all or all of the obstacles in one or more regions can be in clusters. The regions can be arranged in series. In one aspect, the regions can be arranged in parallel. In one aspect, the regions can be divided into two or more separate chambers or sections as shown in FIG. 11. Each chamber or the clusters of obstacles within the zones within the arrays of each chamber can have a different characteristic. For example, the clusters in each region can have pillars of varying diameter size, a different gap distance between one or more obstacles within a cluster, a different gap (spacing) distance between clusters, a different angle of attachment, a different angle between upstream or downstream clusters, a different angle between obstacles within one or more clusters, a different functionalization (for example, linkers and binding moieties) or a combination thereof.

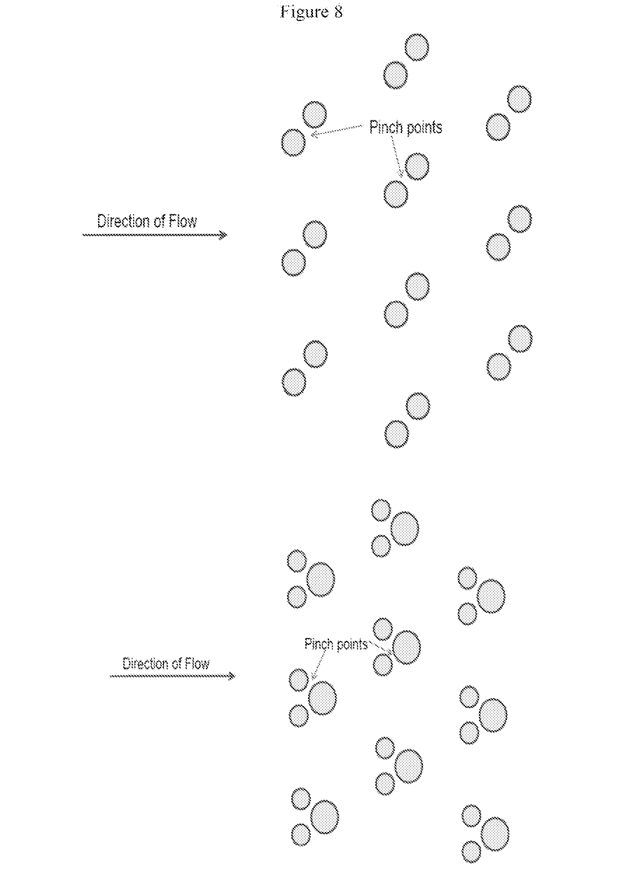

The array can comprise more than 1, 2, 3, 4, 5, 6, 7, 8, 9, or 10 regions. The clusters can be arranged in a non-uniform, a non-random, or a repeating pattern. Each cluster can comprise at least three, or at least four, or at least five obstacles. Non-limiting examples of other cluster arrangements can be seen in FIGS. 8, 9, and 10.

The obstacles within the array can comprise obstacles of various sizes, for example various diameters or cross sections. Each of the obstacles within the array can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns.

The diameter of each of the obstacles within a region of the array can be non-uniform or uniform and can be the same size or progressively smaller within each downstream array region. For example, each of the obstacles within any of the regions can have a uniform or different diameter of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns, wherein the obstacle diameter can be progressively smaller from the first or second region to each downstream array from the additional regions.

The diameter of one or more of the obstacles within a cluster of obstacles can be non-uniform. One or more of the obstacles within a cluster of obstacles can be larger or smaller than the diameter of one or more other obstacles within the cluster of obstacles as shown in FIG. 8, bottom. For example, the diameter of one or more of the obstacles within a cluster of obstacles can be at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns, and the diameter of one or more other obstacles within the cluster of obstacles can be at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 microns wherein the diameter of the other obstacles within the cluster have a larger diameter than the one or more obstacles within the same clusters.

The clusters in each region can have a different characteristic. For example, the clusters in each region can have pillars of varying diameter size, a different gap distance between one or more obstacles within a cluster, a different gap (spacing) distance between clusters, a different angle of attachment, a different angle between upstream or downstream clusters, a different angle between obstacles within one or more clusters or a combination thereof.

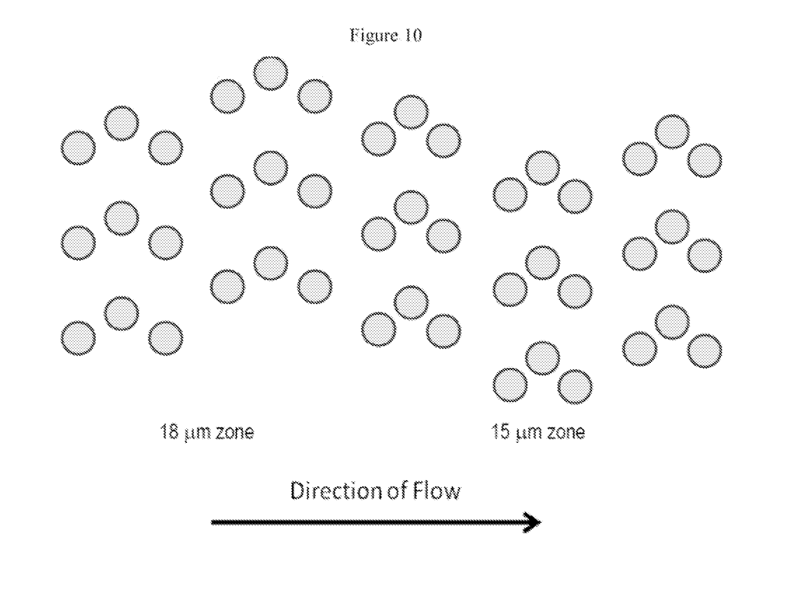

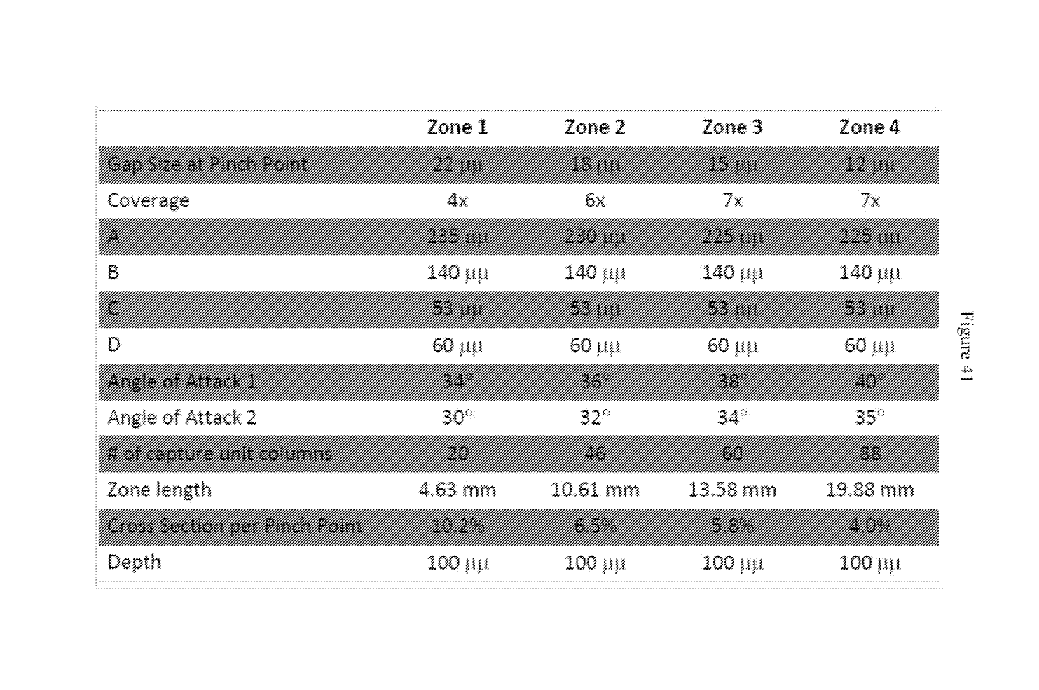

The plurality of regions can comprise 1, 2, 3, 4, 5, 6, 7, 8, 9, 10 or more regions, each with a uniform distribution of clusters of obstacles with a gap distance between one or more obstacles within a cluster, wherein the gap distance between one or more obstacles within the clusters of a region can be progressively smaller from the gap distance between one or more obstacles within the clusters of a region to each downstream region of the array from the additional regions as shown in FIG. 10 and FIG. 41.

The gap distance between the clusters of obstacles in all of the regions can be uniform across all of the regions and can be at least about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, or 250 microns.

The gap distance between the clusters of obstacles in each region can be different from the gap distance between the clusters of obstacles in any of the other regions and can be at least about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 77, 76, 77, 78, 79, 80, 81, 82, 83, 84, 88, 86, 87, 88, 89, 90, 91, 92, 93, 94, 99, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, or 250 microns, wherein the gap distance between the clusters of obstacles in a region can be the same, smaller, or greater than the gap distance between the clusters of obstacles of the region immediately upstream. For example, the gap distance between the clusters of obstacles in the first region can be 140 microns, the gap distance between the clusters of obstacles in the second region can be 130 microns, the gap distance between the clusters of obstacles in the third region can be 120 microns, the gap distance between the clusters of obstacles in the fourth region can be 110 microns, the gap distance between the clusters of obstacles in the fifth region can be 100 microns, the gap distance between the clusters of obstacles in the sixth region can be 90 microns, the gap distance between the clusters of obstacles in the seventh region can be 80 microns, and the gap distance between the clusters of obstacles in the eighth region can be 70 microns,

The gap distance between one or more obstacles within the clusters of the first region can be at least about 14, 15, 15.75, 16, 17, 18, 19, 20, 21, 22, 23, 24, 25, 26, 27, 28, 29, 30, 31, 32, 33, 34, 35, 36, 37, 38, 39, 40, 41, 42, 43, or 44 microns. The gap distance between one or more obstacles within the clusters of the second region can be smaller than the gap distance between one or more obstacles within the clusters of the region immediately upstream and can be at least about 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 microns. The gap distance between one or more obstacles within the clusters of the third region can be smaller than the gap distance between one or more obstacles within the clusters of the region immediately upstream and can be at least about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 microns. The gap distance between one or more obstacles within the clusters of the fourth region can be smaller than the gap distance between one or more obstacles within the clusters of the region immediately upstream and can be at least about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 microns. The gap distance between one or more obstacles within the clusters of the fifth region can be smaller than the gap distance between one or more obstacles within the clusters of the region immediately upstream and can be at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 microns. The gap distance between one or more obstacles within the clusters of the sixth region can be smaller than the gap distance between one or more obstacles within the clusters of the region immediately upstream and can be at least about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 microns. The gap distance between one or more obstacles within the clusters of the seventh region can be smaller than the gap distance between one or more obstacles within the clusters of the region immediately upstream and can be at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 microns.

The clusters can have a longer dimension in a first direction along a flow direction than a second direction normal to the flow direction. The clusters can have a longer dimension in a first direction normal to the flow direction than a second direction along a flow direction. The clusters can be positioned such that a first cluster can be centered upstream of a second cluster. The center of the second cluster can be off-set from center of the first cluster by an angle of less than 90°, 89°, 88°, 87°, 86°, 85°, 84°, 83°, 82°, 81°, 80°, 79°, 78°, 77°, 76°, 75°, 74°, 73°, 72°, 71°, 70°, 69°, 68°, 67°, 66°, 65°, 64°, 63°, 62°, 61°, 60°, 59°, 58°, 57°, 56°, 55°, 54°, 53°, 52°, 51°, 50°, 49°, 48°, 47°, 46°, 45°, 44°, 430, 42°, 41°, 40°, 39°, 38°, 37°, 36°, 35°, 34°, 33°, 32°, 31°, 30°, 29°, 28°, 27°, 26°, 25°, 24°, 23°, 22°, 21°, 20°, 19°, 18°, 17°, 16°, 15°, 14°, 13°, 12°, 11°, 10°, 9°, 8°, 7°, 6°, 5°, 4°, 3°, 2°, or 1° from a horizontal line a flow direction.

The clusters consisting of at least three, or at least four, or at least five obstacles can have first and second angles of attack. The first and second angles of attack can each be less than 90°, 89°, 88°, 87°, 86°, 85°, 84°, 83°, 82°, 81°, 80°, 79°, 78°, 77°, 76°, 75°, 74°, 73°, 72°, 71°, 70°, 69°, 68°, 67°, 66°, 65°, 64°, 63°, 62°, 61°, 60°, 59°, 58°, 57°, 56°, 55°, 54°, 53°, 52°, 51°, 50°, 49°, 48°, 47°, 46°, 45°, 44°, 43°, 42°, 41°, 40°, 39°, 38°, 37°, 36°, 35°, 34°, 33°, 32°, 31°, 30°, 29°, 28°, 27°, 26°, 25°, 24°, 23°, 22°, 21°, 20°, 19°, 18°, 17°, 16°, 15°, 14°, 13°, 12°, 11°, 10°, 9°, 8°, 7°, 6°, 5°, 4°, 3°, 2° ′, or 1°. In some aspects, the first and second angles of attack can each be between about 20°-40°, 21°-40°, 22°-40°, 23°-40°, 24°-40°, 25°-40°, 26°-40°, 27°-40°, 28°-40°, 29°-40°, 20°-39°, 20°-38°, 20°-37°, 20°-36°, 20°-35°, 20°-34°, 20°-33°, 20°-32°, 20°-31°, 20°-30°, 20°-29°, 20°-28°, 20°-27°, 20°-26°, 20°-25°, 20°-24°, 20°-23°, 20°-22°, or 20°-21°. The first and second angles of attack can each be between about 30°-40°, 30°-39°, 30°-38°, 30°-37°, 30°-36°, 30°-35°, 30°-34°, 30°-33°, 30°-32°, 30°-31°, 31°-40°, 32°-40°, 33°-40°, 34°-40°, 35°-40°, 36°-40°, 37°-40°, 38°-40°, or 39°-40°. Examples of these descriptions and sample flow paths can be seen in FIG. 9, FIG. 12, FIG. 13, and FIG. 39.

### Transition Zone

Any of the described arrays can further comprise a transition region between a first region and a second region. The transition region (FIG. 3) can be a region wherein 2 regions or arrays comprising different obstacle size, diameter, spacing, or pattern come together, and the space between the arrays can comprise pillars arranged to make a gradual or non-gradual change from one region or array to another. The transition zone can allow for fluid movement from one region to another while minimizing or preventing air pocket formation during priming or device operation. The transition region can comprise obstacles of different sizes. The transition region can be between one region within the plenum and another region within the plenum. The transition region can be between a region of the plenum and one or more of the regions comprising the support pillars. The transition region can be between any two regions in the device. The transition region can comprise obstacles enabled for capture of cells and other particles described herein.

### Inlets/Outlets

A port refers to an opening in the device through which a fluid sample or any other fluid can enter or exit the device. A port can be of any dimensions, but preferably can be of a shape and size that allows a sample or the desired fluid or both to be dispensed into a chamber by pumping a fluid through a conduit (or tube, or tubing) or by means of a pipette, syringe, or other means of dispensing or transporting a sample.

An inlet can be a point of entrance for sample, solutions, buffers, or reagents into a fluidic chamber, such as the microfluidic device described herein. An inlet can be a port, or can be an opening in a conduit that leads, directly or indirectly, to a chamber of an automated system.

An outlet refers to an opening at which sample, sample components, reagents, liquids, or waste exit a fluidic chamber, such as the microfluidic device described herein. The sample components and reagents that leave a chamber can be waste, i.e., sample components that are not to be used further, or can be sample components or reagents to be recovered, such as, for example, reusable reagents or target cells to be further analyzed, manipulated, or captured. An outlet can be a port of a chamber such as the microfluidic device described herein, or an opening in a conduit that, directly or indirectly, leads from a chamber of an automated system.

The device can comprise multiple inlets, multiple outlets, or a combination thereof associated with a single array of obstacles and fluid sample. The device can comprise multiple inlets, multiple outlets, or a combination thereof associated with multiple arrays of obstacles for processing a single sample, or multiple samples or both in series or in parallel or both.

The inlet and outlet of any of the microfluidic device arrays described herein can be fluidly coupled to one or more additional arrays. For example, the inlet or outlet can be fluidly coupled to one, two, three, four, five, six, seven, eight, nine, or ten additional arrays. The top layer can be made of glass and can have two slits drilled ultrasonically for inlet and outlet flows. The slit inlet/outlet dimensions can be, for example, 2 cm long and 0.5 mm wide. A manifold can then be incorporated onto the inlet/outlet slits. The inlet manifold accepts blood cells from an infusion syringe pump or any other delivery vehicle, for example, through a flexible, biocompatible tubing. Similarly the outlet manifold can be connected to a reservoir to collect the solution and cells exiting the device.

The inlet and outlet configuration and geometry can be designed in various ways. For example, circular inlets and outlets can be used. An entrance region devoid of obstacles can then be incorporated into the design to ensure that blood cells can be uniformly distributed when they reach the region where the obstacles are located. Similarly, the outlet can be designed with an exit region devoid of obstacles to collect the exiting cells uniformly without damage.

The enrichment devices herein can also include one or more inlet ports and one or more outlet ports. A port can be any region used for delivering fluid to or removing fluid from an enrichment module, such as an array of obstacles. Inlets or inlet ports refer to modules or opening that can be used for delivering fluid to an enrichment module. Outlets or outlet ports refer to modules or opening that can be used for removing fluid from an enrichment module. The device can include an inlet and an outlet, and a region of obstacles with flow path widths equal to or smaller than the second width can surround the outlet.

### Surface Chemistry

Conventionally, techniques for immobilizing a protein on a support such as a plate involve physically adsorbing a protein on a poly-L-lysine surface, or immobilizing a protein by preparing a base material by introducing an aldehyde, carboxyl, or epoxy group to a surface of glass, silicon, plastic or the like and then reacting such functional groups with an amino group of the protein. The former method has disadvantages: the protein is easily peeled off from the substrate because of the weak force of physical adsorption; higher background noises due to high nonspecific adsorption. The latter method also has disadvantages: although the use of covalent bond eliminates the peeling of immobilized protein, the process involves harmful reagents and the immobilized protein usually experiences damage in structure and function; high background noise is also often observed due to high nonspecific adsorption. More importantly, these techniques were developed for protein-protein interactions and their performance in protein-cell interaction is sub-optimal (for example, steric interference with the solid support surface).

The present invention relates to a process of coating a surface and subsequently functionalizing the surface with capture agents (for example antibodies) and the use of such immobilized capture agents for affinity based enrichment of cells, particles, and other analytes from blood and other biological fluids. Capture agents can be proteins (such as antibodies) as well as nucleic acids and other chemical compounds. The process can be optimized specifically for protein-cell/particle interaction. The nonspecific adsorption can be low and the specific affinity capture of cells can be high. The process can also be designed to provide optimal steric presentation of capture agent.

The present invention can utilize the biotin-avidin interaction for part of the process and thus awards additional benefits associated with this interaction. By immobilizing avidin (or StreptAvidin, or NeutrAvidin) on the solid support, biotinylated proteins or other chemical compounds can be immobilized right before the use. This can eliminate the need to preserve biological activity of the capture agent, give the end users the flexibility to choose a capture agents, such as a specific antibody or a cocktail of different antibodies, based on their need without additional process and cost, and can provide an opportunity to gently release captured material for further analysis via the use of desthiobiotin (that can be efficiently competed out with regular biotin).

The arrays, obstacles, surfaces, or any combination thereof, of any of the microfluidic devices described herein, can be coupled to one or more binding moieties that selectively bind one or more cells or particles or one or more types of cells or particles. For example, the binding moieties can be antibodies (for example, monoclonal anti-EpCAM antibodies or fragments thereof) that selectively bind one or more epithelial cells, cancer cells, bone marrow cells, fetal cells, progenitor cells, stem cells, foam cells, mesenchymal cells, immune system cells, endothelial cells, endometrial cells, connective tissue cells, trophoblasts, bacteria, fungi, or pathogens. All of the obstacles of the device can include these binding moieties, or alternatively, a subset of the obstacles can include these binding moieties.

Binding moieties can include, but are not limited to, antibodies, antibody derivatives, proteins, peptides, peptidomimetics, peptoids, a nucleic acid (for example, DNA, RNA, PNA, or oligonucleotide), DNA and RNA aptamers, peptide aptamers, a ligand, a protein (for example a receptor, a peptide, an enzyme, an enzyme inhibitor, an enzyme substrate, an antibody, or an immunoglobulin), an antigen, a lectin, a modified protein, a modified peptide, a biogenic amine, a complex carbohydrate, a synthetic molecule, or any other forms of a molecule which bind to the cells or particles for capture to any of the microfluidic devices of the current disclosure. The antibody-based binding moieties can be any suitable form of an antibody for example, monoclonal, polyclonal, or synthetic. The antibody-based binding moieties can include any target-binding fragment of an antibody and also peptibodies, which are engineered therapeutic molecules that can bind to human drug targets and contain peptides linked to the constant domains of antibodies.

One or two or three or four or five or six or seven or eight or more different binding moieties can be on the same obstacles within an array, on different obstacles within the array, at different locations within the array, or any combination thereof. Also, two or three or four or five or six or seven or eight regions can have the same set of binding moieties, but in different concentration.

To couple a binding moiety to the surfaces of the obstacles, the substrate can be exposed to an oxygen plasma prior to surface modification to create a layer, for example a silicon dioxide layer, to which binding moieties can be attached. There are multiple techniques other than the method described above by which binding moieties can be immobilized onto the obstacles and the surfaces of the device. Simple physio-adsorption onto the surface can be used. Another approach can use self-assembled monolayers (for example, thiols on gold) that can be functionalized with various binding moieties. Additional methods can be used depending on the binding moieties being bound and the material used to fabricate the device. Surface modification methods are known in the art. In addition, certain cells can preferentially bind to the unaltered surface of a material. For example, some cells can bind preferentially to positively charged, negatively charged, hydrophilic, or hydrophobic surfaces or to chemical groups present in certain polymers. The surface of any of the devices described herein can be a plastic or a COC.

The one or more binding moieties can be attached to the enrichment device directly or indirectly. In some instances, the binding moieties (or a subset thereof) can be attached to the device via a linker or more preferably a cleavable linker. Linkers can comprise functional groups. Functional groups can include acetals, acetoxy groups, acetylides, acid anhydrides, activating groups, acyl chlorides, acyl halides, acylals, acyloins, acylsilanes, alcohols, aldehydes, aldimines, alkanes, alkenes, alkoxides, alkyl cycloalkanes, alkyl nitritess, alkynes, allenes, amides, amidines, aminals, amines, amine oxides, azides, azines, aziridines, azoxys, bifunctionals, bisthiosemicarbazones, biurets, boronic acids, carbamates, carbazides, carbenes, carbinols, carbonate esters, carbonyls, carboxamides, carboximidates, carboxylic acids, chloroformates, cumulenes, cyanate esters, cyanimides, cyanohydrins, carbaminos, deactivating groups, depsides, diazos, diols, dithiocarbamates, enamines, enediynes, enols, enol ethers, enones, enynes, episulfides, epoxides, esters, ethers, fluorosulfonates, halohydrins, haloketones, hemiacetals, hemiaminals, hemithioacetals, hydrazides, hydroxamic acids, hydroxyls, hydroxylamines, imines, iminiums, ketenes, ketenimines, ketones, ketyls, lactams, lactols, lactones, methines, methyl groups, nitrates, nitrile ylides, nitrilimines, nitro compounds, nitroamines, nitronates, nitrones, nitronium ions, nitrosamines, nitrosos, orthoesters, osazones, oxaziridines, oximes, n-oxoammonium salts, peroxides, peroxy acids, persistent carbenes, phenols, phosphaalkenes, phosphaalkynes, phosphates, phosphinates, phosphines, phosphine oxides, phosphinites, phosphonates, phosphonites, phosphoniums, phosphoranes, s-nitrosothiols, schiff bases, selenols, selenonic acids, selones, semicarbazides, semicarbazones, silyl enol ethers, silyl ethers, sulfenamides, sulfenic acids, sulfenyl chlorides, sulfides, sulfilimines, sulfinamides, sulfenic acids, sulfite esters, sulfonamide (chemistry)s, sulfonanilides, sulfonates, sulfonyls, sulfonyl halides, sulfoxides, sultones, tellurols, thials, thioacetals, thioamides, thiocarbamates, thiocarboxys, thiocyanates, thioesters, thioethers, thioketals, thioketones, thiols, thiolactones, thioureas, tosylhydrazones, triazenes, triols, ureas, vanillyls, xanthates, ylides, ynolates, or any combinations thereof.

Linkers can be of different lengths and different structures, as is known in the art; see, generally, Hermanson, G. T., “Bioconjugate Techniques”, Academic Press: New York, 1996; and “Chemistry of Protein Conjugation and Cross-linking” by S. S. Wong, CRC Press, 1993, and U.S. Pat. No. 7,138,504 each of which are incorporated herein. Linking groups can have a range of structures, substituents, substitution patterns, or any combination thereof. They can, for example be derivitized with nitrogen, oxygen or sulfur containing groups which can be pendent from, or integral to, the linker group backbone. Examples include, polyethers, polyacids (polyacrylic acid, polylactic acid), polyols (for example, glycerol), polyamines (for example, spermine, spermidine) and molecules having more than one nitrogen, oxygen, or sulfur moiety (for example, 1,3-diamino-2-propanol, taurine), or any combination thereof. See, for example, Sandler et al. Organic Functional Group Preparations 2^{nd} Ed., Academic Press, Inc. San Diego 1983. A wide range of mono-, di- and bis-functionalized poly(ethyleneglycol) molecules are commercially available. See, for example, 1997-1998 Catalog, Shearwater Polymers, Inc., Huntsville, Ala. Additionally, those of skill in the art have available a great number of easily practiced, useful modification strategies within their synthetic arsenal. See, for example, Harris, Rev. Macromol. Chem. Phys., C(3), 325-373 (1985); Zalipsky et al., Eur. Polym. J., 19(12), 1177-1183 (1983); U.S. Pat. No. 5,122,614, issued Jun. 16, 1992 to Zalipsky; U.S. Pat. No. 5,650,234, issued to Dolence et al. Jul. 22, 1997, and references therein.

A wide variety of linking chemistries are available for linking molecules to a wide variety of solid or semi-solid particle support elements. It is expected that one of skill can select appropriate chemistries, depending on the intended application. A linker can attach to a solid substrate through any of a variety of chemical bonds. For example, a linker can be optionally attached to a solid substrate using carbon-carbon bonds, for example via substrates having (poly)trifluorochloroethylene surfaces, or siloxane bonds (using, for example, glass or silicon oxide as the solid substrate). Siloxane bonds with the surface of the substrate can be formed via reactions of derivatization reagents bearing trichlorosilyl or trialkoxysilyl groups. The particular linking group can be selected based upon, for example, its hydrophilic/hydrophobic properties where presentation of an attached polymer in solution can be desirable. Groups which can be suitable for attachment to a linking group can include, but are not limited to, amine, hydroxyl, thiol, carboxylic acid, ester, amide, isocyanate and isothiocyanate. Other derivatizing groups include aminoalkyltrialkoxys Hanes, hydroxyalkyltrialkoxysilanes, polyethyleneglycols, polyethyleneimine, polyacrylamide, polyvinylalcohol and combinations thereof. The reactive groups on a number of siloxane functionalizing reagents can be converted to other useful functional groups using methods known in the art. See, for example, Leyden et al., Symposium on Silylated Surfaces, Gordon & Breach 1980; Arkles, Chemtech 7, 766 (1977); and Plueddemann, Silane Coupling Reagents, Plenum, N.Y., 1982. Additional starting materials and reaction schemes will be apparent to those of skill in the art (U.S. Pat. No. 6,632,655).

Aptamers, affibodies or other linkers that exhibit a high affinity for the Fc portion of certain antibodies can be used to attach antibodies or antibody fragments to a solid object (for example, U.S. Pat. No. 5,831,012).

The cell binding device can be used to deplete the outlet flow of a certain population of cells, or to capture a specific population of cells expressing a certain surface molecule or cells greater than a size determined by the one or more gap sizes of the obstacles of the microfluidic device for further analysis. The cells bound to obstacles can be removed from the chamber for further analysis of the homogeneous population of cells. This removal can be achieved by incorporating one or more additional inlets and exits orthogonal to the flow direction. Cells can be removed from the chamber by purging the chamber at an increased flow rate that has a higher shear force, to overcome the binding force between the cells and the obstacles. Other approaches can involve coupling binding moieties with reversible binding properties, for example, that can be actuated by pH, temperature, or electrical field. The binding moiety, or the molecule bound on the surface of the cells, can also be cleaved by enzymatic or other chemical means.

A variety of cleavable linkers, including acid cleavable linkers, light or “photo” cleavable linkers, and enzyme cleavable linkers and the like are known in the art. Immobilization of assay components in an array can typically be via a cleavable linker group, for example, a photolabile, acid or base labile linker group. Accordingly, a cell can be released from the device or the array of obstacles, for example, by exposure to a releasing agent such as light, acid, base or the like prior to flowing the cell to an output means. Typically, linking groups can be used to attach polymers or other assay components during the synthesis of the device. Thus, linkers can operate well under organic or aqueous conditions, or a combination thereof, but cleave readily under specific cleavage conditions. The linker can, optionally, be provided with a spacer having active cleavable sites. Linking groups which facilitate polymer synthesis on solid supports and which provide other advantageous properties for biological assays are known. The linker provides for a cleavable function by way of, for example, exposure to an acid or base. Additionally, the linkers optionally have an active site on one end opposite the attachment of the linker to a solid substrate in the array. The active sites can be optionally protected during polymer synthesis using protecting groups. Among a wide variety of protecting groups which can be useful are nitroveratryl (NV OC) a-methylnitroveratryl (Menvoc), allyloxycarbonyl (ALLOC), fluorenylmethoxycarbonyl (FMOC), cc-methylnitro-piperonyloxycarbonyl (MeNPOC), —NH-FMOC groups, t-butyl esters, t-butyl ethers, and the like. Various exemplary protecting groups are described in, for example, Atherton et al., (1989) Solid Phase Peptide Synthesis, IRL Press, and Greene, et al. (1991) Protective Groups In Organic Chemistry, 2nd Ed., John Wiley & Sons, New York, N.Y. Coupling chemistries for coupling materials to the particles of the invention can be light-controllable, i.e., utilize photo-reactive chemistries. The use of photo-reactive chemistries and masking strategies to activate coupling of molecules to substrates, as well as other photo-reactive chemistries is generally known (for example, for coupling bio-polymers to solid phase materials). The use of photo-cleavable protecting groups and photo-masking permits type switching of fixed array members, i.e., by altering the presence of substrates present on a device (i.e., in response to light) (U.S. Pat. No. 6,632,655).

The cleavable linker can comprise at least one of biotin/avidin, biotin/StreptAvidin, biotin/NeutrAvidin, biotin/CaptAvidin Ig-protein A, a photo-labile linker, acid or base labile linker group, an aptamer, an affibody or other linkers that exhibit a high affinity for the Fc portion of certain antibodies can be used to attach antibodies or antibody fragments to a solid object (for example, U.S. Pat. No. 5,831,012). Any enrichment device herein can be covered with cleavable linkers comprising NeutrAvidin, avidin, CaptAvidin, or StreptAvidin protein. For example, the cleavable linker can comprise a NeutrAvidin, avidin, CaptAvidin, or StreptAvidin protein attached to the microfluidic device and a biotin-polynucleotide-anti-EpCAM moiety. Biotin can be utilized for competitive release of desthiobiotin conjugates and captured cells or particles bound thereon. Desthiobiotin can be utilized for competitive release of biotin or other biotin conjugates and captured cells or particles bound thereon. In one example an anti-EpCAM antibody such as the following: biotin-polynucleotide-anti-EpCAM moiety is attached to the enrichment device which is covered with avidin. The cleavable linker can comprise a DNA linker. An enzyme that selectively cleaves, for example a restriction enzyme, or nonspecifically cleaves, for example DNAse, a nucleic acid sequence within the nucleic acid sequence of the DNA linker can be used to release the cells, cell fragments, or particles of interest from the surface.

Surfaces of the microfluidic device, including surfaces of an array of obstacles, a lid, a port, or some combination thereof, can be coated, (for example directly or indirectly linked) or coupled to at least one or two or more binding moieties. Combinations of two or more of such agents can be immobilized upon the surfaces of the microfluidic device as a mixture of two or more entities or can be added serially. The surfaces of the microfluidic device can be treated with one or more blocking agents. For example, the surfaces of the microfluidic device can be treated with excess Ficoll or any other suitable blocking agent to reduce the retention of particles that lead to background signal when detecting one or more rare cells that can be retained by the microfluidic device.

Any of the microfluidic devices described herein can comprise an array of obstacles coated with antibodies wherein a surface of the devices has a contact angle of less than about 15°, 14°, 13°, 12°, 11°, 10°, 9°, 8°, 7°, 6°, 5°, 4°, 3°, 2°, or 1° over at least about 10, 12, 14, 16, 18, 20, 22, 24, 26, 28, 30, 32, 34, 36, 38, 40, 42, 44, 46, or 48 hours. The surface of any of the microfluidic devices described herein can be coated or functionalized with a carbohydrate. The carbohydrate can comprise dextran, dextran-hydrogel, other dextran derivatives, chitin, chitosan, alginate, cellulose, methylcellulose, HA, starch, heparin, agarose, concanavalin A, callose or laminarin, chrysolaminarin, xylan, arabinoxylan, mannan, fucoidan, galactomannan, or derivatives thereof. The carbohydrate can be at a concentration from between about 0.01%-5%, for example, 0.01%-4%, 0.01%-3.75%, 0.01%-3.5%, 0.01%-3.25%, 0.01%-3%, 0.01%-2.75%, 0.01%-2.5%, 0.01%-2.25%, 0.01%-2%, 0.01%-1.75%, 0.01%1.5%, 0.01%-1.25%, 0.01%-1%, 0.01%-0.75%, 0.01%-0.5%, 0.01%-0.25, 0.05%-2%, 0.05%1.9%, 0.05%-1.8%, 0.05%-1.7%, 0.05%-1.6%, 0.05%1.5%, 0.05%-1.4%, 0.05%1.3%, 0.05%-1.2%, 0.05%-1.1%, 0.05%-1%, 0.05%-0.9%, 0.05%-0.8%, 0.05%-0.7%, 0.05%-0.6%, 0.05%-0.5%, 0.05%-0.4%, 0.05%-0.3%, 0.05%-0.2%, or 0.05%-0.1% (w/w) on the surface.

The carbohydrate can have a molecular weight between about 1K-70K or between about 10K-70K Daltons, for example, 15K-70K, 20K-70K, 25K-70K, 30K-70K, 35K-70K, 40K-70K, 45K-70K, 50K-70K, 55K-70K, 60K-70K, 65K-70K, 10K-15K, 10K-20K, 10K-25K, 10K-30K, 10K-35K, 10K-40K, 10K-45K, 10K-50K, 10K-55K, 10K-60K, or 10K-65K Daltons. The surface can be coated with PEG. The PEG can have molecular weight of between about 1K-100K Daltons, for example, 5K-100K, 10K-100K, 15K-100K, 20K-100K, 25K-100K, 30K-100K, 35K-100K, 40K-100K, 45K-100K, 50K-100K, 55K-100K, 60K-100K, 65K-100K, 70K-100K, 75K-100K, 80K-100K, 85K-100K, 90K-100K, 95K-100K, 1K-5K, 1K-10K, 1K-15K, 1K-20K, 1K-25K, 1K-30K, 1K-35K, 1K-40K, 1K-45K, 1K-50K, 1K-55K, 1K-60K, 1K-65K, 1K-70K, 1K-75K, 1K-80K, 1K-85K, 1K-90K, 1K-95K, 5K-15K, 5K-20K, 5K-25K, 5K-30K, 5K-35K, 5K-40K, 5K-45K, 5K-50K, 5K-55K, 5K-60K, 5K-65K, 5K-70K, 5K-75K, 5K-80K, 5K-85K, 5K-90K, 5K-95K, 10K-15K, 10K-20K, 10K-25K, 10K-30K, 10K-35K, 10K-40K, 10K-45K, 10K-50K, 10K-55K, 10K-60K, 10K-65K, 10K-70K, 10K-75K, 10K-80K, 10K-85K, 10K-90K, or 10K-95K Daltons. The present invention also relates to a polymer hydrogel-coated solid support that can comprise reactive sites for attachment of PEG or bifunctional PEG. The invention further relates to use of PEG or bifunctional PEG for immobilization of proteins (antibody, avidin, StreptAvidin, CaptAvidin, NeutrAvidin) and the use of avidin, StreptAvidin, CaptAvidin, or NeutrAvidin for immobilization of biotinlyted biomolecules (for example biotinylated antibodies).

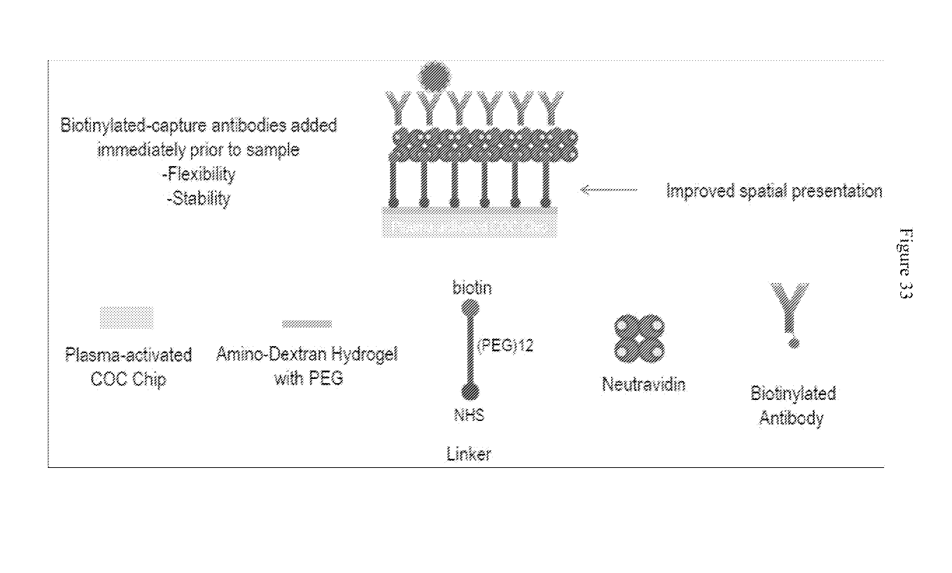

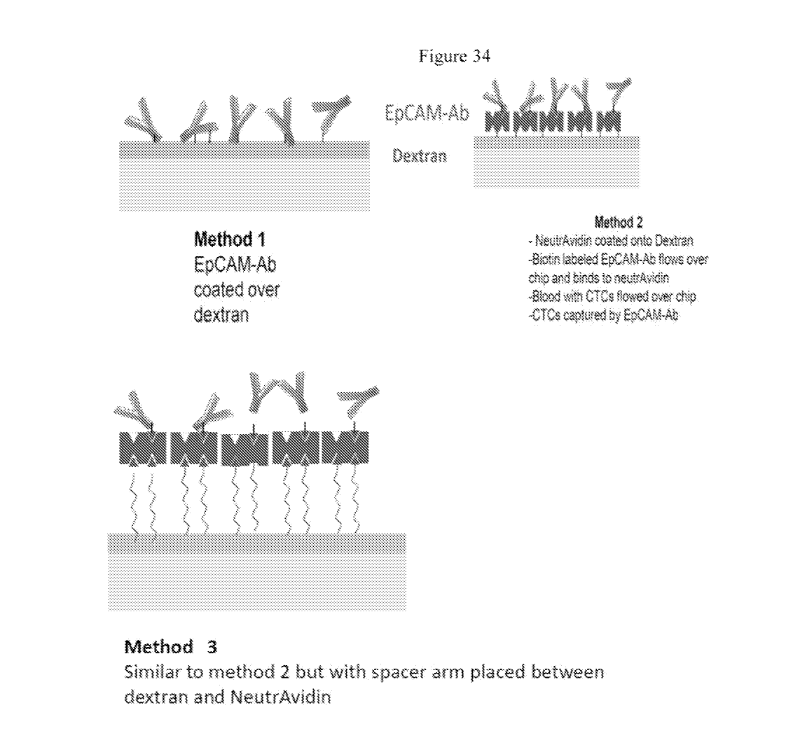

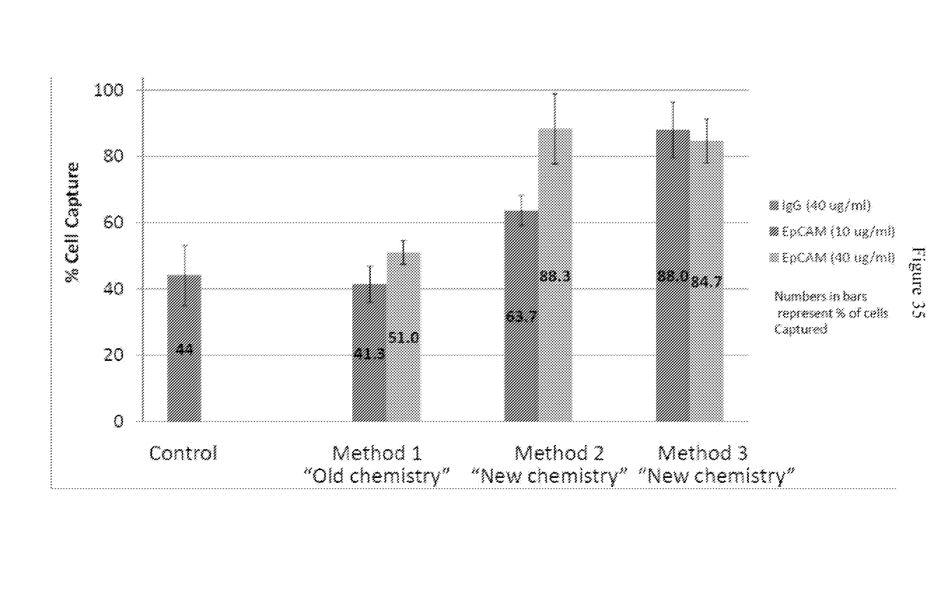

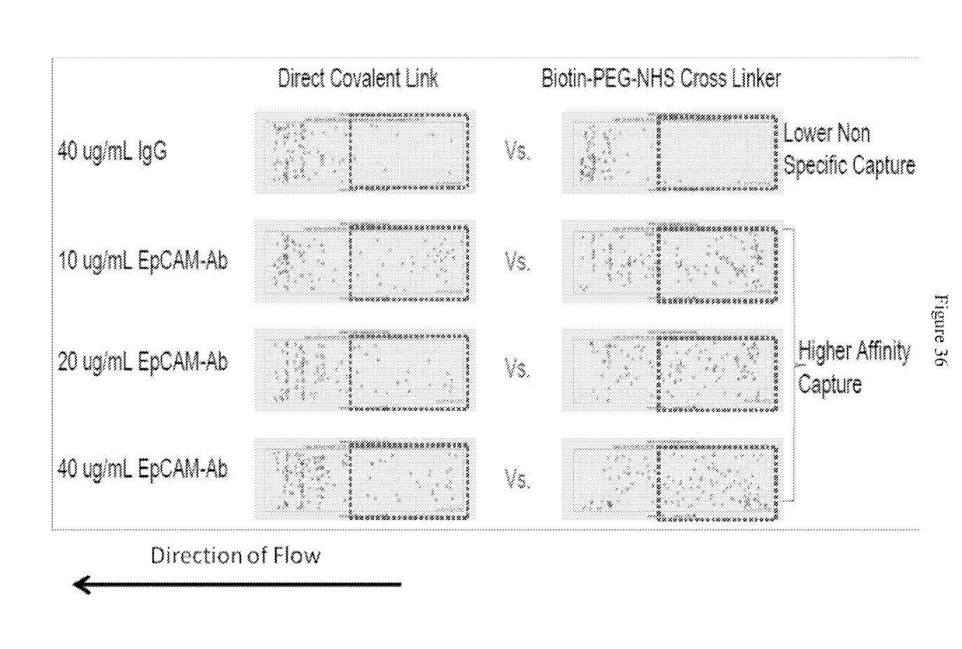

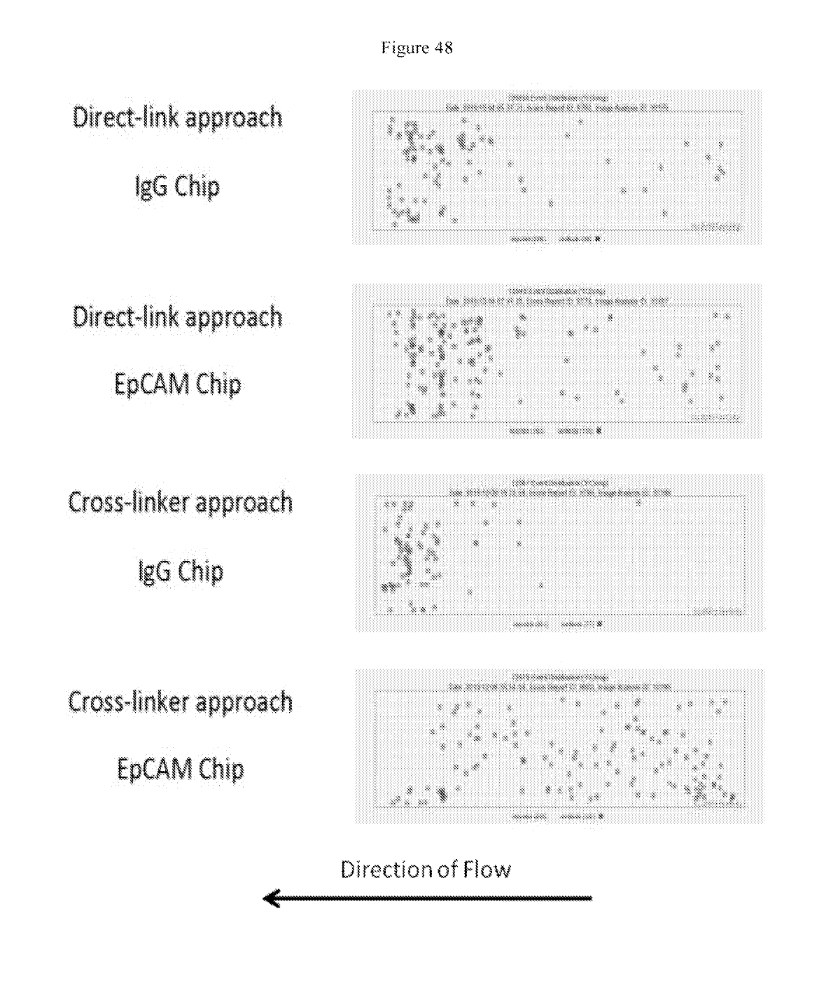

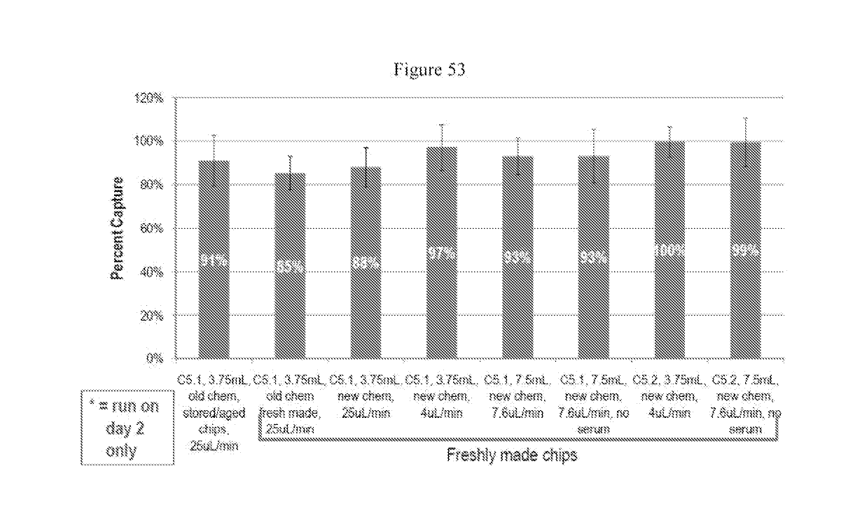

The surface can be coated with two, or three, or four, or five, or six, or seven, or eight, or more different polymers. The first polymer can be a carbohydrate and the second polymer can be polyethylene glycol (PEG), for example the first polymer can be dextran and the second polymer can be PEG. The PEG and carbohydrate can have a molar ratio of about 1:10, 2:10, 3:10, 4:10, 5:10, 6:10, 7:10, 8:10, 9:10, 10:1, 10:2, 10:3, 10:4, 10:5, 10:6, 10:7, 10:8, 10:9, or 1:1 respectively. In some aspects, the surface can further comprise a binding moiety, for example avidin, NeutrAvidin, StreptAvidin, CaptAvidin or any biotin binding protein. The amino group on NeutrAvidin can react with oxidized dextran and form a covalent double bond, which is stable for long term storage when reduced to single bond. The binding moiety can be covalently or noncovalently bound to the carbohydrate (for example dextran). The binding moiety can be bonded to the carbohydrate via a linker, for example biotin-PEG-NHS, biotin-PEG-COOH, or biotin-PEG-SH, biotin-PEG-X where X can be an amine binding group, or others described herein. Any of the microfluidic devices described herein can comprise an array of obstacles coated with avidin or an avidin derivative. As a non-limiting example, the NHS group of the Biotin-PEG-NHS cross-linker can react with amino-dextran and form a stable bond. NeutrAvidin can then bind to the biotin end of the Biotin-PEG-NHS cross linker (FIG. 33 and FIG. 34). This methodology may offer advantages from direct covalent link, for example, because NeutrAvidin links via one or more NH2 group on NeutrAvidin, thus may reduce NeutrAvidin functionality and binding efficiency. The Biotin-PEG-NHS cross linker can eliminate this factor and as a result NeutrAvidin can function better as shown in FIG. 35, FIG. 36, and FIG. 48, and FIG. 53. The length and flexibility of PEG can promote affinity binding events and rare cell capture by minimizing steric hindrances and reduce non-specific binding events. Non-limiting examples of surface coating functionalities and methods are depicted in FIG. 34.

Any of the microfluidic devices described herein can comprise a plastic surface coupled to one or more binding moieties, for example antibodies, wherein the binding moieties can be on average more than or more than about, a PEG2 or a PEG3 length from the plastic surface.

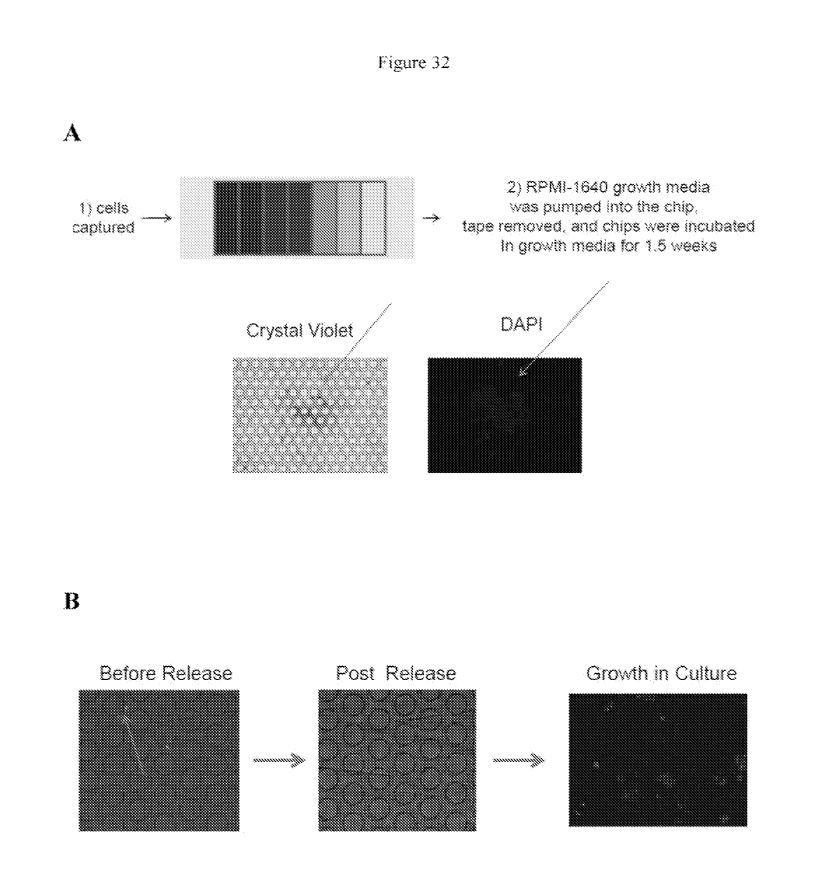

Methods for capture and release of cells, cell fragments of interest, or particles can comprise flowing a sample comprising cells, cell fragments of interest, or particles on a surface coated with carbohydrate and ligands that selectively bind a cell surface marker selectively present on the cells, cell fragments of interest, or particles and using an enzyme or chemical that selectively cleaves the carbohydrate to thereby release the cells or cell fragments of interest from the surface. For example, dextranase can be used to release cells and particles captured with a binding moiety linked to dextran as described above. Other enzymes and chemicals that selectively cleave carbohydrates can include, but are not limited to, glycosyltransferases, -glycoside hydrolases, transglycosidases, phosphorylases, lyases or acids such as periodic acid. In some aspects, cells remain viable and can be grown in culture after released by any of the methods disclosed herein (FIG. 32, bottom)

A hydrophilic linker can extend in aqueous environments and can provide maximal flexibility/solubility and activity to immobilized antibodies. Both PEG and dextran based cross-linkers can be used. In addition to high hydrophilicity as with PEG, dextran has the unique property in that it can be dissolved by dextranase under mild conditions that cause little to no damage to cells, proteins, DNAs, and RNAs. This property can be used to release capture rare species, such as CTCs, and other cancer biomarkers from the chip for advanced study.

Another option can be a hydrophilic, photo-cleavable cross-linker or just a photo-cleavable cross-linker. Both can be used for photo induced release of species from blood.

### Manufacturing

The microfluidic devices can be manufactured in a multistep manufacturing process. This process can be carried out by several key technologies. Following the formation of an etched master, the silicone molds can be fashioned. A series of customized process steps can then be executed including hot embossing, surface priming and binding moiety functionalization, antibody stabilization, input/output port assembly, and tape assembly. A reverse silicone mold or other molds can be designated and used for production runs and can be regularly replaced. The plastic microfluidic devices can be molded using a hot embossing process. Following removal from the mold, excess plastic can sheared off the flexible chip, which is then ready for functionalization. The external dimensions of the microfluidic devices can be compatible with downstream imaging.

The cell binding device can be made out of different materials. Depending on the choice of the material different fabrication techniques can also be used. The device can be made out of plastic, such as polystyrene, using a hot embossing technique. The obstacles and the other structures can be embossed into the plastic to create the bottom surface. A top layer can then be bonded to the bottom layer. Injection molding is another approach that can be used to create such a device. Soft lithography can also be utilized to create either a whole chamber made out of poly (dimethylsiloxane) (PDMS), or only the obstacles can be created in PDMS and then bonded to a glass substrate to create the closed chamber. Yet another approach involves the use of epoxy casting techniques to create the obstacles through the use of UV or temperature curable epoxy on a master that has the negative replica of the intended structure. Laser or other types of micromachining approaches can also be utilized to create the flow chamber. Other suitable polymers that can be used in the fabrication of the device can be polycarbonate, polyethylene, and poly(methyl methacrylate). In addition, metals like steel and nickel can also be used to fabricate the device of the invention, for example, by traditional metal machining. Three-dimensional fabrication techniques (for example, stereolithography) can be employed to fabricate a device in one piece. Other methods for fabrication are known in the art.

A flow device can also be created by bonding a top layer to the microfabricated silicon containing the obstacles. The top substrate can be glass to provide visual observation of cells during and after capture. Thermal bonding or a UV curable epoxy can be used to create the flow chamber. The top and bottom can also be compression fit, for example, using a silicone gasket. Such a compression fit can be reversible. Other methods of bonding (for example, wafer bonding) are known in the art. The method employed can depend on the nature of the materials used.

### Downstream Analyses

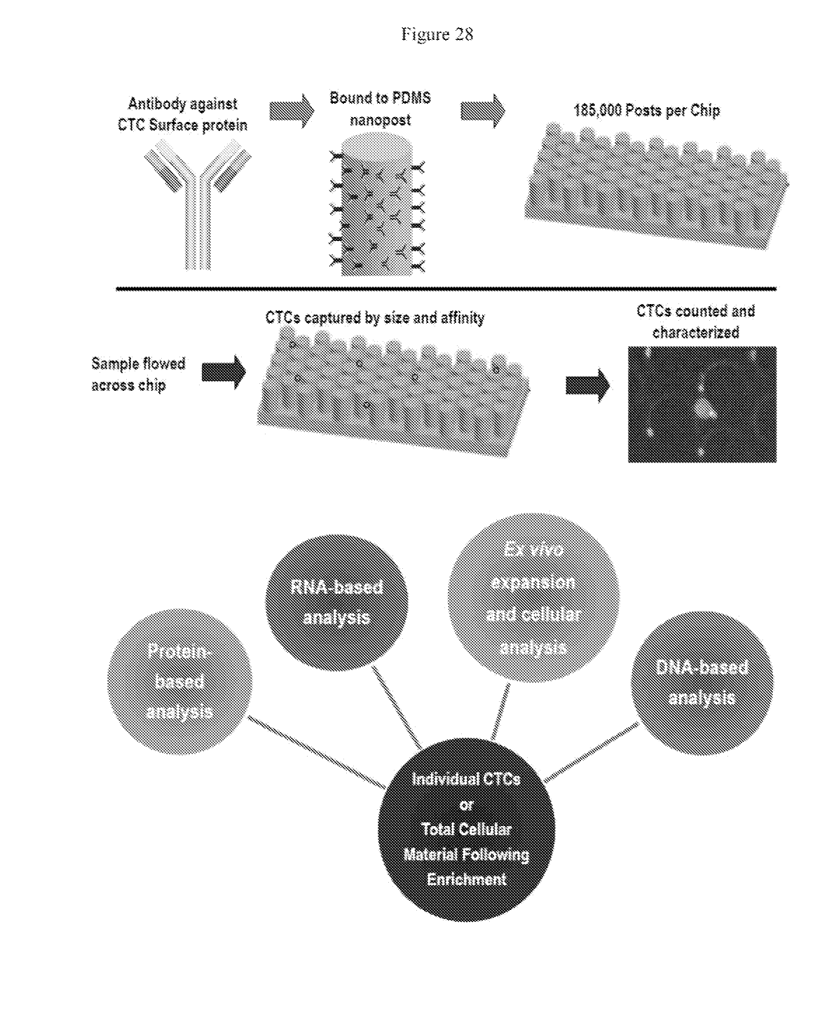

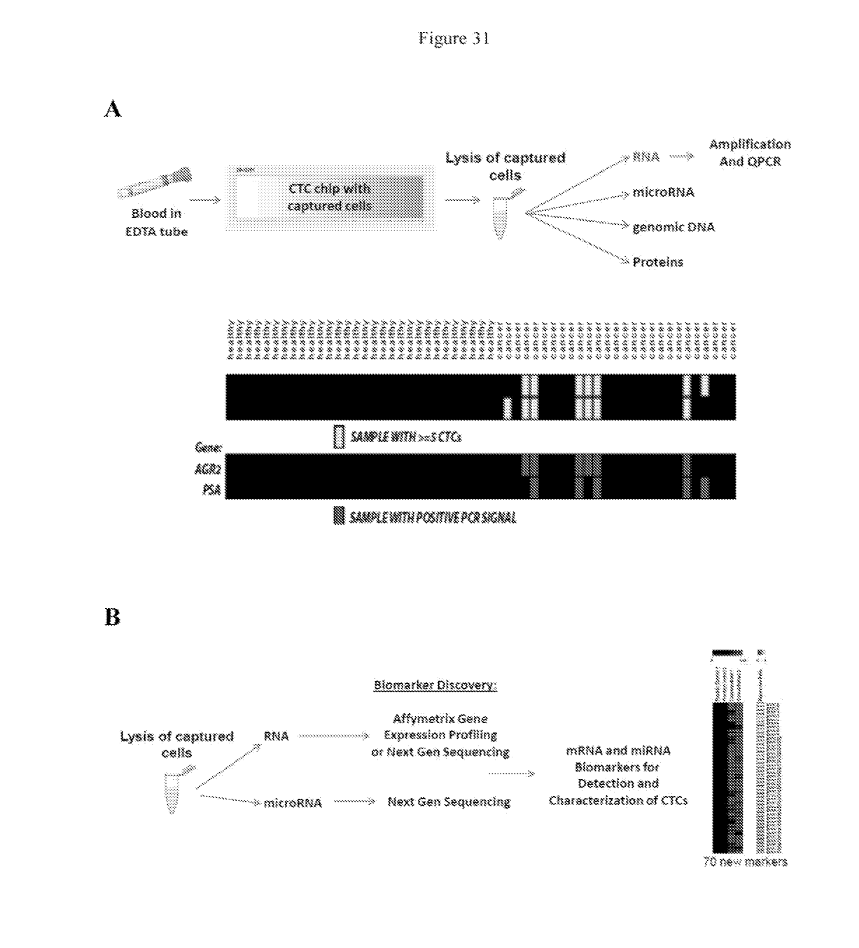

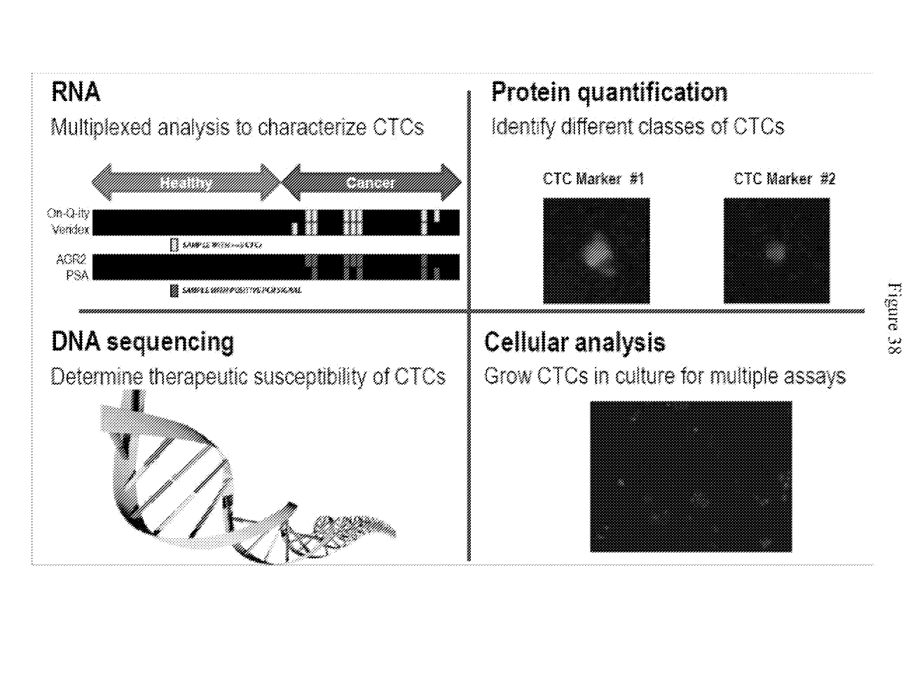

After being enriched by one or more of the devices of the invention, cells, cellular components (e.g. proteins, DNA, and RNA), cellular fragments (e.g. membranes and organelles), or other microparticles (e.g. microparticles with EpCAM containing surfaces) can be counted, collected or analyzed by various methods, for example, nucleic acid analysis (FIG. 28, FIGS. 31A and B, and FIG. 38). The sample can also be processed prior to analysis. In one non-limiting example, cells can be collected on a planar substrate for fluorescence in situ hybridization (FISH), followed by fixing of the cells and imaging. Examples of labeling reagents that can be used to label cells of interest include, but are not limited to, antibodies, quantum dots, phage, aptamers, fluorophore-containing molecules, nucleic acid binding agents, enzymes capable of carrying out a detectable chemical reaction, or functionalized beads. Generally, a labeling reagent is smaller than a cell of interest, or a cell of interest bound to a bead; thus, when a cellular sample combined with a labeling reagent is introduced to the device, free labeling reagent moves through the device undeflected and emerges from one or more outlet ports, while bound labeling reagent can be retained with the cells. Labeling of a sample prior to introduction to the device can facilitate downstream sample analysis without the need for a release step or destructive methods of analysis. Nontarget cells do not interfere with downstream sample analysis that relies on detection of the bound labeling reagent, because this reagent binds selectively to cells of interest. Detection methods of the present disclosure can be enhanced by various methods known in the art, for example, enzymatic reactions, nucleic acid hybridization, polymerase chain reaction (PCR), isothermal DNA amplification, and others.

The enrichment of one or more cells can be enhanced. For example, one or more cells can be labeled with immunoaffinity beads, thereby increasing the size of the one or more cells. In the case of epithelial cells, for example, circulating tumor cells, this can further increase their size and thus can result in more efficient enrichment. Alternatively, the size of smaller cells can be increased to the extent that they become the largest objects in solution or occupy a unique size range in comparison to the other components of the cellular sample, or so that they co-purify with other cells. The hydrodynamic size of a labeled target cell can be at least about 10%, 100%, or even 1,000% greater than the hydrodynamic size of such a cell in the absence of label. Beads can be made of polystyrene, magnetic material, or any other material that can be adhered to cells. Such beads can be neutrally buoyant so as not to disrupt the flow of labeled cells through the device of the invention.

The analysis methods can include nucleic acid analysis, protein analysis, or lipid analysis. The analysis methods can also include analysis of one or more of cell enumeration, cell morphology, pleomorphism, somatic mutation, cell adhesion, cell migration, binding, division, RNA expression, nucleic acid mutation, miRNA expression and profiling, enzymatic activity from cell lysates or within individual cells, protein expression, protein modification (for example, phosphorylation and glycosylation), mitochondrial abnormalities, cell profiling, genetic profiling, or telomerase activity or levels of a nuclear matrix protein as depicted in FIG. 28 (bottom).

Cell enumeration can result in an accurate determination of the number of target cells in the sample being analyzed. In order to produce accurate quantitative results, a surface antigen being targeted on the cells of interest typically has known or predictable expression levels and the binding of the labeling reagent should proceed in a predictable manner, free from interfering substances. Thus, methods of the invention that result in highly enriched cellular samples prior to introduction of labeling reagent can be useful. In addition, labeling reagents that allow for amplification of the signal produced can be used because of the low incidence of target cells, such as epithelial cells (for example, CTCs), in the bloodstream. Reagents that allow for signal amplification include enzymes, proteins, nucleic acids, and phage. Other labeling reagents that do not allow for convenient amplification but nevertheless produce a strong signal, such as quantum dots, can also be used in the methods of the invention.

The ratio of two cells types in the sample, for example, the ratio of cancer cells to endothelial cells, can be determined. This ratio can be a ratio of the number of each type of cell, or alternatively it can be a ratio of any measured characteristic of each type of cell.

Analysis techniques to perform the methods of analysis can include a variety of analytical techniques. A label can be used to detect a component of a cellular sample. The label can be a label conjugated to an antibody that targets any marker listed in Table 1. The label can bind to an analyte, be internalized, or be absorbed. Labels can include detectable labels and are known in the art. The detectable label can be detected based on electromagnetics, mechanical properties, electrical properties, shape, morphology, color, fluorescence, luminescence, phosphorescence, absorbance, magnetic properties, or radioactive emission or any combination thereof.

Light sensitive labels can include, as non-limiting examples, quantum dots, fluorescent dyes, or light absorbing molecules. Fluorescent dyes can include Cy dyes, Alexa dyes, or other fluorophore-containing molecules. Quantum dots, for example, Qdots® from QuantumDot Corp., can also be utilized as a label. Qdots are resistant to photobleaching and can be used in conjunction with two-photon excitation measurements. Fluorescent dyes can then be detected using a fluorometer or a fluorescent microscope. Tags specific for Surface Enhanced Resonance Raman Scattering (SERRS) can also be used. Electrical, magnetic, visual, radioactive, mechanical, and light based detection techniques are well known in the art and can be used to detect the various labels of the disclosure. Alternatively, a chromophore-containing label can be used in conjunction with a spectrometer, for example, a UV or visible spectrometer. The measurements obtained can be used to quantify the number of target cells or all cells in the sample. Alternatively, the ratio of two cell or particle types in the sample, e.g., the ratio of cancer cells to endothelial cells, can be determined. This ratio can be a ratio of the number of each type of cell or particle, or alternatively it can be a ratio of any measured characteristic of each type of cell or particle.

Physical techniques such as size filtration, density gradient centrifugation, and microscopic morphology can be used in conjunction with any of the biological or analysis techniques such as immunomagnetic isolation, flow cytometry, immunofluorescent microscopy, reverse transcriptase-polymerase chain reaction (RT-PCR), polymerase chain reaction (PCR), fluorescence microscopy, fluorescence in site hybridization (FISH), comparative genomic hybridization (CGH), PCR-based techniques, biomarker immunofluorescent staining techniques, and other techniques known in the art (reviewed in Sun et. al., Journal of Cancer Research and Clinical Oncology, 137:1151-1173 (2011)).

A label can possess covalently bound enzymes that cleave a substrate. The substrate, once cleaved, can have an altered absorbance at a given wavelength. The extent of cleavage can then be quantified, for example, with a spectrometer. Colorimetric or luminescent readouts can be possible, depending on the substrate used. A measured signal can be above a threshold of detection. The use of an enzyme label can allow for significant amplification of the measured signal and can lower the threshold of detection.

Thus, the present invention relates to kits comprising one or more of the enrichment modules herein as well as a set of labels selected from any of the labels described above. Devices can also include additional modules that can be fluidically coupled, for example, a cell counting module or a detection module. For example, the detection module can be configured to visualize an output sample of the device. Devices of the invention can process more than 20 mL of fluid per hour, or even 50 mL of fluid per hour.

Desirably, downstream analysis results in an accurate determination of the number of target cells in the sample being analyzed. In order to produce accurate quantitative results, the surface antigens being targeted on the cells of interest typically has known or predictable expression levels, and the binding of the labeling reagent should also proceed in a predictable manner, free from interfering substances. Thus, methods of the invention that result in highly enriched cellular samples prior to introduction of labeling reagent can be particularly useful. In addition, labeling reagents that allow for amplification of the signal produced are preferred, because of the low incidence of target cells, such as epithelial cells, for example, CTCs, in the bloodstream. Reagents that allow for signal amplification include enzymes and phage. Other labeling reagents that do not allow for convenient amplification but nevertheless produce a strong signal, such as quantum dots, are also desirable.

The methods of the invention allow for enrichment, quantification, and molecular biology analysis of the same set of cells. The gentle treatment of the cells in the devices of the invention, coupled with the described methods of bulk measurement, can maintain the integrity of the cells so that further analysis can be performed if desired. For example, techniques that destroy the integrity of the cells can be performed subsequent to bulk measurement; such techniques include DNA or RNA analysis, proteome analysis, or metabolome analysis. For example, the total amount of DNA or RNA in a sample can be determined; alternatively, the presence of a particular sequence or mutation, for example, a deletion, in DNA or RNA can be detected, for example, a mutation in a gene encoding a polypeptide. Furthermore, mitochondrial DNA, telomerase, or nuclear matrix proteins in the sample can be analyzed (for mitochondrial mutations in cancer, see, for example, Parrella et al., Cancer Res. 61:7623-7626 (2001), Jones et al., Cancer Res. 61:1299-1304 (2001), and Fliss et al., Science 287:2017-2019 (2000); for telomerase, see, for example, Soria et al., Clin. Cancer Res. 5:971-975 (1999)). For example, the sample can be analyzed to determine whether any mitochondrial abnormalities (see, for example, Carew et al., Mol. Cancer. 1:9 (2002), and Wallace, Science 283:1482-1488 (1999)) or perinuclear compartments are present. One useful method for analyzing DNA can be PCR, in which the cells are lysed and levels of particular DNA sequences are amplified. Such techniques can be particularly useful when the number of target cells isolated is very low. In-cell PCR can be employed; in addition, gene expression analysis (see, for example, Giordano et al., Am. J. Pathol. 159:1231-1238 (2001), and Buckhaults et al., Cancer Res. 63:4144-4149 (2003)) or fluorescence in-situ hybridization can be used, for example, to determine the tissue or tissues of origin of the cells being analyzed. A variety of cellular characteristics can be measured using any of the above techniques, such as protein phosphorylation, protein glycosylation, DNA methylation (see, for example, Das et al., J. Clin. Oncol. 22:4632-4642 (2004)), microRNA levels (see, for example, He et al., Nature 435:828-833 (2005), Lu et al., Nature 435:834-838 (2005), O'Donnell et al., Nature 435:839-843 (2005), and Calin et al., N. Engl. J. Med. 353:1793-1801 (2005)), cell morphology or other structural characteristics, for example, pleomorphisms, adhesion, migration, binding, division, level of gene expression, and presence of a somatic mutation. This analysis can be performed on any number of cells, including a single cell of interest, for example, a cancer cell. In addition, the size distribution of cells can be analyzed. Downstream analysis, for example, detection, can be performed on more than one sample, from the same subject or different subjects.

Cells found in blood are of various types and span a range of sizes. Using the methods of the invention, it can be possible to distinguish, size, and count blood cell populations, for example, CTCs. For example, a Coulter counter can be used. Under some conditions, for example, the presence of a tumor in the body that is exfoliating tumor cells, cells that are not native to blood can appear in the peripheral circulation. The ability to isolate and count large cells, or other desired cells, that can appear in the blood provides powerful opportunities for diagnosing disease states. Desirably, a Coulter counter, or other cell detector, can be fluidically coupled to an outlet of a device of the invention, and a cellular sample can be introduced to the device of the invention. Cells flowing through the outlet fluidically coupled to the Coulter counter then pass through the Coulter aperture, which includes two electrodes separated by an opening through which the cells pass, and which measures the volume displaced as each cell passes through the opening. Preferably, the Coulter counter determines the number of cells of cell volume greater than 500 fL in the enriched sample. Alternatively, the Coulter counter preferably determines the number of cells of diameter greater than 14 pm in the enriched sample. The Coulter counter, or other cell detector, can also be an integral part of a device of the invention rather than constituting a separate device. The counter can utilize any cellular characteristic, for example, impedance, light absorption; light scattering, or capacitance. In general, any means of generating a cell count can be useful in the methods of the invention. Such means include optical, such as scattering, absorption, or fluorescence means. Alternatively, non-aperture electrical means, such as determining capacitance, can be useful.

A diagnosis, prognosis, or theranosis can be made based on nucleic acid analysis on a first sample obtained from a patient and enumeration of rare cells in a second sample obtained from the patient. The first sample can be a biopsy, a blood sample, or other sample. A biopsy can be from a primary tumor or secondary tumors. The second sample can be a blood sample, or the first and second sample can be the same sample (i.e., both a blood sample). The rare cells can be CTCs and be enriched using a microfluidic device. Nucleic acid analysis can be performed on the rare cells enriched using a microfluidic device. The microfluidic device can comprise one or more binding moieties and an array of obstacles. The one or more binding moieties can comprise anti-EpCAM. Enumeration can be performed using any methods as described herein.

Nucleic acid analysis can be performed on the first blood sample, for example, a sample from a tumor, and can include RT-PCR, miRNA profiling, single nucleotide polymorphism (SNP) analysis, gene expression analysis, cDNA analysis, mRNA analysis, sequencing, genome analysis, or any combination thereof. Nucleic acid analysis can also include analysis of chromosome copy number, somatic mutations, genetic abnormalities DNA methylation, microRNA levels, or any combination thereof. RT-PCR and mRNA analysis can be performed using any method known by those skilled in the arts. Nucleic acid analysis can include analysis of genetic abnormalities. Genetic abnormalities can be detected using a label that binds a nucleic acid such as, for example, a fluorescence label or a colorimetric label. Genetic abnormalities can be detected or analyzed using FISH, in situ hybridization, SNPs, PCR or mRNA microarrays or other methods known in the art. In one non-limiting example, the method further comprises detecting genetic abnormalities in rare cells. Detection of genetic abnormalities in cells can occur in said the microfluidic device. The DNA polymorphism can be identified using a label to a unique tag sequence. In some cases, a nucleic acid tag comprises a molecular inversion probe (MIP). The methods for analyzing a nucleic acid can comprise performing one or more assays to analyze one or more nucleic acid molecules for a somatic mutation or a chromosome copy number change. A somatic mutation can include, for example, a deletion, an insertion or a point mutation. A chromosome copy number change can be an aneuploidy or a chromosome segmental aneuploidy.

The methods for analyzing a nucleic acid or modifications of nucleic acids (for example, methylation and acetylation) can comprise amplifying one or more regions of genomic DNA in a sample. In one such method, each of said one or more regions of genomic DNA can comprise one or more polymorphisms. Amplifying can be followed by, for example, ultra deep sequence analysis or quantitative genotyping (for example, using one or more MIPs). Amplifying nucleic acids can be performed using any method known to those skilled in the art. Reagents for performing nucleic acid analysis can include nucleic acids or one or more primers. The primers can be used for amplifying one or more nucleic acid sequences or can be used as a probe to a complementary nucleic acid. Nucleic acids can be used as probes to complementary nucleic acids or be used as a template for other nucleic acid methods. The nucleic acids and primers can be single-stranded, double-stranded, or conjugated to one or more functional or detectable groups. The functional groups can be detectable labels or binding moieties. The nucleic acids can include any nucleic acid or marker described herein. The primers can include portions complementary to any nucleic acid or marker described herein.

The enriched cells can then be analyzed to detect one or more subtypes of rare cells or particles or components thereof. A rare cell subtype can include any type of cell classification based on a phenotype, a genotype of the cell, or any combination thereof, including, but not limited to, circulating cancer stem cells, circulating cancer nonstem cells, tumorigenic cells, non-tumorigenic cells, apoptotic cells, non-apoptotic cells, terminal cells, non-terminal cells, proliferative cells, non-proliferative cells, cells derived from specific tissues, cells derived from specific cancer tissues, disseminated cancer cells, micrometastasized cancer cells, or cells associated with a condition. Other examples of subtypes of rare cells include those of specific tissue of origin such as circulating endothelial cells or circulating lung, liver, breast or prostate cancer cells. Other cell classifications and cell subtypes can include cells with specific cancer phenotypes. For example, breast cancer cells are known to have at least 6 different phenotypes, such as luminal/epithelial, basal/myoepithelial, mesenchymal, ErbB2, hormonal, and hereditary. Phenotypes of a cancer cell are discussed in Patent Application Publication US 2004/0191783. In some instances, the enumeration of rare cell subtype(s) by itself can be used as a diagnosis or prognosis of cancer.

Analysis of a rare cell subtype can comprise enumeration, nucleic acid analysis, protein composition analysis, etc. Enumeration can be performed using a detectable label that selectively binds to the rare cell subtype. The labeled cells can be then detected and counted using any means known in the art. A nucleic acid analysis of a rare cell subtype can include performing gene expression analysis, SNPs analysis, and ultra deep sequencing analysis on such cells.

The enumeration of the rare cell subtype(s) at two different points in time can be used to monitor treatment. For example, if the number of circulating cancer stem cell (a subtype of CTCs) increases between a first sample collected before therapy or at the beginning of treatment and a second sample collected at a later point in time (for example, after treatment), it can be concluded that the treatment is not helpful. Similarly, a baseline of circulating cancer stem cells in determined at the end of a treatment regimen and a subsequent sample obtained has an increase number of circulating cancer stern cells; there can be an indication of cancer relapse.

Rare cell subtypes, such as circulating cancer stem cells, can also be isolated using any means known in the art or described herein (for example, by flowing a sample through an array of obstacles covered with binding moieties that selectively bind the rare cell subtype, for example, anti-CD44). Enriched or isolated rare cell subtypes can be used for therapy selection or to monitor treatment by enriching rare cells from a sample from a patient, subjecting one or more rare-cell subtypes from the rare cells enriched to therapeutic agent(s), observing the effects, and determining therapy based on the effect observed. The above can be repeated over a course of a therapy to continuously monitor the efficacy of a treatment. Cancer cells can mutate during a course of treatment and the number of cells in a subtype could increase or the nucleic acid composition of a subtype could change, indicating a need to change treatment.

In some instances, enumeration of rare cell subtypes can be combined with one or more other methods described herein, such as measuring a serum marker or performing a nucleic acid analysis on a tumor biopsy. In some instances, nucleic acid analysis can be performed on the enriched or isolated rare cell subtypes. Results from such nucleic acid analysis can be combined with enumeration of rare cell subtypes to diagnose, prognose or theranose a subject. As described above, rare cells can be enriched using a microfluidic device, including any of those described herein. An analysis of a cell subtype that is a portion of one or more rare cells enriched from a sample obtained from a patient can be repeated over time for diagnosis, prognosis, or theranosis of a condition in a patient.

### Methods for Diagnosing, Prognosing, or Theranosing

The methods of the invention can comprise diagnosing, prognosing, or theranosing based on the analysis methods described herein. The methods for diagnosing, prognosing, or theranosing can comprise obtaining a sample from a patient, analyzing a sample obtained from a patient, enriching a sample obtained from a patient for one or more cells, analyzing one or more cells enriched from a sample obtained from a patient, or any combination thereof.

Diagnosing can comprise determining the condition of a patient. For example, a patient can be diagnosed with cancer or with another disease based on results from obtaining a sample from the patient, enriching a sample in one or more rare cells, and analyzing the one or more rare cells.

Prognosing can comprise determining the outcome of a patient's disease, the chance of recovery, or how the disease will progress. For example, a patient can obtain a prognosis of having a 50% chance of recovery based on results from obtaining a sample from the patient, enriching a sample in one or more rare cells, and analyzing the one or more rare cells.

Theranosis can comprise determining a therapy treatment. For example, a patient's therapy treatment can be chosen based on the response of one or more enriched cells that have been cultured and treated with a therapeutic agent.

As described herein, epithelial cells exfoliated from solid tumors have been found in the circulation of patients with cancers of the breast, colon, liver, ovary, prostate, and lung. In general, the presence of CTCs after therapy has been associated with tumor progression and spread, poor response to therapy, relapse of disease, decreased survival over a period of several years, or any combination thereof. Therefore, enumeration, characterization and analysis of CTCs can offer a means to stratify patients for baseline characteristics that predict initial risk and subsequent risk based upon response to therapy. The devices and methods of the invention can be used, for example, to evaluate cancer patients and those at risk for cancer.

In any of the methods of diagnosis described herein, either the presence or the absence of an indicator of cancer (for example, a cancer cell, particle, nucleic acid, or protein) or of any other disorder, can be used to generate a diagnosis. In one example, a blood sample can be drawn from the patient and introduced to a device of the invention with a critical size chosen appropriately to enrich epithelial cells, for example, CTCs, from other blood cells. Using a method of the invention, the number of epithelial cells in the blood sample can be determined. For example, the cells can be labeled with an antibody that binds to EpCAM, and the antibody can have a covalently bound fluorescent label. A bulk measurement can then be made of the enriched sample produced by the device, and from this measurement, the number of epithelial cells present in the initial blood sample can be determined. Microscopic techniques can be used to visually quantify the cells in order to correlate the bulk measurement with the corresponding number of labeled cells in the blood sample. Besides epithelial tumor cells, there can be other cell types that can be involved in metastatic tumor formation. Studies have provided evidence for the involvement of hematopoietic bone marrow progenitor cells and endothelial progenitor cells in metastasis (see, for example, Kaplan et al., Nature 438:820-827 (2005), and Brugger et al., Blood 83:636-640 (1994)). The number of cells of a second cell type, for example, hematopoietic bone marrow progenitor cells, for example, progenitor endothelial cells, can be determined, and the ratio of epithelial tumor cells to the number of the second cell type can be calculated. Such ratios can be of diagnostic value in selecting the appropriate therapy and in monitoring the efficacy of treatment. Cells involved in metastatic tumor formation can be detected using any methods known in the art. For example, antibodies specific for particular cell surface markers can be used. Useful endothelial cell surface markers include, but are not limited to, CD105, CD106, CD144, and CD146; useful tumor endothelial cell surface markers include, but are not limited to, TEM1, TEM5, and TEM8 (see, for example, Carson-Walter et al., Cancer 15 Res. 61:6649-6655 (2001)); and useful mesenchymal cell surface markers include, but are to limited to, CD133. Antibodies to these or other markers can be obtained from, for example, Chemicon, Abcam, and R&D Systems. By making a series of measurements, optionally made at regular intervals such as one day, two days, three days, one week, two weeks, one month, two months, three months, six months, or one year, one can track the level of epithelial cells present in’ a patient's bloodstream as a function of time. In the case of existing cancer patients, this provides a useful indication of the progression of the disease and assists medical practitioners in making appropriate therapeutic choices based on the increase, decrease, or lack of change in epithelial cells, for example, CTCs, in the patient's bloodstream. For those at risk of cancer, a sudden increase in the number of cells detected can provide an early warning that the patient has developed a tumor. This early diagnosis, coupled with subsequent therapeutic intervention, can be likely to result in an improved patient outcome in comparison to an absence of diagnostic information.

A method for monitoring for cancer recurrence can comprise enumerating or characterizing CTCs enriched from a plurality of samples derived from a patient at different points in time and enumerating and characterizing CTCs from the patient, and using the data to determine likelihood of cancer recurrence in said patience with at least 80% confidence.

Another contemplated method is for monitoring treatment efficacy in a patient receiving cancer treatment that can comprise enumerating or characterizing CTCs enriched from a sample from said patient derived before treatment and at least one sample derived after treatment, and using the data to determine whether a treatment can be efficacious with at least 80% confidence.

Another contemplated method is for screening for cancer in a patient comprising enumerating or characterizing CTCs enriched from a sample from said patient, and using the data to determine whether the patient has cancer or should seek further tests to confirm the cancer, wherein the screen can have sensitivity of at least 80%.

Any of the aforementioned methods can further comprise performing molecular analysis on CTCs captured or classifying CTCs captured, and using this information to make determinations of likelihood of cancer recurrence, whether a treatment can be efficacious, or whether a patient has cancer or should seek further tests to confirm the cancer, or any combination thereof. Any of the aforementioned methods can further comprise comparing cells captured within each of one or more of the regions or zones from any of the microfluidic devices described herein.

One or more of the devices and methods described herein can be used with a sample of at least about 1 mL or at least about 10 mL that can be processed in less than about 20 hours. For example, diagnostic, prognostic, or theranostic determinations as described above can be made with a sample of at least about 1 mL or at least about 10 mL, for example, at least about 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, or 40 mL, that can be processed in less than about 20 hours, for example in less than about 1, 2, 3, 4, 5, 6, 7, 8, 9, 10, 11, 12, 13, 14, 15, 16, 17, 18, or 19 hours. Other known microfluidic devices are not capable of providing means for such determinations with such high volumes of samples in such a short amount of time. Thus, the methods and devices described herein offer a distinct advantage over previous designs due to the quickness by which a patient can be diagnosed, prognosed, or theranosed.

Diagnostic methods can include making bulk measurements of labeled epithelial cells, for example, CTCs, isolated from blood, as well as techniques that destroy the integrity of the cells. For example, PCR can be performed on a sample in which the number of target cells isolated is very low and by using primers specific for particular cancer markers, information can be gained about the type of tumor from which the analyzed cells originated. Additionally, RNA analysis, proteome analysis, or metabolome analysis can be performed as a means of diagnosing the type or types of cancer present in the patient. For example, one important diagnostic indicator for lung cancer and other cancers can be the presence or absence of certain mutations in EGFR (see, for example, International Publication WO 2005/094357). Using the devices and method of the invention, one can monitor patients taking such drugs by taking frequent samples of blood and determining the number of epithelial cells, for example, CTCs, in each sample as a function of time. This provides information as to the course of the disease. For example, a decreasing number of circulating epithelial cells over time suggests a decrease in the severity of the disease and the size of the tumor or tumors. Following quantification of epithelial cells, these cells can be analyzed by PCR to determine what mutations can be present in the specific genes expressed in the epithelial cells. The methods of the invention described above are not limited to epithelial cells and cancer, but rather can be used to diagnose any condition. Exemplary conditions that can be diagnosed using the methods of the invention can be hematological conditions, inflammatory conditions, ischemic conditions, neoplastic conditions, infections, traumas, endometriosis, and kidney failure (see, for example, Takahashi et al., Nature Med. 5:434-438 (1999), Healy et al., Hum. Reprod. Update 4:736-740 (1998), and Gill et al., Circ. Res. 88:167-174 (2001)). Neoplastic conditions can include acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, endometrial cancer, esophageal cancer, gastric cancer, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreatic cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

A cellular sample taken from a patient can be processed through any of the devices disclosed herein in order to produce a sample enriched in any cell of interest, for example, a rare cell. Detection of this cell in the enriched sample can then enable one skilled in the art to diagnose the presence or absence of a particular condition in the patient. Furthermore, determination of ratios of numbers of cells in the sample, for example, cancer cells to endothelial cells, can be used to generate a diagnosis. Alternatively, detection and or quantification of cancer biomarkers, for example, EpCAM or any of those listed in Table 1, or a nucleic acid associated with cancer, for example, a nucleic acid encoding any marker listed in Table 1, can result in the diagnosis of a cancer or another condition. For example, analysis or quantification of the expression level or pattern of such a polypeptide or nucleic acid, for example, cell surface markers, genomic DNA, mRNA, or microRNA can result in a diagnosis.

Cell detection can be combined with other information, for example, imaging studies of the patient, in order to diagnose a patient. For example, computed axial tomography, positron emission tomography, or magnetic resonance imaging can be used. A diagnosis can also be made using a cell pattern associated with a particular condition. For example, by comparing the size distribution of cells in an enriched sample, for example, a sample containing cells having a hydrodynamic size greater than 12 microns, with a size distribution associated with a condition, for example, cancer, a diagnosis can be made based on this comparison. A cell pattern for comparison can be generated by any method. For example, an association study can be performed in which cellular samples from a plurality of control subjects (for example, 50) and a plurality of case subjects (for example, 50) having a condition of interest can be processed, for example, by enriching cells having a hydrodynamic size greater than 12 microns, the results samples can be analyzed, and the results of the analysis can be compared. To perform such a study, it can be useful to analyze RNA levels, for example, mRNA, ribosomal RNA (rRNA), snoRNA, rasiRNA, microRNA, siRNAs, long non-coding RNAs (long ncRNAs, lncRNA), and piRNA levels in the enriched cells. Alternatively, it can be useful to count the number of cells enriched in each case, or to determine a cellular size distribution, for example, by using a microscope, a cell counter, or a microarray device. The presence of particular cell types, for example, rare cells, can also be identified. Once a drug treatment is administered to a patient, it can be possible to determine the efficacy of the drug treatment using the methods of the invention. For example, a cellular sample taken from the patient before the drug treatment, as well as one or more cellular samples taken from the patient concurrently with or subsequent to the drug treatment, can be processed using the methods of the invention. By comparing the results of the analysis of each processed sample, one can determine the efficacy of the drug treatment. For example, an enrichment device can be used to enrich cells having a hydrodynamic size greater than 12 microns, or cells having a hydrodynamic size greater than or equal to 6 microns and less than or equal to 12 microns, from other cells. Any other detection or analysis described above can be performed, for example, identification of the presence or quantity of specific cell types.

### Additional Components

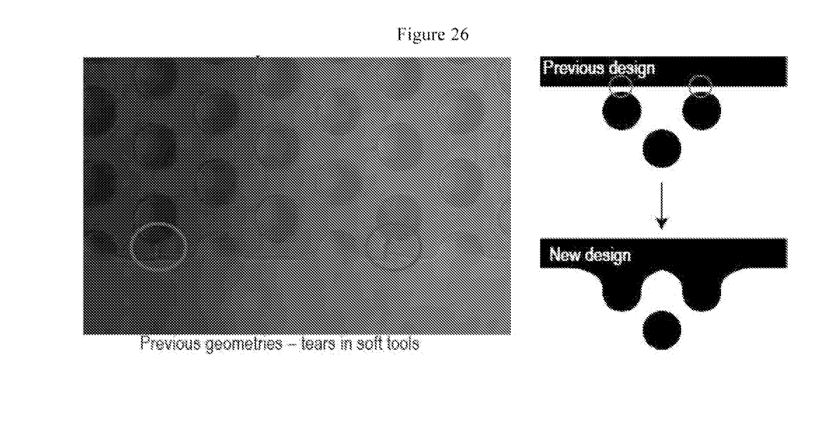

During manufacturing of a microfluidic device, the array layout can result in some obstacles close (i.e., less than 12 microns) to the edge of the channel, which can result in a soft tool that can tear. The new designs of the current disclosure can comprise arrays wherein all gaps less than 12 microns from the edge can be removed and arranged as depicted in FIG. 26.

In addition to an array of gaps, devices of the invention can include additional elements or modules, for example, for isolation, enrichment, collection, manipulation, or detection, for example, of CTCs. Such elements are known in the art. For example, devices can include one or more inlets for sample or buffer input, and one or more outlets for sample output. Arrays can also be employed on a 20 device having components for other types of enrichment or other manipulation, including affinity, magnetic, electrophoretic, centrifugal, and dielectrophoretic enrichment. Devices of the invention can also be employed with a component for two-dimensional imaging of the output from the device, for example, an array of wells or a planar surface. Preferably, arrays of gaps as described herein can be employed in conjunction with affinity enrichment. In one example, a detection module can be fluidically coupled to a separation or enrichment device of the invention. The detection module can operate using any method of detection disclosed herein, or other methods known in the art. For example, the detection module includes a microscope, a cell counter, a magnet, a biocavity laser (see, for example, Gourley et al., J. Phys. D: Appl. Phys. 36: R228-R239 (2003)), a mass spectrometer, a PCR device, an RT-PCR device, a matrix, a microarray, or a hyperspectral imaging system (see, for example, Vo-Dinh et al., IEEE Eng. Med. Biol. Mag. 23:40-49 (2004)).

A computer terminal can be connected to the detection module. For instance, the detection module can detect a label that selectively binds to cells of interest. In another example, a capture module can be fluidically coupled to a separation or enrichment device of the invention. For example, a capture module includes one or more binding moieties that selectively bind a particular cell type, for example, a cancer cell or other rare cell. In capture module aspects that include an array of obstacles, the obstacles can include such binding moieties. Additionally, a cell counting module, for example, a Coulter counter, can be fluidically coupled to a separation or enrichment device of the invention. Other modules, for example, a programmable heating unit, can alternatively be fluidically coupled.

The methods of the invention can be employed in connection with any enrichment or analytical device, either on the same device or in different devices. Examples include affinity columns, particle sorters, for example, fluorescent activated cell sorters, capillary electrophoresis, microscopes, spectrophotometers, sample storage devices, and sample preparation devices. Microfluidic devices can be of particular interest in connection with the systems described herein. Exemplary analytical devices include devices useful for size, shape, or deformability based enrichment of particles, including filters, sieves, and enrichment or separation devices, for example, those described in International Publication Nos. 2004/029221 and 2004/113877, Huang et al. Science 304:987-990 (2004), U.S. Publication No. 2004/0144651, U.S. Pat. Nos. 5,837,115 and 6,692,952, and U.S. Application No. 60/703,833, 60/704,067, and Ser. No. 11/227,904; devices useful for affinity capture, for example, those described in International Publication No. 2004/029221 and U.S. application Ser. No. 11/071,679; devices useful for preferential lysis of cells in a sample, for example, those described in International Publication No. 2004/029221, U.S. Pat. No. 5,641,628, and U.S. Application No. 60/668,415; devices useful for arraying cells, for example, those described in International Publication No. 2004/029221, U.S. Pat. No. 6,692,952, and U.S. application Ser. Nos. 10/778,831 and 11/146,581; and devices useful for fluid delivery, for example, those described in U.S. application Ser. Nos. 11/071,270 and 11/227,469. Two or more devices can be combined in series, for example, as described in International Publication No. 2004/029221.

Devices of the disclosure can be adapted for implantation in a subject. For example, such a device can be adapted for placement in or near the circulatory system of a subject in order to be able to process blood samples. Such devices can be part of an implantable system of the invention that can be fluidically coupled to the circulatory system of a subject, for example, through tubing or an arteriovenous shunt. In some cases, systems of the invention that include implantable devices, for example, disposable systems, can remove one or more analytes, components, or materials from the circulatory system. These systems can be adapted for continuous blood flow through the device.

The array can be coupled to a substrate and can reside in a receptacle, which can be coupled to a transparent cover. In some aspects, a sample reservoir can be fluidically coupled to the array and in some aspects a detector can be fluidically coupled to the array. The detector can include, but is not limited to, a microscope, a cell counter, a magnet, a biocavity laser, a mass spectrometer, a PCR device, an RT-PCR device, a matrix, a microarray, or a hyperspectral imaging system. The array can be used to remove an analyte from a cellular sample by processing the sample, preferably continuously. Processing can occur ex vivo or in vivo and can include releasing the analyte from the device by applying a hypertonic solution to the device and detecting the analyte in the effluent from the device.

To reduce non-specific adsorption of cells or particles onto the channel walls, one or more channel walls can be chemically modified to be non-adherent or repulsive. The walls can be coated with a thin film coating (e.g., a monolayer) of commercial non-stick reagents, such as those used to form hydrogels. Additional examples chemical species that can be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers can also be employed to repel oppositely charged species. The type of chemical species used for repulsion and the method of attachment to the channel walls will depend on the nature of the species being repelled and the nature of the walls and the species being attached. Such surface modification techniques are well known in the art. The walls can be functionalized before or after the device is assembled. The channel walls can also be coated in order to capture materials in the sample, e.g., membrane fragments or proteins.

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

Unless otherwise explained, all technical and scientific terms used herein have the same meaning as commonly understood by one of ordinary skill in the art to which this disclosure belongs. The following references contain embodiments of the methods and compositions that can be used herein: The Merck Manual of Diagnosis and Therapy, 18th Edition, published by Merck Research Laboratories, 2006 (ISBN 0-911910-18-2); Benjamin Lewin, Genes IX, published by Jones & Bartlett Publishing, 2007 (ISBN-13: 9780763740634); Kendrew et al. (eds.), The Encyclopedia of Mol. Biology, published by Blackwell Science Ltd., 1994 (ISBN 0-632-02182-9); and Robert A. Meyers (ed.), Mol. Biology and Biotechnology: a Comprehensive Desk Reference, published by VCH Publishers, Inc., 1995 (ISBN 1-56081-569-8).

Standard procedures of the present disclosure are described, e.g., in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl (eds.), Academic Press Inc., San Diego, USA (1987)). Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), and Animal Cell Culture Methods (Methods in Cell Biology, Vol. 57, Jennie P. Mather and David Barnes editors, Academic Press, 1st edition, 1998), which are all incorporated by reference herein in their entireties.

It should be understood that the following examples should not be construed as being limiting to the particular methodology, protocols, and compositions, etc., described herein and, as such, can vary. The following terms used herein are for the purpose of describing particular embodiments only, and are not intended to limit the scope of the embodiments disclosed herein.

Disclosed herein are molecules, materials, compositions, and components that can be used for, can be used in conjunction with, can be used in preparation for, or are products of methods and compositions disclosed herein. It is understood that when combinations, subsets, interactions, groups, etc. of these materials are disclosed and while specific reference of each various individual and collective combinations and permutation of these molecules and compounds cannot be explicitly disclosed, each is specifically contemplated and described herein. For example, if a nucleotide or nucleic acid is disclosed and discussed and a number of modifications that can be made to a number of molecules including the nucleotide or nucleic acid are discussed, each and every combination and permutation of nucleotide or nucleic acid and the modifications that are possible are specifically contemplated unless specifically indicated to the contrary. This concept applies to all aspects of this application including, but not limited to, steps in methods of making and using the disclosed molecules and compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art can recognize, or be able to ascertain using no more than routine experimentation, many equivalents to the specific embodiments of the method and compositions described herein. Such equivalents are intended to be encompassed by the following claims.

It is understood that the disclosed methods and compositions are not limited to the particular methodology, protocols, and reagents described as these can vary. It is also to be understood that the terminology used herein is for the purpose of describing particular embodiments only, and is not intended to limit the scope of the present disclosure which can be limited only by the appended claims.

Unless defined otherwise, all technical and scientific terms used herein have the meanings that would be commonly understood by one of skill in the art in the context of the present specification.

It should be noted that as used herein and in the appended claims, the singular forms “a,” “an,” and “the” include plural reference unless the context clearly dictates otherwise. Thus, for example, reference to “a nucleotide” includes a plurality of such nucleotides; reference to “the nucleotide” is a reference to one or more nucleotides and equivalents thereof known to those skilled in the art, and so forth.

The term “and/or” shall in the present context be understood to indicate that either or both of the items connected by it are involved. While preferred embodiments of the present disclosure have been shown and described herein, it can be obvious to those skilled in the art that such embodiments are provided by way of example only. Numerous variations, changes, and substitutions can now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

### EXAMPLES

### Example 1

### Viable Cell Capture

Prostate tumor cell lines, PC-3, were grown in vitro, and cells were spiked into normal patient blood. The blood was run on a microfluidic device. Instead of fixation, RPMI-1640 cell culture growth medium was added to the chip and the entire chip was then incubated at 37° C. After 1.5 weeks, growing colonies of cells were visible on the surface of the posts (FIG. 32). The ability of the devices to capture viable cells allowed for additional molecular characterization unavailable with platforms that fix the cells prior to capture.

In another experiment, mouse xenograft blood was run across a microfluidic device, washed, the sealing tape removed, and the chip placed in a tissue culture dish in RPMI 1640/10% FBS/penicillin-streptomycin under 5.0% CO2. Cells were imaged by fluorescence and phase-contrast microscopy. At termination of the incubation period, Hoechst 33342 and propidium iodide were added to visualize nuclei and identify dead cells respectively. Ex vivo growth of the isolated tumor cells was evident during the incubation period since the cells spread out and exhibited a flattened morphology. After 12 days in culture, colonies were expanded to the top of the device obstacles. Nearly all cells (>99%) were both GFP positive and nucleated, and <1% were dead. The cultured cells were also stained with Wright-Giemsa to permit clearer examination of the cellular morphology. These methodologies are compatible with capture of viable cells, and these cells can be retained and grown on the microfluidic devices for further analysis steps. Because captured cells are viable, the system can both be used for enumerating CTCs and then adapted to characterize the captured cells for a variety of molecular markers. In addition, the methodologies are amenable to flexibility in processing blood samples at one laboratory site, and shipping microfluidic devices to another site for imaging or other molecular analysis.

Additionally, using an appropriate linker, for example dextran, upon capture of the cells or particles, a reagent will be added, for example dextranase, that removes or cleaves the linker and releases the cells from the obstacles. The released, viable cells will be collected and grown in culture growth medium for further analysis.

### Example 2

### Functionalization of Microfluidic Devices

Microfluidic devices were cleaned and activated with oxygen plasma, incubated with 4% 11-(succinimidyloxy)undecycldimethylethoxysilane (Gelest) in ethanol, washed with ethanol, incubated with 10 μg/mL NeutrAvidin (Pierce) and washed with PBS. This chemistry creates a modified plastic surface amenable to attachment of biomolecules using the avidin-biotin associations. Alternatively, the oxidized chips can be incubated with short dextran chains. Following initial chemical activation, the chips were incubated with 10 μg/ml of a mouse monoclonal anti-EpCAM antibody. Additional other tumor antigen antibodies have been applied at this step as well. Unbound antibodies were washed out prior to processing into storage buffers. The microfluidic device surfaces were stored in a stabilized form by protecting the functionalized microfluidic device batches with a sugar buffer. Many medical devices use a number of sugar-based buffers to stabilize antibodies in an active form. In this application, a sugar buffer consisting of 2 mM L-histidine and 60 mM trehalose (Sigma) was utilized. Microfluidic devices have been immediately assembled following antibody conjugation or have been stored at 4° C., and the use of preservatives is optional. Devices were stored desiccated at 4° C. until use (up to 1 month). Functionalized chips were then fully assembled by first opening two small ports that serve as inlets and outlets of flow. The ports were connected with Tygon® tubing for sample and buffer flow. An adhesive tape was mechanically secured over the post upper surface, creating a chamber for sample flow, washes, and processing.

### Example 3

### Method to Functionalize a Binding Moiety to the Obstacles

The substrate of a microfluidic device was rinsed twice in distilled, deionized water and allowed to air dry. Silane immobilization onto exposed glass was performed by immersing samples for 30 seconds in freshly prepared, 2% v/v solution of 3-[(2 aminoethyl)amino]propyltrimethoxysilane in water followed by further washing in distilled, deionized water. The substrate was then dried in nitrogen gas and baked. Next, the substrate was immersed in 2.5% v/v solution of glutaraldehyde in phosphate buffered saline for 1 hour at ambient temperature. The substrate was then rinsed again, and immersed in a solution of 0.5 mg/mL binding moiety, for example, anti-EpCAM, anti-CD71, anti-CD36, anti-GPA, or anti-CD45, in distilled, deionized water for 15 minutes at ambient temperature to couple the binding agent to the obstacles. The substrate is then rinsed twice in distilled, deionized water, and soaked overnight in 70% ethanol for sterilization.

### Example 4

### Multiparameter Analysis of Biological Samples

Multiparameter analysis of biological samples using configuration with multiple capture entities and multiple characterization modalities included enrichment and characterization of biological blood specimen from cancer patients including:

a) Circulating Tumor cells (with epithelial characteristics)
b) Circulating Tumor cells (with mesenchymal characteristics)

### c) Circulating Cancer Stem Cells

### d) Circulating Mature Endothelial Cells

e) Circulating Progenitor Endothelial cells

Multiparameter analysis of biological sample using configuration with single capture entity and multiple characterization modalities included enrichment and characterization of biological blood specimen from cancer patients (configuration with single capture entity and multiple characterization modalities).

a) Circulating Tumor cells (with apoptotic characteristics—ex. caspase staining)
b) Circulating Tumor cells (with DNA damage—ex. I-12AX staining)
c) Circulating Tumor cells (with multidrug resistance—ex. P glycoprotein staining)
d) Circulating Tumor Cells (with high invasive potential—ex. MMP2 or (MT1)-MMP staining)

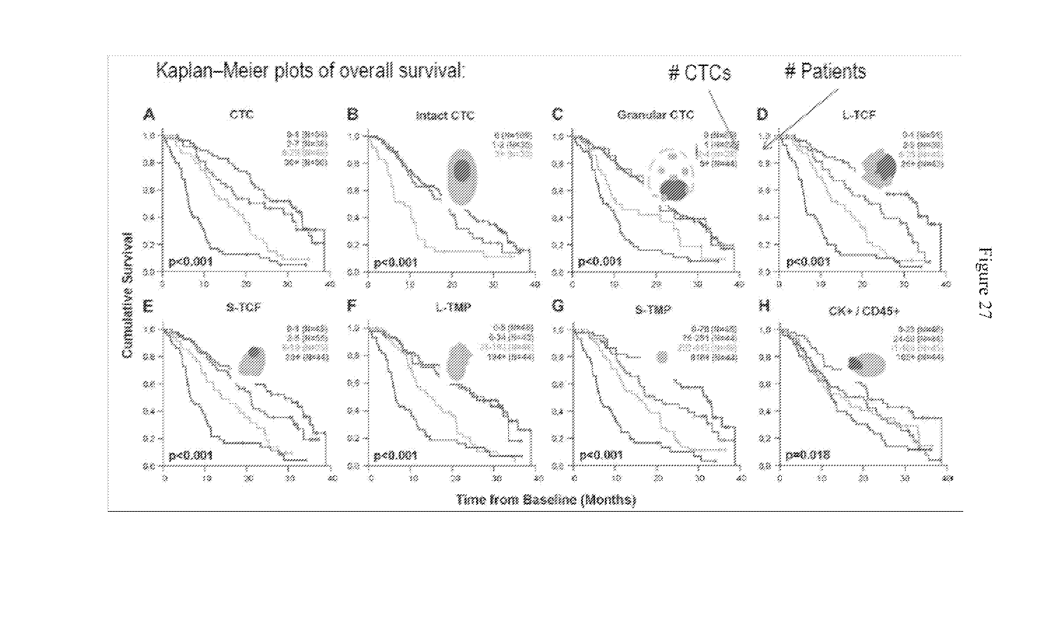

The ratios between biological materials with various characteristics can be used as a diagnostic metric. The microfluidic multichannel enrichment chip allows for the calculation of ratios between different subpopulation of cells, fragments, microparticles, etc. enriched from the same blood sample (or other biological fluid). These ratios can serve as a diagnostic metric for characterization of the disease, choice and success of therapy, prediction of long term survival and disease recurrence as depicted in FIG. 27. As a non-limiting example, patients with increased number of circulating tumor cells with mesenchymal characteristics are likely to have worse prognosis and require augmented therapy.

As one example, patients with increased number of circulating progenitor endothelial cells indicated active neovascularization processes, while increased number of mature circulating endothelial cells (CECs) positively correlated to tumor invasiveness and size, possibly reflecting total tumor vascular volume.

### Example 5

### Identification of Novel Markers to Characterize CTCs and Other Circulating Cells

Characterization of enriched cells from blood utilizes cellular expression of surface markers that have minimal expression in native blood cells. Identification of markers that can successfully identify the targeted cell population, yet are not significantly expressed in blood can be a challenge. A set of markers were identified that allowed for characterization of circulating tumor cells possessing mesenchymal characteristics. These markers were used to identify circulating tumor cells with mesenchymal characteristics in the context of a multichannel microchip but are not limited to it.

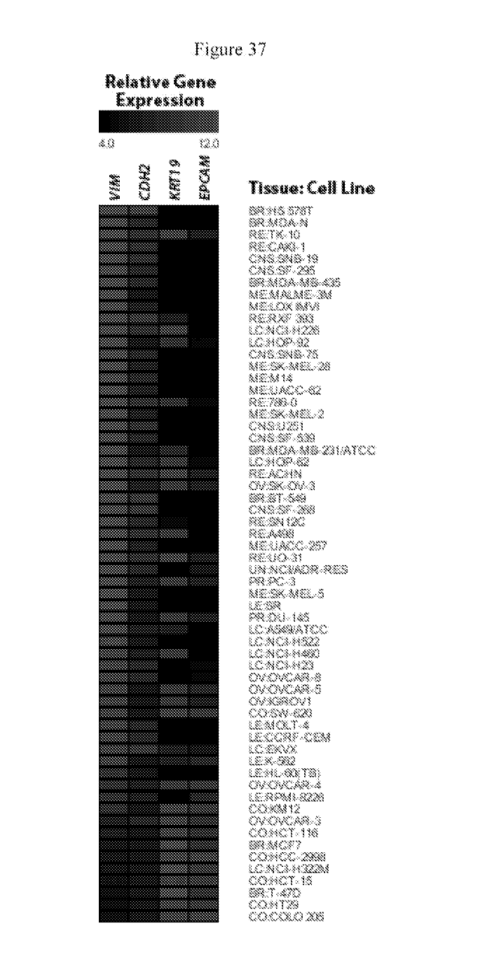

Selection was accomplished by comparative bioinformatical analysis of gene expression data generated as part of NCI-60 cancer cell line characterization program he NCI-60, a panel of 60 diverse human cancer cell lines used by the Developmental Therapeutics Program of the U.S. National Cancer Institute (http://discover.nci.nih.gov/cellminer/). This data was digitally compared to gene expression data obtained for peripheral blood cells from healthy donors (http://www.ncbi.nlm.nih.gov/geo/) as depicted in FIG. 37. Several cell lines characterized as part of the NCI-60 program are known to have mesenchymal characteristics. Cells with mesenchymal characteristics typically have high expression of vimentin (VIM) and N-cadherin (CDH2) and low expression of EpCAM and keratin 19 (KRT19) (FIG. 37). By identifying genes that are highly expressed in cell lines with mesenchymal characteristics (Hs578, MDA-N etc.) but with minimal expression in peripheral blood and cells with epithelial characteristics (Colo205, HT29), we were able to identify markers useful for detection and characterization of circulating tumor cells with mesenchymal characteristics (Table 1).

### Example 6

### Microfluidic Device Manufacturing

The features of a microfluidic device were transferred onto an electroformed mold using standard photolithography followed by electroplating. The mold was used to hot emboss the features into the PMMA at a temperature near its glass transition temperature (105° C.) under pressure (5 to 20 tons) (pressure and temperature were adjusted to account for high-fidelity replication of the deepest feature in the device). The mold was then cooled to enable removal of the PMMA device. A second piece used to seal the device, composed of similar or dissimilar material, was bonded onto the first piece using vacuum-assisted thermal bonding. The vacuum prevents formation of air-gaps in the bonding regions.

### Example 7

In one non-limiting example, standard photolithography was used to create a photoresist pattern of obstacles on a silicon-on-insulator (SOI) wafer. A SOI wafer can be of a 100 μM thick Si(100) layer atop a 1 μm thick SiO2 layer on a 500 μm thick Si(100) wafer. To optimize photoresist adhesion, the SOI wafers can be exposed to high-temperature vapors of hexamethyldisilazane prior to photoresist coating. UV-sensitive photoresist can be spin coated on the wafer, baked for 30 minutes at 90° C., exposed to UV light for 300 seconds through a chrome contact mask, developed for 5 minutes in developer, and post-baked for 30 minutes at 90° C. The process parameters can be altered depending on the nature and thickness of the photoresist. The pattern of the contact chrome mask is transferred to the photoresist and determines the geometry of the obstacles.

Upon the formation of the photoresist pattern that is the same as that of the obstacles, the etching can be initiated. SiO2 can serve as a stopper to the etching process. The etching can also be controlled to stop at a given depth without the use of a stopper layer. The photoresist pattern can be transferred to the 100 Pm thick Si layer in a plasma etcher. Multiplexed deep etching can be utilized to achieve uniform obstacles. For example, the substrate is exposed for 15 seconds to a fluorine-rich plasma flowing SF6, and then the system is switched to a fluorocarbon-rich plasma flowing only C4F8 for 10 seconds, which coats all surfaces with a protective film. In the subsequent etching cycle, the exposure to ion bombardment clears the polymer preferentially from horizontal surfaces and the cycle is repeated multiple times until, for example, the SiO2 layer is reached.

### Example 8

### Process for Producing a Protein Coated Hydrogel Layer on a Solid Support

The present invention provides a process for producing a protein coated hydrogel layer on a solid support comprising the following steps:
1. Treat the solid support (glass or plastic) with oxygen plasma to open reactive binding sites;
2. Prepare a solution comprising mono-functional dextran and PEG and introduce this solution to the surface of the solid support treated in (1);
3. Prepare a solution of bifunctional PEG and introduce this solution to the surface of the solid support treated in (2);
4. Prepare a solution protein (for example antibody, avidin, StreptAvidin, NeutrAvidin, or CaptAvidin) and introduce this solution to the surface of the solid supported treated in (3);
5. Optional: Prepare a solution of biotinylated biomolecules (for example biotin-antibody) and introduce this solution (with or without flow) to the solid support surface treated with avidin, StreptAvidin, NeutrAvidin, or CaptAvidin as prepared in (4).

### Example 9

### Method to Produce Dextran/PEG Based Surface Chemistry with a Covalent Bond NeutrAvidin

Step 1: Oxygen plasma treatment of the surface of the chip (COC) to generate carbonyl groups for the next step.
Step 2: Bind amino dextran to the surface via interaction between its amino group and the surface carbonyl group. The formed N═C bond is then reduced to a single bond for stability.
Step 3: The dextran is then oxidized and the ring opens to form two aldehyde groups, which bind to protein's amino groups.
Step 4: Add NeutrAvidin to the oxidized dextran. The amino group of the NeutrAvindin interacts with the aldehyde groups to form a C═C bond, which is then reduced to maintain NeutrAvidin binding stability.
Step 5: Immobilize one or more biotinylated antibodies through biotin-NeutrAvidin interactions. This can be done in two ways. The biotinylated antibodies can be immobilized in-line right before sample processing by flowing the biotinylated antibody solution into the fully assembled chip. Alternatively, the biotinylated antibodies can be immobilized off-line during the manufacturing process. Antibody preservatives can be added to preserve antibody functionality.

Another way of direct conjugation is to split the antibody into two Fab′ fraction at specific S—S bonds through a controlled reduction. Then the Fab′ is linked to the cross-linker. Due to the location of these S—S bonds, most of the immobilized Fab′ s are in favorable orientation to interact with antigens.

### Example 10

### Method to Produce Dextran/PEG Based Surface Chemistry with Cross-Linked NeutrAvidin

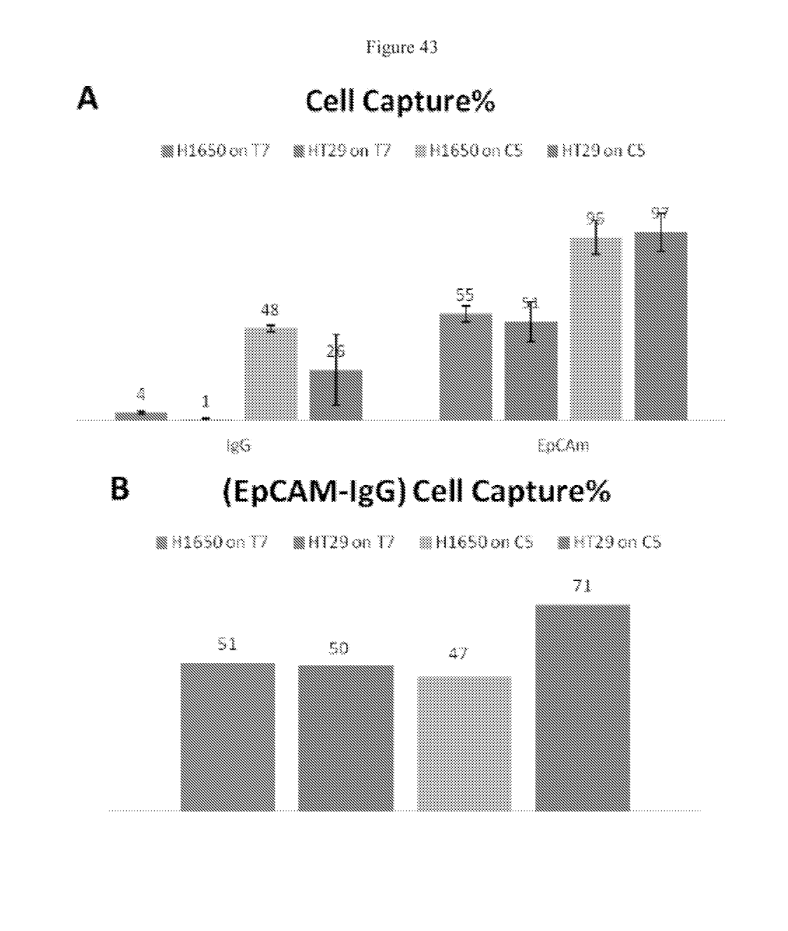

Step 1: Oxygen plasma treatment of the surface of the chip (COC) to generate carbonyl groups for the next step.
Step 2: Bind amino dextran to the surface via interaction between its amino group and the surface carbonyl group. The formed N═C bond is then reduced to a single bond for stability.
Step 3: The dextran remains intact. A hydrophilic cross-linker such as NHS-PEG-Biotin is then used where the amino PEG can have a molecular weight between 2,000 to 20,000 and the bifunctional PEG can have a PEG length of PEG3 or higher at a ratio (dextran:PEG) between 10:1 to 1:10 where the NHS group reacts with the amino groups on dextran (amino dextran).
Step 4: Add NeutrAvidin to the dextran. The biotin group is used to immobilize NeutrAvidin on the surface.
Step 5: Immobilize one or more biotinylated antibodies through biotin-NeutrAvidin interactions. This can be done in two ways. The biotinylated antibodies can be immobilized in-line right before sample processing by flowing the biotinylated antibody solution into the fully assembled chip. Alternatively, the biotinylated antibodies can be immobilized off-line during the manufacturing process. Antibody preservatives can be added to preserve antibody functionality. Both fictionalization approaches significantly reduce non-specific adsorption as after sample processing the chips no longer show significant blue background as in MPS chemistry. The white blood cell count is in the range of 1000-3000/mL of blood instead of tens of thousands, indicating significantly reduced non-specific adsorption. Affinity capture is also improved dramatically. EpCAM antibody coated chips capture more than 40% more cells than IgG antibody coated chips. As depicted in FIG. 43, cell capture performance using two different chip designs with H1650 and HT29 cell lines was evaluated. A) Cell capture percentage on IgG and EpCAM antibody coated chips. (B) The capture percent difference between EpCAM antibody and IgG coated chips. Out of the two approaches including covalent linking and linking using a cross-linker, the cross-linker approach is more efficient as it uses fewer antibodies to achieve the same level of affinity capture. The capture of HT29 cells on T7 is an affinity capture dominated chip which has minimal size-based capture. Only 1-4% of CTCs were captured on IgG coated chips, indicating very few cells were captured by non-specific adsorption. C5 is designed to capture by both size and affinity. IgG chips capture significantly more cells as compared to the T7 chip and the IgG chips capture cells mainly by size. When C5 chips are coated with EpCAM antibody, 50-70% more cells were captured. IgG C5 chips captured 48% of H1650 cells but only 26% HT29 cells. EpCAM antibody chips captured both cells types at an equivalent efficiency. As HT29 cells are smaller in size than H1650 cells, HT29 cells are more difficult to capture by size. T7 results have shown very little capture by non-specific adsorption. These two factors combined cause the 22% drop in size base capture. On the other hand, HT29 cells have higher high EpCAM expression than H1650. The increased EpCAM level and strong affinity based capture compensated for the drop in size-based capture.

### Example 11

### Cross-Linker Approach Compared to Direct-Link Approach

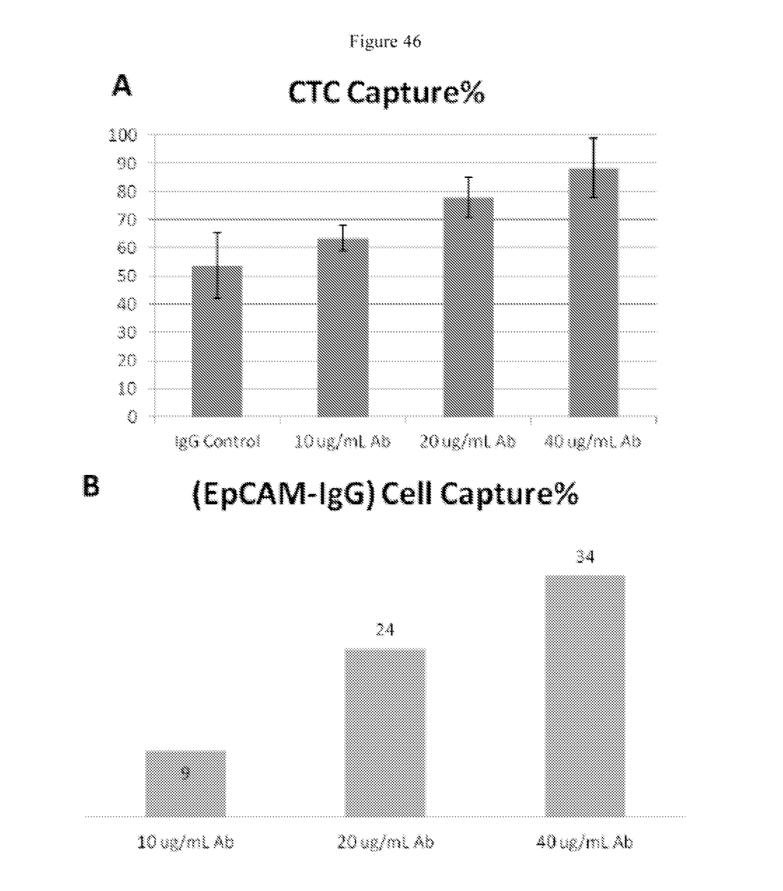

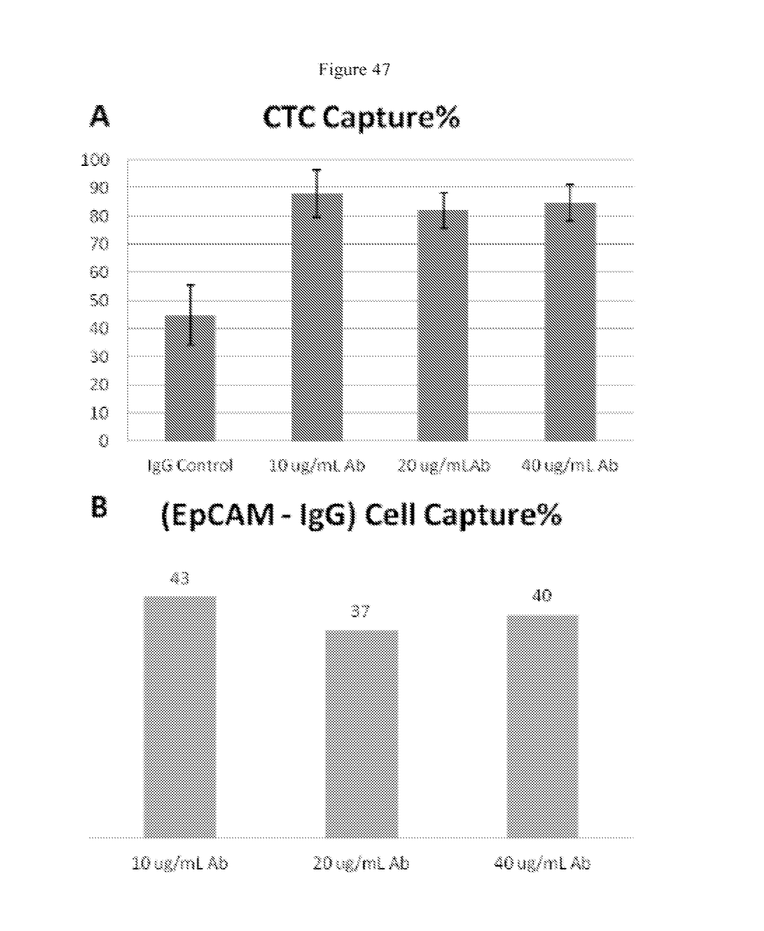

FIG. 46 shows cell capture results when NeutrAvidin is covalently linked to the surface. (A) Cell capture rate of IgG control chips and EpCAM chips at different concentrations. (B) The difference in capture rates of the two chips (C5). FIG. 47 depicts cell capture results when NeutrAvidin is linked to the surface via a hydrophillic cross-linker. (A) Cell capture rate of IgG control chips and EpCAM chips at different concentrations. (B) The difference in capture rates of the two chips (C5). The reason why the cross-linker approach in FIG. 47 is better than the direct-link approach in FIG. 46 is because direct covalent links occur at random amino sites on the protein, which leads to random orientation and is less efficient at immobilization due to less freedom of the protein due to steric hindrances. The other downside to the direct link approach is the possibility of multiple links per NeutrAvidin, which may lead to increased non-specific adsorption. As depicted in FIG. 48, the direct-linked IgG chip captures a significant amount of CTCs mainly by non-specific adsorption. The hydrophilic cross-linker also improves affinity capture as depicted in the EpCAM chips in FIG. 48. In the direct link approach, the inlet side of EpCAM chip captures more CTCs than the IgG chip, but only by a small percentage. Although the capture rate is significantly higher, the capture happens across and chip and concentrates towards the outlet side. This indicates in the direct-link approach, for the affinity capture to work efficiently, the size factor still plays an important role. This is not the case in the cross-linker approach, where a major shift in capture mechanism is observed. Comparing to the IgG chip, the EpCAM chip of cross-linker approach captures a significant amount of cells at the inlet side where size capture is minimal. This fact that the IgG chip of cross-linker approach captures few cells in the inlet side makes the phenomenon more evident. Comparing the two EpCAM chips, the shift is also apparent. The direct-link chips capture cells mainly on the size dominated side. The cross-linker chip captures cells mainly on the affinity dominated side. This demonstrates the superior performance of the cross-linker approach.

### Example 12

### Amino-Dextran Functionalization

Native dextran has no amino group. An amine functionalized dextran is used as the amino groups will react with surface carbonyl groups on plasma oxidized COC. Generally a thick layer of dextran coating (higher MW dextran) is more desirable as it fully covers the plastic surface, blocking interference of the plastic to the analytes. Commercially available amino dextran has more amino groups per dextran as dextran molecular weight (MW) increases. For example, 10 K MW amino dextran has 2-5 amino groups. 40K amino dextran has about 10 amino groups and 70K amino dextran has close to 20 amino groups. Amino groups facilitate the immobilization of amino dextran on the surface, but too many amino groups per dextran molecule may be detrimental as 70K dextran captures ˜20% more white blood cells (WBCs) than 40K dextran without improving in CTC cell capture. Although pinpointing the exact mechanism causing increased WBC adsorption is difficult, one may speculate that as one dextran molecule binds to more surface sites, the conformation of the dextran coating may become less favorable. 10K amino dextran adsorbs 30% fewer WBCs than 40K amino dextran. But higher MW dextran has its advantages in terms of improving antibody performance. In addition to blocking non-specific binding, higher MW dextran also pushes the antibody farther away from the surface and gives the antibody more flexibility to rotate in a 3D space and better interact with cell surface antigens. These two factors increase efficient capture of rare cells or other analytes in blood samples. Thus, 40K dextran can have advantages over 10K dextran.

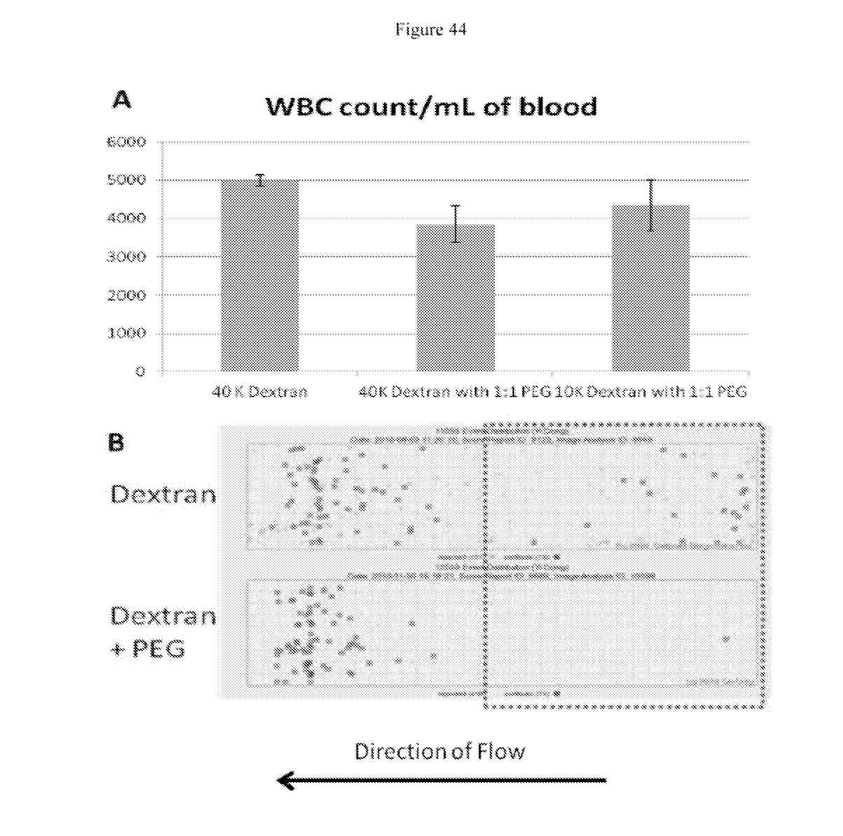

One way of solving this problem is to add a reagent that competes with dextran's amino group. PEG-amine has been utilized because it's long hydrophilic chain is favorable for blocking non specific adsorption while the amine end will compete with dextran's amino group. FIG. 44 shows the effect of adding PEG on reducing WBC counts, as the 40K-MW dextran mixed with an equal molar amount of PEG-amine caught about 25% fewer WBCs. Another benefit of the added PEG is reduced non-specific capture of CTCs. As samples with CTCs flow through the C5 chip from right to left, the CTCs first enter the affinity capture zone (dotted area) and then the size capture zone. On an IgG-dextran coated chip a small number of cells are captured in the affinity capture zone due to non-specific adsorption. When PEG is added to the dextran layer, this non-specific capture is reduced along with WBCs as depicted in FIG. 44.

PEG hydrogel has been used for immunoassay type applications on microfluidic chips. Most PEG polymers created through a commercial process exist as a distribution of chain lengths. The MW of many commercial PEG and PEG derivatives is an average MW. PEG used in surface coating usually has at least two functional groups. One functional group binds to the assay substrate and the other is used to immobilize capture modules of the analyte. Thus there is a difference between the conventional use of PEG as a substrate and the use of PEG in the present disclosure. In the present disclosure PEG amine's amino head groups compete with that of amino-dextran and reduce the number of bonds between each dextran molecule and the plastic surface. This increases the effective thickness of the dextran layer and improves dextran chain flexibility, both of which are important for optimal antibody performance. When bifunctional PEG is used to replace the dextran/PEG layer, the resulting chips capture 88.6% of CTCs on C5.4 chips while the dextran/PEG chips capture 96.3% of CTCs.

### Example 13

### Effect of Added BSA on Antibody Immobilization

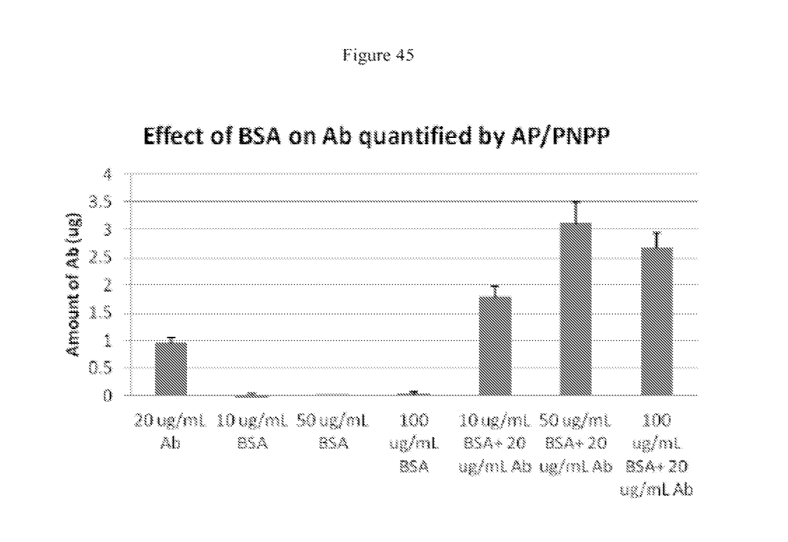

FIG. 45 depicts the effect of added BSA on the amount of antibodies immobilized on the chip surface as quantified by alkaline phosphatase/PNPP assay. Different amounts of BSA were mixed with EpCAM antibodies to reach BSA concentration of 10, 50, and 100 μg/mL while the antibodies concentration is maintained at 20 μg/mL. Then the surface bond antibodies are quantified and compared with pure antibody and pure BSA solutions. As shown in FIG. 45, BSA does not cause any significant amount of false positive signal through the range, but it does increase the amount of antibodies as quantified by the assay which quantifies the amount of active antibodies. A 500 μL antibody solution at 20 μg/mL was used. This means that about 10% (1/0.5*20) of the antibodies are effectively immobilized. The addition of BSA significantly improves this process. 50 μg/mL BSA with 20 μg/mL antibody yielded three times as many active antibodies. EpCAM antibody coated chips captured 19% more CTCs than IgG coated control chips, which is a significant improvement upon the previous 5% difference.

### Example 14

### MPS Functionalization Chemistry

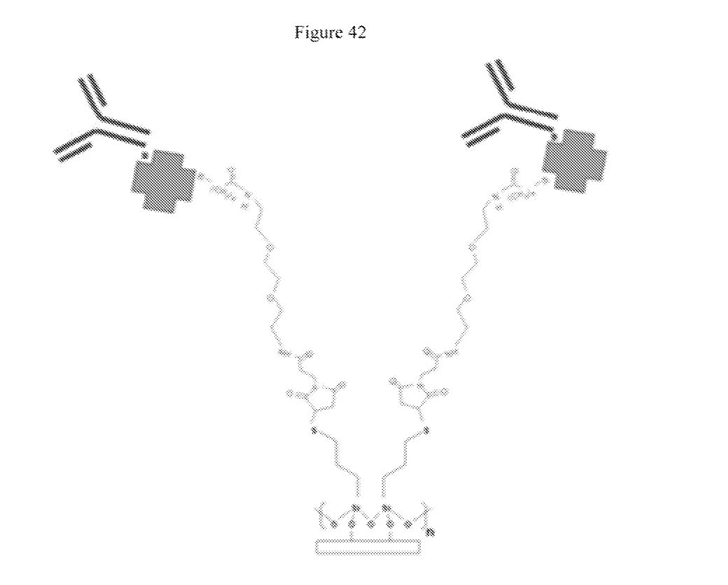

A schematic of MPS chemistry is shown in FIG. 42. Briefly, the process includes the following steps:
Step 1: The COC surface is oxidized with oxygen plasma
Step 2: (3-Mercaptopropyl)trimethoxysilane) (MPS) is added to the surface of the chip and binds to the plasma treated surface and provides thiol functional groups
Step 3: Maleimide-PEG2-biotin is added and binds to the thiol groups via interaction between maleimide and thiol, and presents biotin.
Step 4: NeutrAvidin is added and binds to the biotin moieties
Step 5: Biotinylated EpCAM antibodies are added and bind to the NeutrAvidin moieties
Step 6: Antibody preservative is added.

Unlike glass The MPS chemistry is a proper process on glass slides. Unlike glass or silicon/silica, which is hydrophilic, the COC plastic is hydrophobic and can be sensitive to organic reagents and solvents used in MPS chemistry. A highly hydrophobic service works well in blocking non-specific binding and increasing antibody activity by providing a conducive-surface environment. The MPS process can improve surface hydrophilicity, although non-specific binding still occurs.

### Example 15

### Validation of C5.2

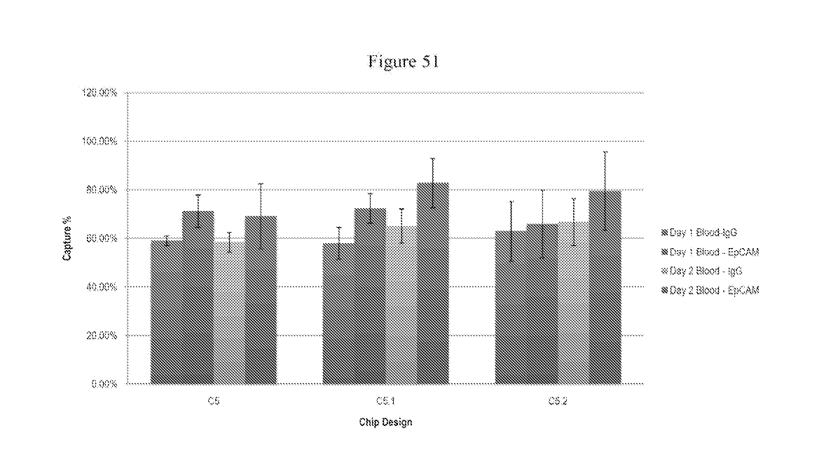

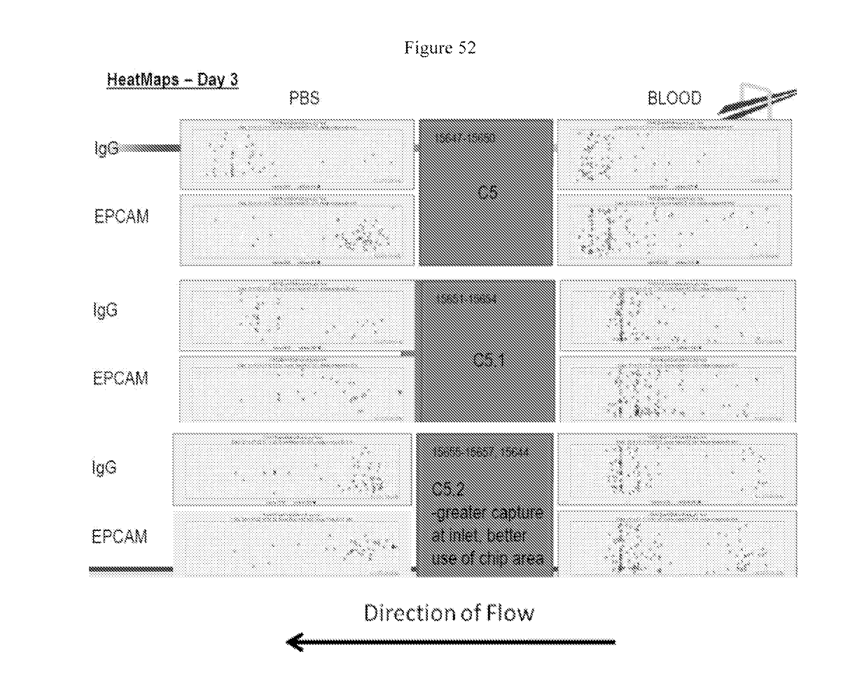

Blood samples with CTCs were processed using the previous C5 design or the new C5.1 and C5.2 designs on two separate days. The chips tested were coated with either IgG or EpCAM antibodies and the sample was processed at a flow rate of 25 μL/min. As shown in FIG. 51, the C5.1 and C5.2 designs show equivalent or greater capture efficiency compared to original C5 design at 25 μL/min. As shown in FIG. 52, the C5.2 design demonstrated greater capture of cells in either PBS or blood samples near the inlet, and thus better use of the entire chip area than the C5 and C5.1 designs.

### Example 16

### Optimization of Capture Conditions

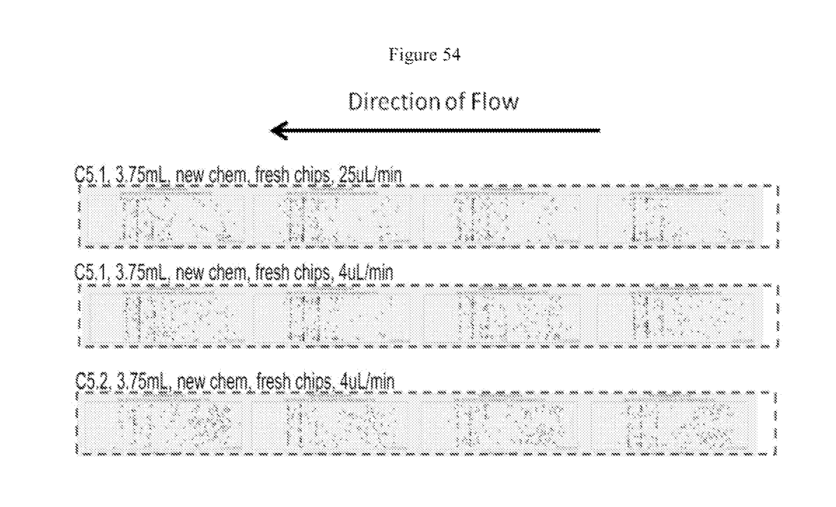

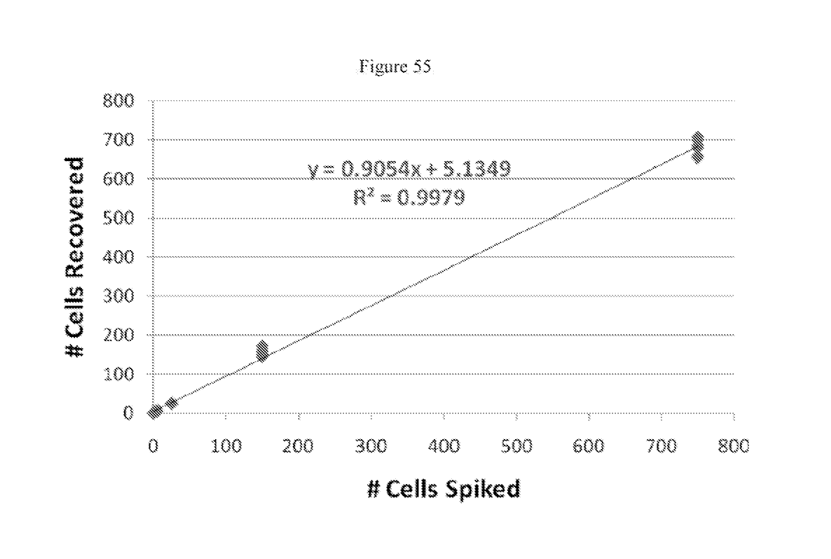

In order to improve overall capture efficiency, antibody capture, and utilization of chip area to distinguish between affinity and size capture a series of conditions were explored: capture flow rates of 25 μL/min, 7.6 μL/min and 4 μL/min, old vs. new surface chemistries, and running whole blood vs. removing serum from the sample. The C5.1 and C5.2 designs previously demonstrated equivalence to C5 at previous processing conditions (25 μL/min), so C5.1 was chosen as platform for running new conditions and compared to C5.2. As seen in FIG. 53, the C5.2 design captured a higher percentage of cells spiked into the samples for all conditions tested. Furthermore, as shown in FIG. 54, the C5.2 design demonstrated improved utilization of chip area at slower flow rates (better capture at inlet, better distribution of cells across chip), and improved inlet capture (indicative of capture by affinity) compared to the C5.1 design. Additionally, the C5.2 demonstrated robust reproducibility (data not shown). Furthermore, the C5.2 chip design demonstrated linear capture of cells spiked into the sample ranging from 0-750 spiked cells as shown in FIG. 55.

### Example 17

### C5.3 and C5.4 Validation Experiments

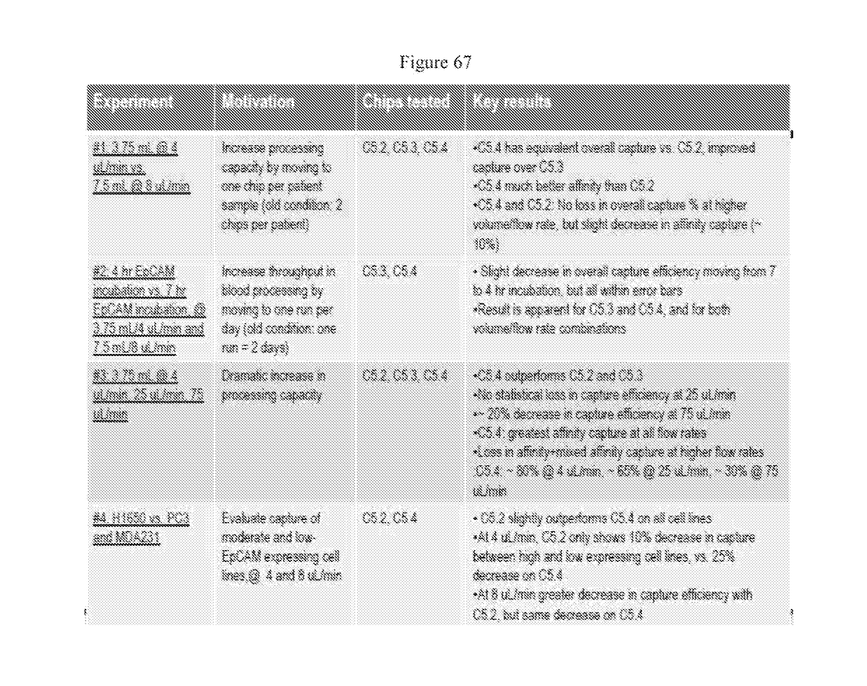

Multiple experiments were carried out to validate the C5.3 and C5.4 designs and to compare to previous designs. These experiments tested various parameters comprising sample volumes, flow rates, binding moiety incubation and surface conjugation time, and capture efficiency of various cells lines with high, low, and moderate EpCAM expression. A summary of the experiments, motivation behind performing the experiments, chips tested, and results can be seen in FIG. 67.

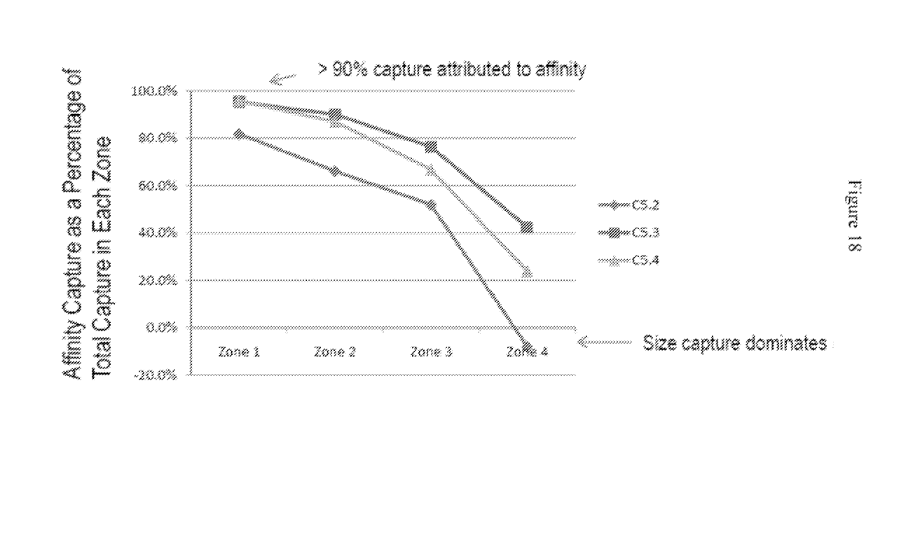

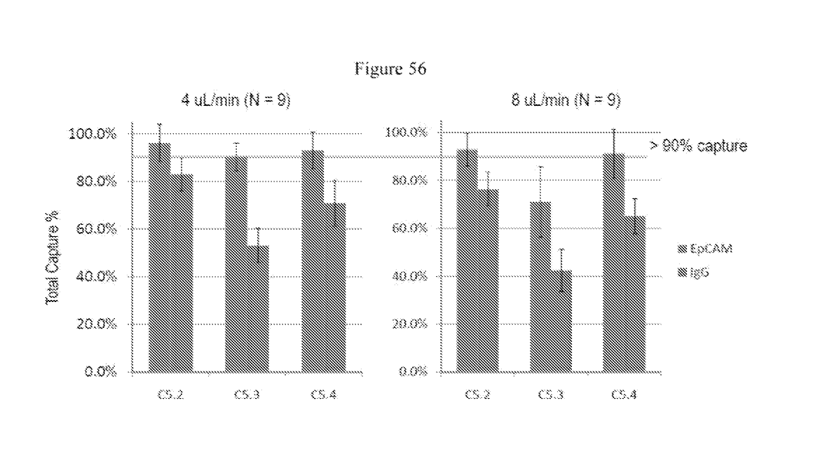

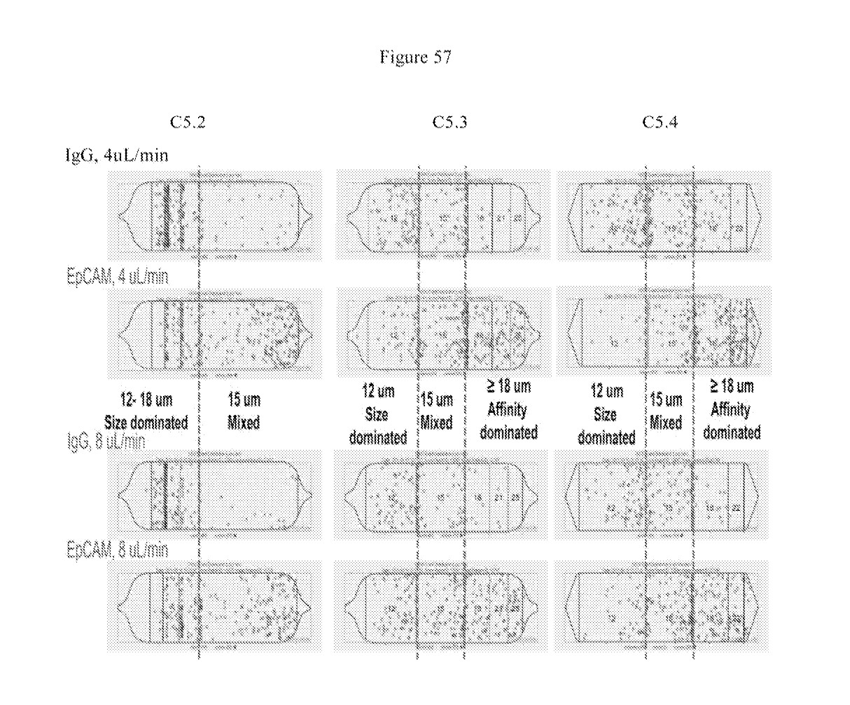

In one experiment, samples spiked with a known number of CTCs were processed on the C5.2, C5.3, and C5.4 designed chips functionalized with either IgG or EpCAM antibodies at a flow rate of either 4 μL/min, or 8 μL/min. As shown in FIG. 56, all EpCAM functionalized designs had 90%+ capture rate at 4 μL/min. The data indicates that C5.2 and C5.4 can process larger volumes at 8 μL/min as they maintained 90%+ capture rates. As depicted in the capture plots in FIG. 57, all chip designs showed a shift in the spatial localization of cells between IgG and EpCAM capture at 4 μL/min and 8 μL/min which can be a positive indication of affinity capture even at higher volume/flow combination. To quantify affinity based capture, size based capture, or a combination thereof, the capture rate of each zone within the array of the chip types can be determined as shown in FIG. 16 and FIG. 18. The total capture is equal to the sum of the cells captured by affinity plus the cells captured by size, which are determined by the region in which the cells are captured. Affinity capture can be calculated as a proportion of total capture or as a proportion of total capture in each zone. The affinity capture was quantified from EpCAM functionalized chips and the data can be seen in FIG. 16 (top). The C5.3 and C5.4 chips using EpCAM showed improved affinity capture vs. C5.2 chips, and a significant portion of capture (>70% on C5.3, >80% on C5.4) can be attributed to affinity capture while the remaining capture is size dominated, and even at a higher volume/flow rate, both C5.3 and C5.4 were dominated by affinity capture. When chips using IgG were used (FIG. 16, bottom) size capture dominated and affinity capture was diminished on all three chip types. Comparing EpCAM and IgG capture rates in each zone, affinity capture can be calculated as a proportion of total capture in each zone as depicted in FIG. 18. The C5.3 and C5.4 chip designs showed much stronger affinity capture across all zones compared with the C5.2 chip design as over 80% capture can be attributed to affinity in zones 1 and 2 of C5.3 and C5.4. In summary the C5.4 chip design has equivalent overall capture vs. the C5.2 chip design and improved capture over the C5.3 chip design, the C5.4 chip design has much better affinity than the C5.2 chip design, and the C5.4 and C5.2 chip designs exhibited no loss in overall capture % at higher a volume/flow rate, but a slight decrease in affinity capture (˜10%).

### Example 18

### C5.3 and C5.4 Validation Experiments

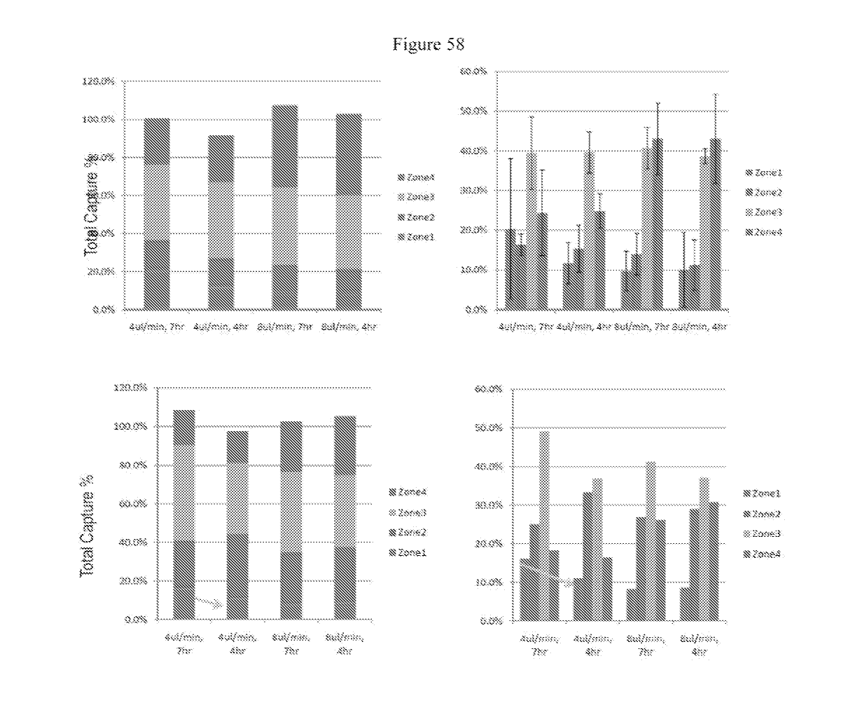

In another experiment the flow rate/sample volume and EpCAM incubation time was tested to determine if less than 7 hours of incubation time could be utilized. The motivation behind the experiments was to increase throughput in blood processing by moving to shorter incubation times and under 24 hrs per run. These experiments tested sample volumes and flow rates of 3.75 mL at 4 μL/min and 7.5 mL at 8 μL/min with EpCAM functionalized C5.3 and C5.4 chips using 3 different blood samples per day for 3 days. As shown in FIG. 22, over three days there was no significant difference in total average capture for 4 hours versus 7 hours of EpCAM antibody incubation time for the conditions tested. The spatial localization of cells captured on the C5.4 chip design can be seen in FIG. 23. For C5.3 and C5.4 chip designs, 7 hr and 4 hr incubations seem to be different as shown in FIG. 58 (C5.3—top, C5.4—bottom). The effect showed up in the most affinity dominated capture region (zone 1) and lowest flowrate (4 μl/min) as a drop in capture rate. The difference diminished under 8 μL/min as affinity capture rates for both incubation times drop. The overall results from this experiment showed a slight decrease in overall capture efficiency moving from 7 hr to 4 hr incubation, but all within error, and these results are apparent for C5.3 and C5.4, and for both volume/flow rate combinations.

### Example 19

### C5.3 and C5.4 Validation Experiments

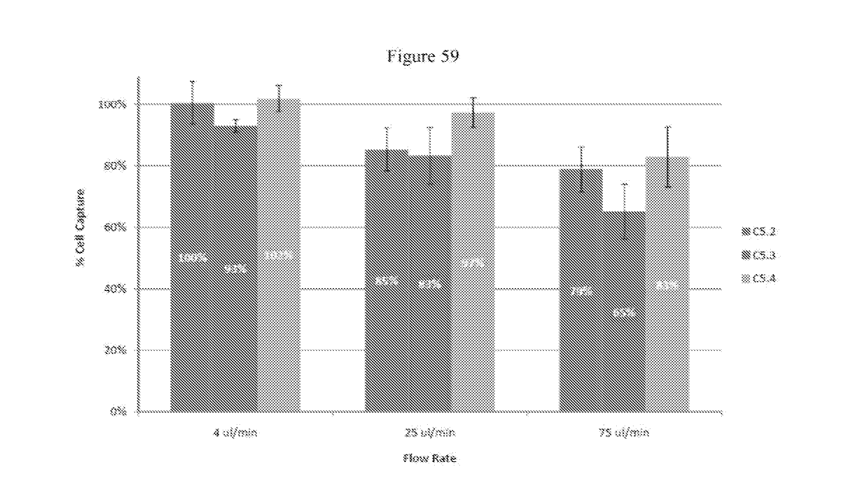

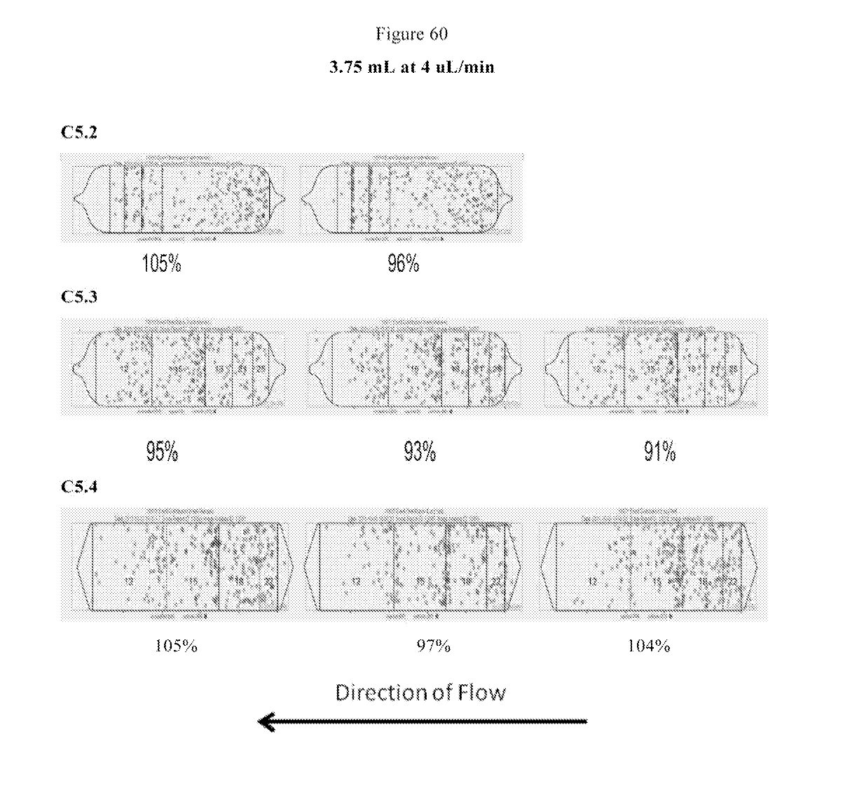

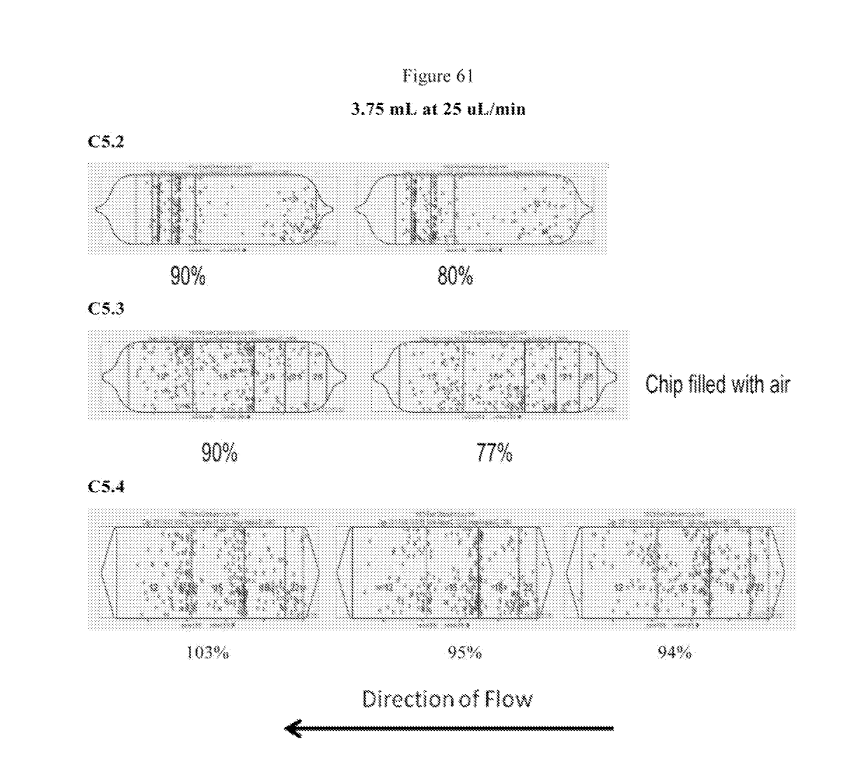

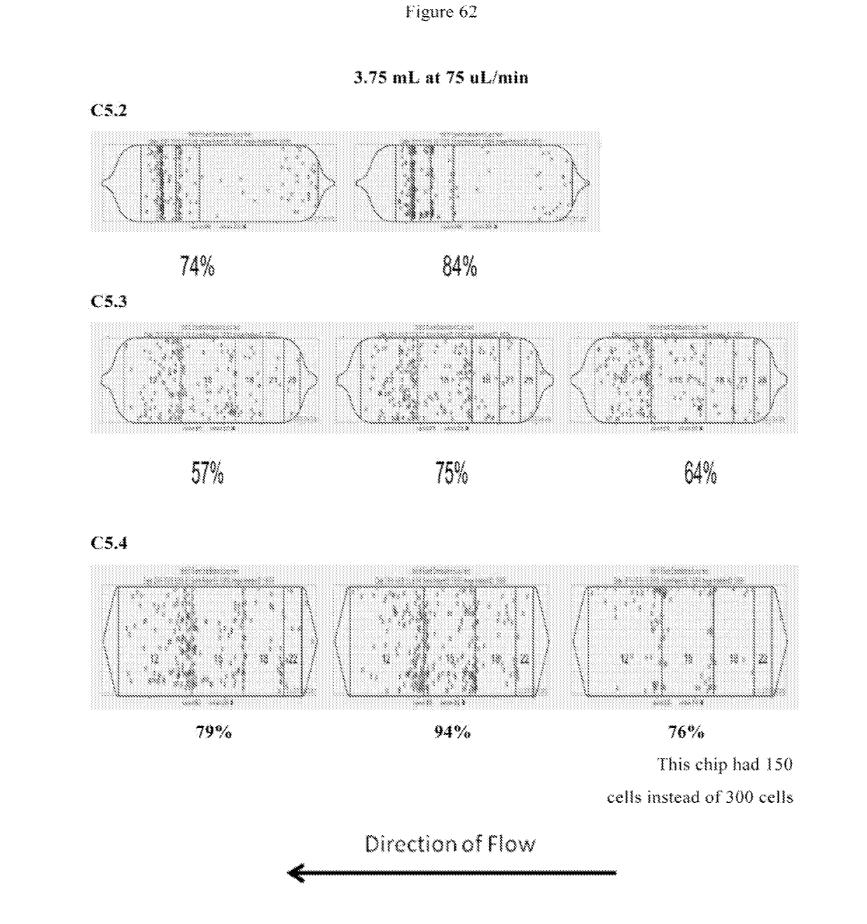

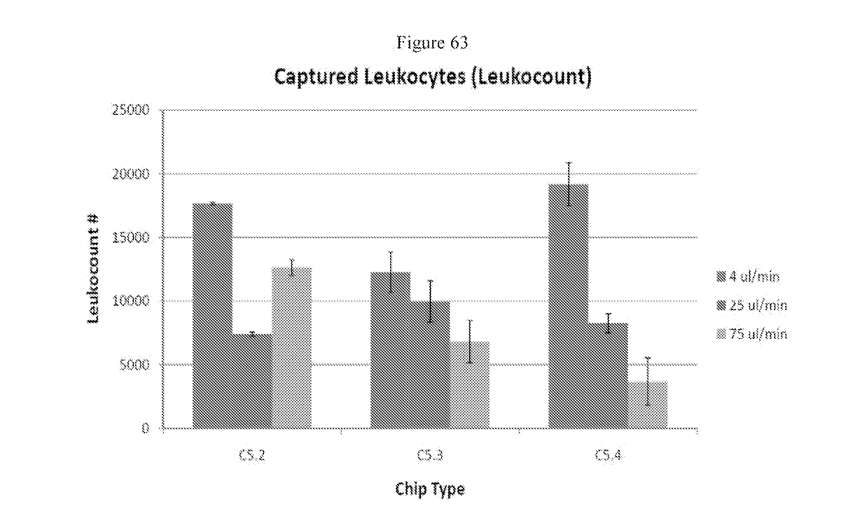

In a third experiment the performance of the C5.2, C5.3, and C5.4 chip designs functionalized with EpCAM were evaluated at higher flow rates including 3.75 mL of blood sample at 4 μL/min, 25 μL/min, and 75 μL/min flow rates under the same antibody incubation times using three different blood samples. As shown in FIG. 59, the capture percentage of 300 total spiked H1650 cells in 3.75 mL blood is significantly reduced on designs C5.2 and C5.3, but not C5.4, at 25 μl/min. The spatial localization of cells captured under these conditions can be seen in FIG. 60, FIG. 61, and FIG. 62. The affinity, size, and mixed (affinity and size) capture percentage was quantified from these EpCAM functionalized chips under these conditions and the data can be seen in FIG. 20. Overall, the affinity component decreased as the flow rate increased but the size component compensated for this loss of affinity capture. The affinity plus mixed capture components was approximately twice as high on the C5.4 chip design than the C5.2 chip design at 25 μl/min. The number of captured leukocytes from the blood (non-specific capture) was also evaluated under the above conditions on the chips and it was found that the leukocyte background was significantly reduced at the higher flow rates using 3.75 mL samples (FIG. 63). In summary, the C5.4 chips outperformed the C5.2 and C5.3 chips and exhibited the greatest affinity capture at all flow rates tested. No statistical loss in capture efficiency was observed at a flow rate of 25 μl/min and about a 20% decrease in capture efficiency was observed at a flow rate of 75 μl/min.

### Example 20

### C5.3 and C5.4 Validation Experiments

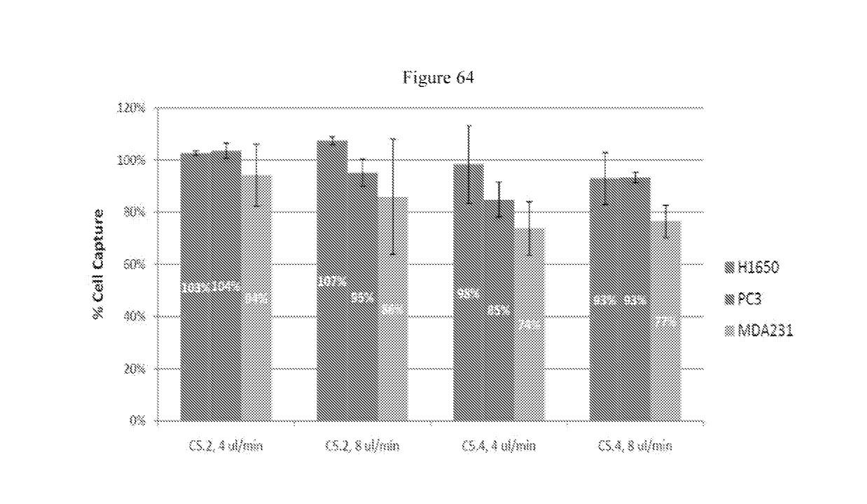

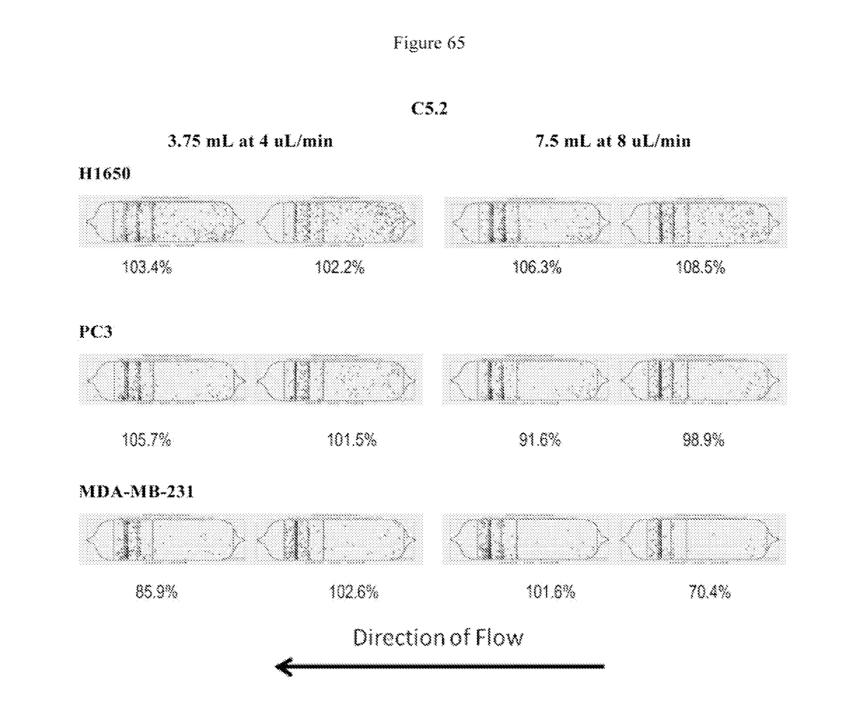

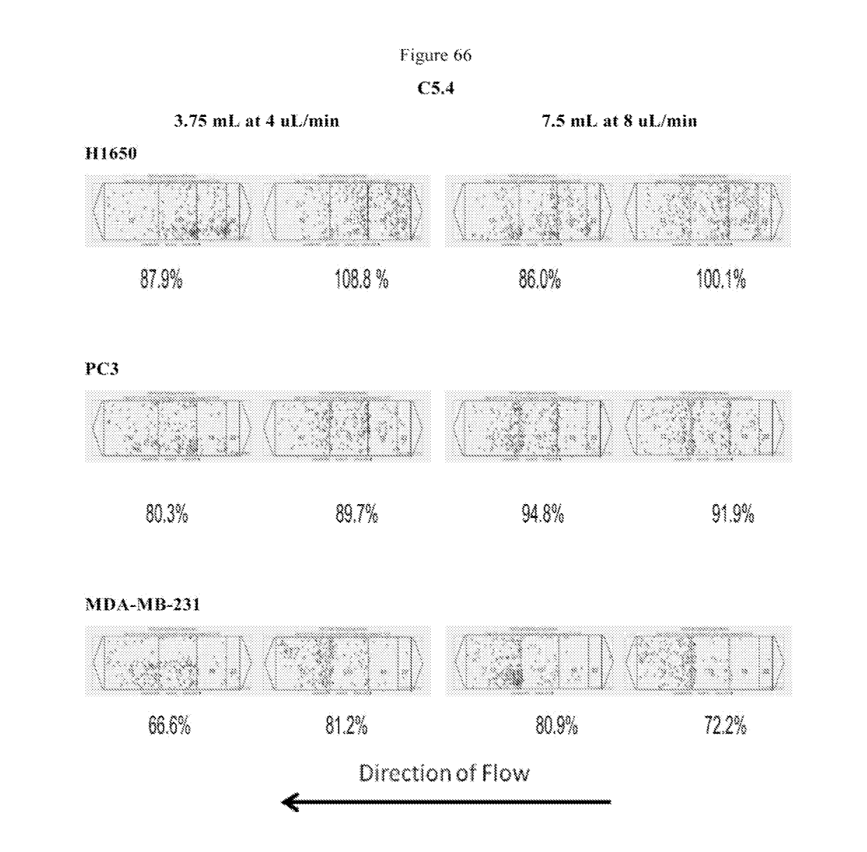

In a fourth experiment the performance of 3 cell lines on the C5.2 and C5.4 chip designs functionalized with EpCAM were evaluated using 4 different blood samples with a volume of 3.75 mL at a flow rate of either 4 μl/min or 8 μl/min using the same number of spiked cells under the same processing conditions. In these experiments, H1650 cells, known to express high EpCAM levels, PC3 cells, known to express moderate to low EpCAM levels, and MDA-MB-231 cells, known to express very low EpCAM levels were spiked into the blood samples. The percent cell capture was quantified and the results can be seen in FIG. 64. Overall, the C5.4 chip exhibited a slightly lower percent cell capture than C5.2 chips for H1650 cells (consistent with previous results) and PC3 cells. A more significant difference in performance on the two chip types for MDA-231 cells was observed where presumably size capture plays more of a role. However, for the lower EpCAM level expressing cells (PC3 and MDA-231), the change in flow rate from 4 μl/min to 8 μl/min caused a 8-9% decrease in capture on C5.2 chips, and no decrease in capture on C5.4 chips, suggesting the C5.4 chip design is much more capable of handling higher flow rates. The spatial localization of cells captured under these conditions can be seen in FIG. 24 (3.75 mL whole, normal blood, processed at 4 μL/min), FIG. 25 (7.5 mL whole, normal blood, processed at 8 μL/min), FIG. 65 and FIG. 66. For the 3.75 mL blood samples processed at 4 μL/min, there was a notable shift in capture toward the outlet that correlated with decreased EpCAM expression but there was no significant difference in overall capture. For the 7.5 mL blood samples processed at 8 μL/min, there was a notable shift in capture toward the outlet that correlated with decreased EpCAM expression and a reduced capture of MDA-MB-231 cells compared to the capture using the C5.2 chip design with 3.75 mL blood samples processed at 4 μL/min. In summary, the C5.2 chip design performed better than the C5.4 chip design for all cell lines tested. At a flow rate of 4 μL/min, the C5.2 chip design shows a 10% decrease in capture between high and low expressing cell lines, compared to a 25% decrease on C5.4 chip designs. At a flow rate of 8 μL/min, a greater decrease in capture efficiency was observed with C5.2 chips and the same decrease was observed on C5.4 chips.

1.-53. (canceled)

54. A microfluidic device comprising an array of obstacles coated with antibodies wherein a surface of the device is functionalized with dextran or dextran derivatives.

55. A microfluidic device comprising an array of obstacles coated with antibodies wherein a surface of the device has a contact angle of less than 15° over at least 10 hours.

56.-71. (canceled)

72. A method for capture and release of cells or cell fragments of interest, the method comprising: (a) flowing a sample comprising cells or cell fragments of interest on a surface coated with carbohydrate and binding moiety that selectively binds a cell surface marker selectively present on the cells or cell fragments of interest, and (b) using an enzyme that selectively cleaves the carbohydrate or a biotin derivative wherein the biotin derivative competitively releases biotin conjugates, or both, to thereby release the cells or cell fragments of interest from the surface.

73. The method according to claim 72, wherein a biotin derivative that competitively releases biotin conjugates comprises desthiobiotin.

74. The method according to claim 72, wherein an enzyme that selectively cleaves the carbohydrate comprises dextranase, a glycosyltransferase, a glycoside hydrolase, a transglycosidase, a phosphorylase, or a lyase.

75.-78. (canceled)

79. The device of claim 54, wherein the device is capable of capturing at least 60% of circulating tumor cells in a sample when a concentration of the antibodies in a solution that coat the array of obstacles is between about 10 μg/mL to about 100 μg/mL.

80. The device of claim 54, wherein a concentration of the dextran or the dextran derivatives in a solution used to functionalize the surface of the device is from 0.01% to 5% (w/w) or from 0.05% to 2% (w/w).

81. The device of claim 54, wherein the antibodies coated on the array of obstacles are capable of selectively capturing cells, cell fragments, particles, or any combination thereof.

82. The device of claim 81, wherein the cells comprise epithelial cells, non-epithelial cells, non-epithelial tumor cells, cells undergoing epithelial to mesenchymal transition, cancer stem cells, mesenchymal cells, or any combination thereof.

83. The device of claim 81, wherein the cell fragments comprise proteins or nucleic acids.

84. The device of claim 55, wherein the surface of the device is functionalized with a carbohydrate.

85. The device of claim 84, wherein the carbohydrate comprises dextran, chitin, chitosan, alginate, cellulose, methylcellulose, starch, heparin, agarose, concanavalin A, callose, laminarin, chrysolaminarin, xylan, arabinoxylan, mannan, fucoidan, galactomannan, or any combination thereof.

86. The device of claim 84, wherein a concentration of the carbohydrate in a solution used to functionalize the surface of the device is from 0.01% to 5% (w/w), 0.01% to 4% (w/w), 0.01% to 3.75% (w/w), 0.01% to 3.5% (w/w), 0.01% to 3.25% (w/w), 0.01% to 3% (w/w), 0.01% to 2.75% (w/w), 0.01% to 2.5% (w/w), 0.01% to 2.25% (w/w), 0.01% to 2% (w/w), 0.01% to 1.75% (w/w), 0.01% to 1.5% (w/w), 0.01% to 1.25% (w/w), 0.01% to 1% (w/w), 0.01% to 0.75% (w/w), 0.01% to 0.5% (w/w), 0.01% to 0.25% (w/w), 0.05% to 2% (w/w), 0.05% to 1.9% (w/w), 0.05% to 1.8% (w/w), 0.05% to 1.7% (w/w), 0.05% to 1.6% (w/w), 0.05% to 1.5% (w/w), 0.05% to 1.4% (w/w), 0.05% to 1.3% (w/w), 0.05% to 1.2% (w/w), 0.05% to 1.1% (w/w), 0.05% to 1% (w/w), 0.05% to 0.9% (w/w), 0.05% to 0.8% (w/w), 0.05% to 0.7% (w/w), 0.05% to 0.6% (w/w), 0.05% to 0.5% (w/w), 0.05% to 0.4% (w/w), 0.05% to 0.3% (w/w), 0.05% to 0.2% (w/w), or 0.05% to 0.1% (w/w).

87. The method according to claim 72, wherein the binding moiety is covalently bonded to the carbohydrate.

88. The method according to claim 72, wherein the binding moiety is bonded to the carbohydrate via a linker.

89. The method according to claim 72, wherein the binding moiety is an affinity tagged ligand.

90. The method according to claim 72, wherein the binding moiety is selected from the group consisting of: avidin, NeutrAvidin, StreptAvidin, or CaptAvidin.

91. The method according to claim 72, wherein the binding moiety enables capture of epithelial cells, non-epithelial cells, non-epithelial tumor cells, cells undergoing epithelial to mesenchymal transition, cancer stem cells, mesenchymal cells, cellular fragments, proteins, nucleic acids particles, or any combination thereof.

92. The method according to claim 72, wherein the carbohydrate is dextran, chitin, chitosan, alginate, cellulose, methylcellulose, starch, heparin, agarose, concanavalin A, callose, laminarin, chrysolaminarin, xylan, arabinoxylan, mannan, fucoidan, galactomannan, or any combination thereof.

93. The method according to claim 92, wherein a concentration of the carbohydrate in solution that is dextran used to coat the surface is from 0.01% to 5% (w/w) or from 0.05% to 2% (w/w).
